(19) United States
(12) Patent Application Publication
(10) Pub. No.: US 2026/0216156 A1
(43) Pub. Date: Jul. 30, 2026

(54) METHODS AND COMPOSITIONS FOR THE TREATMENT OF HEARING DISORDERS

(71) Applicant: Gateway Biotechnology, Inc., Durham, NC (US)

(72) Inventors: Jianxin BAO, St. Louis, MO (US); Stephen H. WU, Chesterfield, MO (US); David STADERMANN, Princeton, NJ (US)

(21) Appl. No.: 19/145,083

(22) PCT Filed: Jan. 5, 2024
(86) PCT No.: PCT/US2024/010577

§ 371 Date: Jul. 1, 2025

Related U.S. Application Data
(60) Provisional application No. 63/478,883, filed on Jan. 6, 2023.

(51) Int. Cl. A61K 31/4422; A61K 9/00; A61K 31/55; A61K 47/10; A61K 47/12; A61K 47/18; A61P 27/16

(52) U.S. Cl. CPC A61K 31/4422; A61K 9/0043; A61K 31/55; A61K 47/10; A61K 47/12; A61K 47/186; A61P 27/16

(57) ABSTRACT

Provided are methods and compositions for the treatment of neural disorders, such as hearing disorders. The methods can comprise a method for treating tinnitus or Meniere's disease or a symptom of tinnitus or Meniere's disease in a subject in need thereof by intranasally administering a therapeutically effective amount of NMDP or a salt thereof. Compositions can comprise an intranasal delivery formulation of a therapeutically effective amount of NMDP or a salt thereof for the treatment of neural disorders.

### CROSS REFERENCE

This application claims the benefit of U.S. Provisional Application No. 63/478,883, filed on Jan. 6, 2023, which is incorporated herein by reference in its entirety.

### BACKGROUND

Hearing disorders are a growing health problem, with varied and complex etiology. While some forms of hearing disorders are clearly genetic in origin, others are either wholly or at least partially environmental in nature.

One such common hearing disorder is tinnitus; a ringing of the ears that can lead to fatigue, stress, trouble sleeping, trouble concentrating, memory problems, depression, anxiety, irritability, and headaches, among other problems, making it a debilitating condition. Tinnitus affects about 20% of the population, and is especially common in older adults, or in people who are routinely exposed to loud sounds as an occupational hazard.

Another hearing disorder is Meniere's disease, a disorder of the inner ear that can lead to ringing in the ears (e.g., tinnitus like symptoms), reduction or loss in hearing, vertigo and a feeling of fullness or congestion in the ear, but which is not necessarily caused by exposure to loud noise and can be genetic in origin. Attacks of dizziness can come on suddenly or after a short period of tinnitus like symptoms or muffled hearing. Some people will have single attacks of dizziness separated by long periods of time. Others can experience many attacks closer together over a number of days. Some people with Meniere's disease have vertigo so extreme that they lose their balance and fall. Meniere's disease is often a severe and debilitating condition for afflicted patients.

There are very few options for the treatment of hearing disorders, such as tinnitus or Meniere's disease, and most available treatments are non-pharmaceutical based treatments which do not directly treat the constant ringing of the ears but are directed towards the patient's subjective management of the condition, for example, white noise machines, or counseling. There remains a need in the art for pharmaceutical compositions for reducing, ameliorating, and/or counteracting one or more symptoms of tinnitus or Meniere's disease.

### SUMMARY

Disclosed herein are pharmaceutical compositions for the treatment of hearing disorders. The present disclosure relates to a novel pharmaceutical formulation for delivering an effective dose of nimodipine (NMDP) for the treatment of a hearing disorder through transmucosal delivery, for example, intranasal delivery.

It is appreciated that mucosal delivery provides a number of advantages over conventional methods of administering pharmaceutical formulation, such as by intravenous administration, parenteral injection, or orally administered pills. For example, the patient does not have to schedule and remember to consume doses of pills or suffer painful needly sticks. The administration is not affected by stomach or digestive issues. Mucosal delivery enables drugs to avoid degradation in the gastrointestinal tract or liver. Further, mucosal delivery provides for a quick delivery of the active agent to the subject, providing rapid relief of hearing disorders which can need to be treated quickly under certain circumstances, such as if a subject is operating a vehicle, or working in a job requiring auditory perception. However, non-polar molecules, such as NMDP, are very difficult to formulate for intranasal delivery due to their low aqueous solubility, and the low volume of the intranasal cavity which limits the amount of solution which can be used to deliver the API to the subject when administered intranasally. There remains a need in the art for intranasal formulations of NMDP effective for the treatment of hearing disorders, and for providing rapid relief of symptoms of hearing disorders.

One or more aspects disclosed herein includes a pharmaceutical composition comprising a therapeutically effective amount of NMDP, composition formulated for intranasal administration, the composition comprising: a therapeutically effective amount of NMDP; and a surfactant; a solvent; and a water-soluble cellulosic polymer. One or more aspects disclosed herein includes a pharmaceutical composition comprising a therapeutically effective amount of NMDP, wherein the composition is formulated for intranasal administration, the composition comprising: a therapeutically effective amount of NMDP; and a liquid vehicle, wherein the liquid vehicle further comprises a surfactant; a water-soluble cellulosic polymer; a solvent; and water. One or more aspects disclosed herein include a pharmaceutical composition comprising a therapeutically effective amount of NMDP, composition formulated for intranasal administration, the composition comprising: a therapeutically effective amount of NMDP; a surfactant; a water-soluble cellulosic polymer; a solvent; and water. In some embodiments, the formulation further includes ethanol. In some embodiments, the ethanol is at most 5% (w/w). In some embodiments, the surfactant is a polysorbate or a combination of polysorbates. In some embodiments, the surfactant is polysorbate 20 (TWEEN-20), In some embodiments, the surfactant is polysorbate 80 (TWEEN-80). In some embodiments, the surfactant is at least 0.00001% and at most 2% (w/w). In some embodiments, the surfactant is at least 0.05% (w/w) and at most 2% (w/w). In some embodiments, the water-soluble cellulosic polymer can comprise hydroxypropyl methylcelluose (HPMC), hydroxyethyl cellulose (HEC) HEC, carboxymethyl cellulose (CMC) and its sodium salt (CMC Na), or a combination thereof. In some embodiments, the water-soluble cellulosic polymer is at least 0.00001% and at most 2% (w/w). In some embodiments, the water-soluble cellulosic polymer is at least 0.05% (w/w) and at most 2% (w/w). In some embodiments, the solvent is PEG, an alkoxy derivative of polyethylene glycol, methoxy polyethylene glycol (mPEG), mPEG350, polyethylene glycol (PEG400), or a combination thereof. In some embodiments, the solvent is at least 0.00001% and at most 94% (w/w). In some embodiments, the solvent is at least 66% (w/w) and at most 94% (w/w). In some embodiments, the water is at least 0.0% and at most 50% (w/w). In some embodiments, the water is at least 0.0% and at most 20% (w/w). In some embodiments, the water is at least 0.00001% (w/w) and at most 50% (w/w). In some embodiments, the water is at least 0.0001% (w/w) and at most 20% (w/w). In some embodiments, the surfactant is a non-ionic surfactant. In some embodiments, the non-ionic surfactant is a polysorbate or a combination of polysorbates. In some embodiments, the surfactant is polysorbate 20 (Tween-20). In some embodiments, the surfactant is polysorbate 80 (Tween-80). In some embodiments, the polysorbate 80 (Tween-80) is at least 0.00001% and at most 2% (w/w). In some embodiments, the polysorbate 80 (Tween-80) is at least 0.05% (w/w) and at most 2% (w/w). In yet other embodiments, the Tween-80 is less than about 2.5%. In some embodiments, the water-soluble cellulosic polymer is HPMC, HEC, CMC, carboxymethylcellulose sodium, or a combination thereof. In some embodiments, the water-soluble cellulosic polymer only includes HPMC. In some embodiments, the HPMC is at least 0.00001% and at most 2% (w/w). In some embodiments, the HPMC is at least 0.05% (w/w) and at most 2% (w/w). In some embodiments, the solvent is PEG, an alkoxy derivative of polyethylene glycol, mPEG, mPEG350, PEG400 or a combination thereof. In some embodiments, the PEG, an alkoxy derivative of polyethylene glycol, mPEG, mPEG350, PEG400, or a combination thereof is at least 0.0% and at most 94% (w/w). In some embodiments, the PEG, the alkoxy derivative of polyethylene glycol, mPEG, mPEG350, PEG400 or a combination thereof is at least 66% (w/w) and at most 94% (w/w). In some embodiments, the water is at least 0.00001% and at most 20% (w/w). In some embodiments, the water is at least 0.00% (w/w) and at most 20% (w/w).

One or more aspects disclosed herein includes a process for preparing an NMDP liquid composition, the process comprising the steps of: dissolving Tween 80 (<2% w/w) separately in mPEG350 and PEG400; mixing NMDP with the solutions from Step 1) to saturation solubility, or a desired effective concentration; preparing an aqueous solution of HPMC or HPMC in buffer (<2% w/w) with a pH value in the range of about 6.4 to about 7.4; and adding an aqueous HPMC solution or HPMC buffer from Step 3) to the respective solutions from Step 2) with agitation and allow the mixtures to stand to observe solution or suspension stability. In some embodiments, the Step 2) solution is NMDP in a PEG/Tween 80 solution or NMDP in a mPEG/Tween 80 solution. In some embodiments, the concentration of NMDP is at least 68 mg/mL. In some embodiments, the concentration of NMDP is at least 0.1 mg/mL up to 100 mg/mL. In some embodiments, the concentration of NMDP is 68 mg/mL. In some embodiments, the concentration of NMDP is at least about 1, 2, 3, 4, 5, 6, 7, 8, 9, 10, 15, 20, 25, 30, 35, 40, or 45 mg/mL.

In some embodiments, the concentration of NMDP is 50 mg/mL. In some embodiments, the surfactant, water-soluble cellulosic polymer, solvent, water and ethanol comprise a liquid vehicle. In some embodiments, the liquid vehicle is less than 1000, 900, 800, 700, 600, 500, or 400 μL. In some embodiments, the liquid vehicle is less than 300 μL. In some embodiments, the liquid vehicle is less than 150 μL. In some embodiments, the NMDP is dissolved, suspended, or both dissolved and suspended in the liquid vehicle.

Aspects disclosed herein provide a method of treating neural disorders such as hearing disorders, including tinnitus or Meniere's disease or a symptom of tinnitus or Meniere's disease in a subject in need thereof comprising intranasally administering a therapeutically effective amount of NMDP or a salt thereof.

In some embodiments, the therapeutically effective amount of NMDP is from about 0.1 mg to about 5 mg per kg of the subject. In some embodiments, the method further comprises administering the pharmaceutical composition in a volume from about 10 μl to about 300 μl per dose. In some embodiments, the method further comprises bringing at least a portion of the therapeutically effective amount of the pharmaceutical composition into contact with the mucosal membrane of at least one nasal cavity. In some embodiments, the method further comprises spraying a first quantity of the pharmaceutical composition into the first nasal cavity, spraying a second quantity of the pharmaceutical composition into a second nasal cavity, and optionally after a pre-selected time delay, spraying a third quantity of the pharmaceutical composition into the first nasal cavity. In some embodiments, the method further comprises optionally after a pre-selected time delay, administering at least a fourth quantity of the pharmaceutical composition to the second nostril. In some embodiments, said method of treatment achieves bioavailability that is from about 80%-125% of that achieved with the same pharmaceutical composition administered intravenously. In some embodiments, the method further comprises administering the pharmaceutical composition at any time before or after onset of symptoms of tinnitus or Meniere's disease. In some embodiments, the method further comprises administering the pharmaceutical composition at least once a day, twice a day, three times a day, four times a day, five times a day, six times a day, seven times a day, eight times a day, nine times a day, or ten times a day. In some embodiments, the therapeutically effective amount of NMDP is administered intranasally every 2 hours. In some embodiments, the therapeutically effective amount of NMDP is administered intranasally every 4 hours. In some embodiments, the method further comprises administering the pharmaceutical composition weekly. In some embodiments, the method further comprises administering the pharmaceutical composition via a low-dose therapy. In some embodiments, the method further comprises administering the pharmaceutical composition by titration to vestibular symptoms. In some embodiments, the method further comprises administering the pharmaceutical composition for at least 1, 2, 3, 4, 5, 6, 14, 21, 28, 60, 120, or 400 days. In some embodiments, the method further comprises administering the pharmaceutical composition in a unit dosage form. In some embodiments, the method further comprises administering the pharmaceutical composition in order to not cause a change in cochlear potential. In some embodiments, the method further comprises increasing the subject's dose of the pharmaceutical composition until a symptom of inner ear disturbance is observed. In some embodiments, the symptom of inner ear disturbance comprises spontaneous nystagmus observed with Frenzel's glasses, disequilibrium, motion intolerance, or reduction/loss in hearing. In some embodiments, the method further comprises administering the pharmaceutical composition via drug delivery device. In some embodiments, the effect of the pharmaceutical composition is measured by an assessment selected from the group consisting of a) measurement of a change in auditory brainstem response (ABR) threshold, b) a change in auditory speech recognition as measured by a words-in-noise test, c) a change in auditory speech recognition as measured by a digits-in-noise test, d) a change in low-frequency hearing threshold e) a change in incidence of adverse events after administration of the pharmaceutical composition, f) a change in tinnitus or Meniere's disease severity g) a change in tinnitus or Meniere's disease loudness h) a change in vertigo severity i) a change in aural fullness j) a change in dizziness, and k) a change in hair cell function as observed when measured by a change in ABR threshold, after administration of the pharmaceutical composition. In some embodiments, the measurement of the ABR threshold is performed at the frequency range of 200 Hz-30 kHz.

Aspects disclosed herein provide a method of treating tinnitus or Meniere's disease or a symptom of tinnitus or Meniere's disease in a subject in need thereof comprising: administering an L-type calcium channel blocker to the subject; assessing if the subject was responsive to the L-type calcium channel blocker; and administering a therapeutically effective amount of NMDP or a salt thereof to the subject if the subject was responsive to the L-type calcium channel blocker.

In some embodiments, the method further comprises intranasally administering the therapeutically effective amount of NMDP or a salt thereof to the patient for treatment of tinnitus or Meniere's disease or a symptom of tinnitus or Meniere's disease. In some embodiments, the therapeutically effective amount of NMDP is administered in a composition comprising: the therapeutically effective amount of NMDP; a carrier; a citrate buffer; and benzalkonium chloride. In some embodiments, the therapeutically effective amount of NMDP is administered in a composition comprising: the therapeutically effective amount of NMDP; one or more natural or synthetic carriers, or any combinations thereof, in an amount from about 30% to about 95% (w/w); and a non-aqueous solvent from about 10% to about 70% (w/w); and a surfactant. In some embodiments, the therapeutically effective amount of NMDP is administered in a composition that comprises at least one of: PEG, mPEG, or water. In some embodiments, the therapeutically effective amount of NMDP is administered in a composition that comprises PEG, mPEG, and water. In some embodiments, the therapeutically effective amount of NMDP is administered in a composition that comprises about 20% to about 90% (w/w) mPEG, about 10% to about 50% (w/w) PEG, and about 5% to about 20% (w/w) water and NMDP. In some embodiments, the therapeutically effective amount of NMDP is administered in a composition that comprises about 70% (w/w) mPEG, about 20% (w/w) PEG, and about 10% (w/w) water.

Aspects disclosed herein provide a method of achieving a therapeutically effective area under the curve (AUC) extrapolated to infinity from dosing time (AUC0-infinity) of NMDP in a subject in need thereof, comprising intranasally administering an intranasal pharmaceutical composition to the subject, wherein the intranasal pharmaceutical composition comprises: from about 0.1 mg to about 5 mg/kg NMDP, or a pharmaceutically acceptable salt thereof; a buffer; and a surfactant, wherein the subject exhibits an AUC0-infinity of NMDP which is between about 200 h*ng/mL and 400 h*ng/ml following administration of the intranasal pharmaceutical composition to the subject.

One aspect disclosed herein provides a pharmaceutical composition comprising a therapeutically effective amount of NMDP, composition formulated for intranasal administration, the composition comprising: a therapeutically effective amount of NMDP; a buffer; a penetration enhancer; and a surfactant.

In some embodiments, the composition further comprises one or more of the ingredients selected from vitamin E, benzyl alcohol, and dodecyl maltoside. In some embodiments, the concentration of NMDP in the pharmaceutical composition administered is 0.5, 1, 1.5, 2, 2.5, 3, 3.5, 4, 4.5, 5, 5.5, 6, 6.5, 7, 7.5, 8, 8.5, 9, 9.5, 10, 12.5, 15, 17.5, 20, 22.5, 25, 27.5, 30, 32.5, 35, 37.5, 40, 42.5, 45, 47.5, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, or 400 mg/ml. In some embodiments, the volume of the pharmaceutical composition administered is 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1.0 ml. In some embodiments, the pharmaceutical composition is an extended-release formulation. In some embodiments, the pharmaceutical composition is a sustained release formulation. In some embodiments, the pharmaceutical composition is a controlled release formulation. In some embodiments, the pharmaceutical composition is released either continuously, variably, or in a pulsatile manner, or combinations thereof. In some embodiments, the pharmaceutical composition is in a unit dosage form. In some embodiments, the unit dosage form is in a dry powder, semi-solid, a mucoadhesive formulation, intranasal vesicular unit or solution dosage form. In some embodiments, the composition is aqueous. In some embodiments, the composition is in the form of gel or film. In some embodiments, the composition comprises micronized particles. In some embodiments, the unit dosage form has a unit weight of from about 10 mg to about 10 g. In some embodiments, the unit dosage form has a unit weight of from about 10 mg to about 50 mg, about 10 mg to about 100 mg, about 10 mg to about 150 mg, about 10 mg to about 300 mg, about 10 mg to about 500 mg, about 10 mg to about 1 g, or about 10 mg to about 5 g. In some embodiments, the solution dosage form has a unit dosage volume of less than about 900, 800, 700,600, 500, 400, 300, 200, or 100 μL. In some embodiments, the pharmaceutical composition comprising NMDP has a concentration of from about 0.1% to about 20% w/w of the formulation. In some embodiments, the pharmaceutical composition prolongs the residence time of the composition in an auris structure. In some embodiments, the formulation prolongs the residence time of the composition in the auris structures for a period of at least 1 day, at least 2 days, at least 3 days, at least 4 days, at least 5 days, at least 6 days, at least 8 days, or at least 14 days, at least 21 days, or at least 1 month, or at least 6 weeks after a single administration. In some embodiments, the formulation increases the bioavailability of the composition in the auris structure. In some embodiments, the formulation increases the steady state levels of the composition in the auris structure. In some embodiments, the formulation increases the time to reach Cmax of the therapeutic concentration that is capable of reducing the symptom of the hearing disorder in the subject in need thereof. In some embodiments, the formulation prolongs the time that the concentration of the composition will stay above the minimum therapeutic concentration (i.e., Cmin) necessary for reducing the symptom of the hearing disorder in the subject in need thereof. In some embodiments, the concentration of the composition in the auris structures stays at or about concentrations greater than Cmin for a period of at least 3 days, 4 days, 5 days, 6 days, 7 days, 10 days, 14 days, 3 weeks or 1 month. In some embodiments, the pharmaceutical composition further comprises a second pharmaceutical active agent.

Aspects disclosed herein provide a pharmaceutical composition comprising a therapeutically effective amount of NMDP, wherein composition is formulated for intranasal administration, the composition comprising:

a therapeutically effective amount of NMDP; a solvent; and water. In some embodiments, the solvent is PEG, an alkoxy derivative of polyethylene glycol, mPEG, mPEG350, PEG400, or a combination thereof. In some embodiments, the solvent comprises at least 0.00001% and at most 94% (w/w) of the composition. In some embodiments, the solvent comprises at least 66% (w/w) and at most 94% (w/w) of the composition. In some embodiments, the water comprises the water at least 0.00001% and at most 20% (w/w) of the composition. In some embodiments, the composition further comprises a first solvent and a second solvent. In some embodiments, the first solvent comprises mPEG and the second solvent comprises PEG. In some embodiments, the mPEG comprises mPEG350. In some embodiments, the PEG comprises PEG400. In some embodiments, the therapeutically effective amount of NMDP comprises at least 10 mg/mL, at least 15 mg/mL, at least 20 mg/mL, at least 25 mg/mL, at least 30 mg/mL, at least 35 mg/mL, at least 40 mg/mL, at least 45 mg/mL, at least 50 mg/mL, at least 55 mg/mL, at least 60 mg/mL, at least 65 mg/mL, at least 70 mg/mL, at least 80 mg/mL, at least 90 mg/mL, at least 95 mg/mL, at least 100 mg/mL, at least 110 mg/mL, or greater than 110 mg/mL. In some embodiments, the mPEG comprises about 20% to about 90%, about 30% to about 90%, about 40% to about 90%, about 50% to about 90%, about 60% to about 90%, about 70% to about 90% or about 80% to about 90% (w/w) of the composition, the PEG comprises about 10% to about 50%, about 20% to about 50%, about 30% to about 50%, or about 40% to about 50% (w/w) of the composition, and the water comprises about 5% to about 20% about 10% to about 20%, or about 15% to about 20% (w/w) of the composition. In some embodiments, the composition further comprises ethanol. In some embodiments, the ethanol comprises about 1% to about 10% about 2% to about 10%, about 3% to about 10%, about 4% to about 10%, about 5% to about 10%, about 6% to about 10%, about 7% to about 10%, about 8% to about 10%, or about 9% to about 10% of the composition.

These and other objects and features of the disclosure will be more fully appreciated when the following detailed description of the disclosure is read in conjunction with the accompanying examples and drawings.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows results of a no-go trial indicating the efficacy of treating tinnitus with a drug of interest at varying dosages, as compared to subjects treated with saline;

FIG. 1B shows results of a go trial indicating the subject's motivation, hearing, and learning memory, and serves as a control for the no-go trial with respect to the effects of the drug on the subject's behavior;

FIG. 2A a chart summarizing the results of the indicating the subject's motivation, hearing, and learning memory, and serves as a control for the no-go trial with respect to the effects of the drug on the subject's behavior;

FIG. 2B shows a graph summarizing the results of the indicating the subject's motivation, hearing, and learning memory, and serves as a control for the no-go trial with respect to the effects of the drug on the subject's behavior;

FIG. 2C shows a chart summarizing the results of the no-go trial indicating the efficacy of treating tinnitus or tinnitus like symptoms with a drug of interest at varying dosages, as compared to subjects treated with saline;

FIG. 2D shows a graph summarizing the results of the no-go trial indicating the efficacy of treating tinnitus or tinnitus like symptoms with a drug of interest at varying dosages, as compared to subjects treated with saline;

FIG. 3 summarizes the go/no-go trial sound based avoidance detection methodology utilized herein.

### DETAILED DESCRIPTION

NMDP is a calcium channel blocker (CCB) belonging to the dihydropyridine class and is a highly lipophilic agent that rapidly crosses the blood-brain barrier.

It is difficult to formulate NMDP for intranasal delivery because of its high lipophilicity including its low hydrogen bond donor count and high hydrogen bond acceptor count. The NMDP molecule doesn't have many hydroxyl groups. Instead, NMDP has many carbonyl groups which do not provide the electron donating pairs to form hydrogen bonds and decrease its water solubility.

Furthermore, to facilitate intranasal administration of NMDP, an effective amount of NMDP should be dissolved or finely dispersed in a small volume of liquid vehicle. Larger volumes drain out anteriorly through the nostrils or posteriorly toward the pharynx where excess liquid is swallowed. As a result, if large volumes are administered, a portion of NMDP can be lost from the absorption site, and it can be difficult if not impossible to reproducibly administer the correct dose of the therapeutic agent. Thus, it is desirable to have a high dissolved NMDP concentration or NMDP content dispersed in a small volume of liquid vehicle for intranasal administration.

Furthermore, to support drug absorption through the nasal mucosa, two natural protective functions must be bypassed—the mucociliary clearance (MCC) and the barrier properties of the tissue. It is desirable that an effective dosage can bypass natural attributes of the mucus blanket as a protective layer, i.e., increase drug absorption, and of the MCC as an effective cleansing mechanism. In other words, it is desirable to increase resident time of the applied dose on the mucous layer.

Responsive to the unmet need in the art for pharmaceutical compositions for treating hearing disorders, disclosed herein are pharmaceutical compositions for the treatment of hearing disorders, for example, tinnitus or Meniere's disease. The present disclosure relates to novel pharmaceutical formulations for delivering an effective dose of NMDP for the treatment of a hearing disorder through transmucosal delivery, for example, intranasal delivery.

It is appreciated that mucosal delivery provides a number of advantages over conventional methods of administering pharmaceutical formulation, such as by intravenous administration, injection, or orally administration. For example, the patient does not have to schedule and remember to consume doses of pills, or suffer painful needly sticks. The administration is not affected by stomach or digestive issues. Mucosal delivery enables drugs to avoid degradation in the gastrointestinal tract or liver. Mucosal delivery is therefore of particular interest for molecules with limited systemic bio-availabilities and short half-lives, such as NMDP. Further, mucosal delivery provides for a quick delivery of the active agent to the subject, providing rapid relief of hearing disorders, such as tinnitus or Meniere's disease.

Mucosal administration, such as, intranasal, buccal, sublingual, rectal and pulmonal administration, is receiving particular interest as it avoids many of the disadvantages of injecting a therapeutic agent while, at the same time, still providing a strong and rapid systemic effect. In order to be an attractive alternative to injection, mucosal administration, for example, intranasal administration, should neither cause significant pain, discomfort or irritation nor cause any irreversible damage to the mucosal surface. However, in the case of acute health threatening indications, a relatively high local irritation to the mucosa can be acceptable.

In mucosal administration, such as during nasal, buccal or rectal administration, the therapeutic agent should be applied to the mucosa in a vehicle that permits it to penetrate, or be absorbed through, the mucosa. In order to penetrate the mucus, the vehicle should be biocompatible with mucus and hence have a certain degree of hydrophilicity. However, the vehicle should preferably also possess lipophilic properties to dissolve a clinically relevant amount of the therapeutic agent of interest.

The extensive network of blood capillaries under the mucosal surface, especially in the nasal mucosa, is well suited to provide a rapid and effective systemic absorption of drugs, vaccines and biologicals. Moreover, the nasal epithelial membrane in effect contains a single layer of epithelial cells (pseudostratified epithelium) and, therefore, is more suited for drug administration than other mucosal surfaces having squamous epithelial layers, such as, the mouth and vagina.

Intranasal delivery has many advantages over other routes of administration, namely its non-invasiveness, rapid attainment of therapeutically relevant concentrations to the bloodstream, no first-pass metabolism, and ease of administration. Viable nasal delivery technologies have the potential to enable drug developers in creating innovative medicines using already approved products by delivering them through new routes of administration. Intravenous administration of NMDP has several disadvantages compared to transmucosal administration, such as increased side effects. However, non-polar molecules, such as NMDP, are very difficult to formulate for intranasal delivery due to their low aqueous solubility, and the low volume of the intranasal cavity which limits the amount of solution which can be used to deliver the API to the subject when administered intranasally. There remains a need in the art for intranasal formulations of NMDP effective for the treatment of hearing disorders, such as tinnitus or Meniere's disease, and for providing rapid relief of symptoms of hearing disorders.

The present investigators have developed a composition which administers by transmucosal delivery an effective dosage of an equivalent of NMDP to alleviate the symptoms of tinnitus or Meniere's disease after the administration of one or more doses of the pharmaceutical composition.

### Definitions

Unless defined otherwise, all terms of art, notations and other technical and scientific terms or terminology used herein are intended to have the same meaning as is commonly understood by one of ordinary skill in the art to which the claimed subject matter pertains. In some embodiments, terms with commonly understood meanings are defined herein for clarity and/or for ready reference, and the inclusion of such definitions herein should not necessarily be construed to represent a substantial difference over what is generally understood in the art.

Throughout this application, various embodiments can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject can be diagnosed or suspected of being at high risk for a disease. In some embodiments, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit can refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject can still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease cannot have been made.

The term “NMDP” is intended to relate to NMDP or a pharmaceutically acceptable salt thereof. The term “equivalent to about . . . of NMDP” is intended to relate to a specified volume, concentration, or amount of NMDP free base provided by a volume, concentration, or amount of a salt of NMDP. Thus, the specified amount relates to the amount of NMDP free base and not the amount of the NMDP salt, despite the use of the salt in the composition. In an embodiment, the composition, methods and uses of the present disclosure comprise the use of NMDP citrate.

The term “formulated” is intended to relate to the selection of excipients, carriers, vehicles, preservatives, stabilizing agents and so forth in the preparation of medicament using said composition. The term “formulated” is furthermore intended to relate to the selection of the device for delivery of the composition or selection of containment device for administration or storing of the composition.

The term “dosage unit” relates to the composition administered in one administration by one delivery operation. In the embodiment wherein the composition is formulated for transmucosal administration by nasal delivery, a dosage unit is the volume of the composition administered or amount of agent administered by one delivery operation. A delivery operation is an operation which delivers a dosage unit. In this embodiment, a delivery operation is the administration to the nasal cavity of a dosage unit by means of a delivery system, such as a nasal spray or other means known to the person skilled in the art. Suitable devices are commercially available from e.g., Pfeiffer and Valois. The terms “dosage” and “treatment dosage” relates to the total amount of agent or volume of composition applied by means of administration of dosage units during a treatment. A treatment relates to the administration of the composition during a single episode of a hearing disorder, including tinnitus or Meniere's disease or a symptom of tinnitus or Meniere's disease, said episode lasting until alleviation of tinnitus or Meniere's disease or a symptom of tinnitus or Meniere's disease.

The term “time-to-onset-of-action” is intended to mean the moment wherein the patient begins to experience tinnitus or Meniere's disease or a symptom of tinnitus or Meniere's disease relief, usually as a result of sufficient plasma concentrations of NMDP. Sufficient plasma concentrations to achieve all varies amongst patients, amongst patient classes and types and nature of tinnitus or Meniere's disease or a symptom of tinnitus or Meniere's disease experienced. The “action” in “time-to-onset-of-action” is tinnitus or Meniere's disease or a symptom of tinnitus or Meniere's disease relief.

The term “duration-of-action” relates to the time throughout which tinnitus or Meniere's disease or a symptom of tinnitus or Meniere's disease relief is experienced by the patient.

An “extended time period” intends a period of more than about 4 months, preferably more than about 6 months.

The phrase “amount effective to suppress one or more symptoms of tinnitus or Meniere's disease” refers to a dose of the therapeutic compound, that reduces one or more symptoms of tinnitus or Meniere's disease are alleviated in a subject after delivery of the pharmaceutical composition.

The terminology used herein is for describing particular embodiments only and is not intended to be limiting of the disclosure. The term “comprising” and “comprises”, used in the claims, should not be interpreted as being restricted to the components and steps listed thereafter; they do not exclude other components or steps. They need to be interpreted as specifying the presence of the stated features, integers, steps and/or components as referred to, but does not preclude the presence and/or addition of one or more other features, integers, steps or components, or groups thereof. Thus, the scope of the expression “a composition comprising A and B” should not be limited to compositions consisting only of components A and B. Also, the scope of the expression “a method comprising the steps X and Z” should not be limited to methods consisting exclusively of those steps.

As used herein, the term “about” a number refers to that number plus or minus 10% of that number. The term “about” a range refers to that range minus 10% of its lowest value and plus 10% of its greatest value. For example, the term “about” can be immediately understood as within 10%, 9%, 8%, 7%, 6%, 5%, 4%, 3%, 2%, 1%, 0.5%, 0.1%, 0.05%, or 0.01% of the stated value. As an illustration, a numerical range of “about 1 to about 5” should be interpreted to include not only the explicitly recited values of about 1 to about 5, but also include individual values and sub-ranges within the indicated range. This includes, at very least, the degree of expected experimental error, technical error and instrumental error for a given experiment, technique or an instrument used to measure a value.

As used herein, the term “and/or” includes any combinations of one or more of the associated listed items. Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached to”, “connected to”, “coupled with”, “contacting”, etc., another element, it can be directly on, attached to, connected to, coupled with or contacting the other element or intervening elements can also be present. In contrast, when an element is referred to as being, for example, “directly on”, “directly attached to”, “directly connected to”, “directly coupled” with or “directly contacting” another element, there are no intervening elements present. It will also be appreciated by those of skill in the art that references to a structure or feature that is disposed “adjacent” another feature can have portions that overlap or underlie the adjacent feature.

The terms “active agent”, “pharmaceutical active agent”, “active”, “API”, “active pharmaceutical ingredient”, “active substance”, “active molecule”, “active compound” or “drug” are used interchangeably with NMDP or a salt thereof.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The disaggregating agent comprised in the disclosed composition is an inert carrier, as described in detail below. It is to be noted that “disaggregating agent”, “carrier”, “diluent” and “deagglomerating agent” are used herein interchangeably, and refer to an inert ingredient added to the pharmaceutical composition, comprising the said second type particles.

“Mucosal delivery enhancing agents” are defined as chemicals and other excipients that, when added to a formulation comprising water, salts and/or common buffers and NMDP (the control formulation) produce a formulation that results in a significant increase in transport of a NMDP across a mucosa as measured by the maximum blood, serum, or cerebral spinal fluid concentration (Cₘₐₓ) or by the area under the curve (AUC) in a plot of concentration versus time. A mucosa includes the nasal, oral, intestinal, buccal, bronchopulmonary, vaginal, and rectal mucosal surfaces and includes all mucus-secreting membranes lining all body cavities or passages that communicate with the exterior. Mucosal delivery enhancing agents are sometimes called carriers, excipients, additives, enhancing agents or enhancers (including, for example, a thickening agent).

“Endotoxin-free formulation” means a formulation comprising n NMDP and one or more mucosal delivery enhancing agents that is substantially free of endotoxins and/or related pyrogenic substances. Endotoxins include toxins that are confined inside a microorganism and are released only when the microorganisms are broken down or die. Pyrogenic substances include fever-inducing, thermostable substances (glycoproteins) from the outer membrane of bacteria and other microorganisms. These substances can cause fever, hypotension and shock if administered to humans. Producing formulations that are endotoxin-free can require special equipment, expert artisans, and can be significantly more expensive than making formulations that are not endotoxin-free.

“Non-infused administration” means any method of delivery that does not involve an injection directly into an artery or vein, a method which forces or drives (typically a fluid) into something and especially to introduce into a body part by means of a needle, syringe or other invasive method. Non-infused administration includes subcutaneous injection, intramuscular injection, intraperitoneal injection and the non-injection methods of delivery to a mucosa.

A “subject” in accordance with some embodiments is a subject in which the symptoms and symptoms, the physical examination results and/or the psychological test results are determined and recorded in relation to the condition of the individual (i.e., the disease or disorder condition). As used herein, the disease or disorder is a hearing disorder. As used herein, the disease or disorder is tinnitus or Meniere's disease. As used herein, “subject” is intended to be a human subject, but is not necessarily limited thereto. The subject can be male or female and can be any race or ethnic group including, but not limited to, Caucasian, African American, African, Asian, Hispanic, Indians and the like. As used herein, a subject is an animal, particularly a mammal such as a dog, cat, cow, goat, horse, sheep, or both, that can be treated according to the methods of the disclosure or screened for veterinary and pharmaceutical or pharmaceutical drug development purposes, Pigs, rodents (e.g., rats and mice), rabbit necks, primates (including non-human primates), and the like. A subject according to some embodiments of the present disclosure includes a patient or a human in need of a therapeutic treatment for a disorder treatable by

### NMDP Compositions

The pharmaceutical composition of the disclosure comprises NMDP, or salts thereof, in a suitable solvent at a concentration equivalent to about 0.4 to 75 mg/mL of NMDP. In some embodiments, the concentration equivalent of NMDP can be greater than 75 mg/mL. In some embodiments, the concentration equivalent of NMDP can be 25 mg/mL. In some embodiments, the concentration equivalent of NMDP can be 50 mg/mL. In some embodiments, the concentration equivalent of NMDP can be 75 mg/mL. In some embodiments, the concentration equivalent of NMDP can be 100 mg/mL. The composition is suitably formulated for transmucosal administration, typically to deliver NMDP through the nasal mucosa.

The therapeutically effective amount of NMDP or a salt thereof can be at least 1 μg of NMDP or a salt thereof per kg of the subject. The therapeutically effective amount of NMDP or a salt thereof can also be less than 100 mg of NMDP or a salt thereof per kg of the subject. The therapeutically effective amount of NMDP or a salt thereof ranges from 1 μg to 1000 mg of NMDP or a salt thereof per kg of the subject. In some embodiments, the therapeutically effective amount of NMDP or a salt thereof ranges from 16 mg to 24 mg of NMDP or a salt thereof per kg of the subject. In some embodiments, the therapeutically effective amount of NMDP or a salt thereof ranges from 30 mg to 100 mg of NMDP or a salt thereof per kg of the subject. In some embodiments, the therapeutically effective amount of NMDP or a salt thereof ranges from 30 mg to 100 mg of NMDP or a salt thereof per kg of the subject.

The therapeutically effective amount of NMDP or a salt thereof can be administered before occurrence of the hearing disorders, such as, for tinnitus or Meniere's disease. The therapeutically effective amount of NMDP or a salt thereof can be administered at least about 1 hour before occurrence of the hearing disorders, for example, at least about 1 hour, 2 hours, 3 hours, 4 hours, 5 hours, 6 hours, 12 hours, 24 hours, 36 hours, 48 hours, 3 days, 4, days, 5 days, 6 days, 7 days, 2 weeks, 3 weeks, or 1 month before occurrence of the hearing disorder. The therapeutically effective amount of NMDP or a salt thereof can be administered less than about 1 month before occurrence of the hearing loss, for example, less than about 1 hour, 2 hours, 3 hours, 4 hours, 5 hours, 6 hours, 12 hours, 24 hours, 36 hours, 48 hours, 3 days, 4, days, 5 days, 6 days, 7 days, 2 weeks, 3 weeks, or 1 month before occurrence of the hearing disorders. In some embodiments, the therapeutically effective amount of NMDP or a salt thereof is administered at least 12 hours before occurrence of the hearing disorder. In some embodiments, the therapeutically effective amount of NMDP or a salt thereof is administered less than 10 days before occurrence of the hearing disorders. In some embodiments, the therapeutically effective amount of NMDP or a salt thereof is administered between 12 hours to 10 days before occurrence of the hearing disorders. In some embodiments, the therapeutically effective amount of NMDP or a salt thereof is administered between 12 hours to 48 hours before occurrence of the hearing disorders.

The therapeutically effective amount of NMDP or a salt thereof can be administered after or contemporaneously upon occurrence of the hearing disorders, such as, for tinnitus or Meniere's disease. The therapeutically effective amount of NMDP or a salt thereof can be administered at least about 1 minute after occurrence of the hearing disorders, for example, at least about 1 minute, 2 minutes, 3 minutes, 4 minutes, 5 minutes, 6 minutes, 12 minutes, 24 minutes, 36 minutes, 48 minutes, 54 minutes, or 60 minutes after occurrence of the hearing disorder. The therapeutically effective amount of NMDP or a salt thereof can be administered at least about 1 hour after occurrence of the hearing disorders, for example, at least about 1 hour, 2 hours, 3 hours, 4 hours, 5 hours, 6 hours, 12 hours, 24 hours, 36 hours, 48 hours, 3 days, 4, days, 5 days, 6 days, 7 days, 2 weeks, 3 weeks, or 1 month after occurrence of the hearing disorder. The therapeutically effective amount of NMDP or a salt thereof can be administered less than about 1 month after occurrence of the hearing loss, for example, less than about 1 hour, 2 hours, 3 hours, 4 hours, 5 hours, 6 hours, 12 hours, 24 hours, 36 hours, 48 hours, 3 days, 4, days, 5 days, 6 days, 7 days, 2 weeks, 3 weeks, or 1 month after occurrence of the hearing disorders. In some embodiments, the therapeutically effective amount of NMDP or a salt thereof is administered at least 12 hours after occurrence of the hearing disorder. In some embodiments, the therapeutically effective amount of NMDP or a salt thereof is administered less than 10 days after occurrence of the hearing disorders. In some embodiments, the therapeutically effective amount of NMDP or a salt thereof is administered between 12 hours to 10 days after occurrence of the hearing disorders. In some embodiments, the therapeutically effective amount of NMDP or a salt thereof is administered between 12 hours to 48 hours after occurrence of the hearing disorders.

### Effective Dosage Ranges

The therapeutically effective amount of NMDP can be dependent on the weight of a subject. In some embodiments, the therapeutically effective amount of NMDP is at least about 1 mg of NMDP per kg of the subject, for example at least about 1, 2, 3, 4, 5, 6, 7, 8, 9, 10, 20, 30, 40, 50, 60, 70, 80, 90, 100, 200, mg of NMDP per kg of the subject. In some embodiments, the therapeutically effective amount of NMDP is less than about 1000 μg of NMDP per kg of the subject, for example less than about 1, 2, 3, 4, 5, 6, 7, 8, 9, 10, 20, 30, 40, 50, 60, 70, 80, 90, 100, 200, 300, 400, 500, 600, 700, 800, 900 or 1000 μg of NMDP per kg of the subject. In some embodiments, the therapeutically effective amount of NMDP ranges from about 1 μg to 1000 μg of NMDP per kg of the subject, for example about 1-700, 1-500, 1-300, 1-100, 1-50, 1-10, 10-700, 10-500, 10-300, 10-100, 10-80, 10-60, 10-40, 10-20, 50-700, 50-500, 50-300, 50-100, 100-700, 100-500, 100-300, 300-700, 300-500, or 500-700 μg of NMDP per kg of the subject. In some embodiments, the therapeutically effective amount of NMDP ranges from about 1 μg to 10 μg of NMDP per kg of the subject. In some embodiments, the therapeutically effective amount of NMDP ranges from about 10 μg to 100 μg of NMDP per kg of the subject. In some embodiments, the therapeutically effective amount of NMDP ranges from about 100 μg to 500 μg of NMDP per kg of the subject. In some embodiments, the therapeutically effective amount of NMDP ranges from about 1 μg to 1000 mg of NMDP per kg of the subject, for example about 1-700, 1-500, 1-300, 1-100, 1-50, 1-10, 10-700, 10-500, 10-300, 10-100, 10-80, 10-60, 10-40, 10-20, 50-700, 50-500, 50-300, 50-100, 100-700, 100-500, 100-300, 300-700, 300-500, or 500-700 mg of NMDP per kg of the subject. In some embodiments, the therapeutically effective amount of NMDP ranges from about 16 mg to 24 mg of NMDP per kg of the subject. In some embodiments, the therapeutically effective amount of NMDP ranges from about 30 mg to 100 mg of NMDP per kg of the subject. In some embodiments, the therapeutically effective amount of NMDP ranges from about 50 mg to 140 mg of NMDP per kg of the subject. In some embodiments, the therapeutically effective amount of NMDP ranges from about 115 mg to 125 mg of NMDP per kg of the subject. The therapeutically effective amount of NMDP can also be the daily dosage of NMDP for the subject.

In pharmaceutical compositions with the therapeutically effective amount of NMDP and one or more API, the amount of API in the pharmaceutical composition can be dependent on the weight of a subject. In some embodiments, the amount of API in the pharmaceutical composition is at least about 1 mg of API per kg of the subject, for example at least about 1, 2, 3, 4, 5, 6, 7, 8, 9, 10, 20, 30, 40, 50, 60, 70, 80, 90, 100, 200, mg of API per kg of the subject. In some embodiments, the amount of API in the pharmaceutical composition is less than about 1 mg of API per kg of the subject, for example less than about 1, 2, 3, 4, 5, 6, 7, 8, 9, 10, 20, 30, 40, 50, 60, 70, 80, 90, 100, 200 mg of API per kg of the subject. In some embodiments, the amount of API in the pharmaceutical composition ranges from about 1-200 mg of API per kg of the subject. In some embodiments, the therapeutically effective amount of API ranges from about 30 mg to 100 mg of API per kg of the subject. In some embodiments, the therapeutically effective amount of API ranges from about 50 mg to 140 mg of API kg of the subject. The therapeutically effective amount of API can also be the daily dosage of API for the subject. For instance, the daily dosage of sodium channel blockers, the antioxidants, NMDA antagonists, SSRI or combined SSRI/NMDA antagonists can be about 1 to 500 mg/day, preferably 4 to 250 mg/day.

The concentration of NMDP in pharmaceutical compositions can be at least about 0.1%, e.g., at least about 0.1%, 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, or 90% by weight. The concentration of NMDP in pharmaceutical compositions can be less than about 99%, e.g., less than about 10%, 15%, 20%, 25%, 30%, 35%, 40%, 45%, 50%, 55%, 60%, 65%, 70%, 75%, 80%, 85%, 90% or 99% by weight. The concentration of NMDP in pharmaceutical compositions can range from about 0.1 to 99%, e.g., 0.1 to 0.5%, 0.1 to 1%, 0.5 to 1%, 1 to 2%, 1 to 5%, 1 to 10%, 2 to 5%, 2 to 10%, 5 to 10%, 10 to 15%, 15 to 20%, 20 to 30%, 30 to 50%, 50 to 70%, 70 to 90%, 90 to 95%, or 95 to 99% by weight. In some embodiments, the concentrations of NMDP in pharmaceutical compositions range from 1 to 10%. In some embodiments, the concentrations of NMDP in pharmaceutical compositions range from 10 to 50%. In some embodiments, the concentrations of NMDP in pharmaceutical compositions range from 70 to 90%.

The concentration of API in pharmaceutical compositions can be at least about 0.1%, e.g., at least about 0.1%, 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, or 90% by weight. The concentration of API in pharmaceutical compositions can be less than about 99%, e.g., less than about 10%, 15%, 20%, 25%, 30%, 35%, 40%, 45%, 50%, 55%, 60%, 65%, 70%, 75%, 80%, 85%, 90% or 99% by weight. The concentration of API in pharmaceutical compositions can range from about 0.1 to 99%, e.g., 0.1 to 0.5%, 0.1 to 1%, 0.5 to 1%, 1 to 2%, 1 to 5%, 1 to 10%, 2 to 5%, 2 to 10%, 5 to 10%, 10 to 15%, 15 to 20%, 20 to 30%, 30 to 50%, 50 to 70%, 70 to 90%, 90 to 95%, or 95 to 99% by weight. In some embodiments, the concentrations of API in pharmaceutical compositions range from 1 to 10%. In some embodiments, the concentrations of API in pharmaceutical compositions range from 10 to 50%. In some embodiments, the concentrations of API in pharmaceutical compositions range from 70 to 90%.

The therapeutically effective amount of NMDP can be in a dry powder, semi-solid, a mucoadhesive formulation, intranasal vesicular unit or solution dosage form. The dry powder, semi-solid, a mucoadhesive formulation, intranasal vesicular unit or solution dosage form can have a unit weight of at least about 1 mg, e.g., at least about 1, 2, 3, 4, 5, 6, 7, 8, 9, 10, 20, 30, 40, 50, 60, 70, 80, 90, 100, 200, 300, 400, 500, 600, 700, 800, or 900 mg. The dry powder, semi-solid, a mucoadhesive formulation, intranasal vesicular unit or solution dosage form can have a unit weight of at least about 1 g, e.g., at least about 1, 2, 3, 4, 5, 6, 7, 8, 9, 10, 20, 30, 40, 50, 60, 70, 80, 90, or 100 g. The dry powder, semi-solid, a mucoadhesive formulation, intranasal vesicular unit or solution dosage form can have a unit weight of less than about 1000 mg, e.g., at least about 1, 2, 3, 4, 5, 6, 7, 8, 9, 10, 20, 30, 40, 50, 60, 70, 80, 90, 100, 200, 300, 400, 500, 600, 700, 800, or 900 mg. The dry powder, semi-solid, a mucoadhesive formulation, intranasal vesicular unit or solution dosage form can have a unit weight of less than about 100 g, e.g., less than about 1, 2, 3, 4, 5, 6, 7, 8, 9, 10, 20, 30, 40, 50, 60, 70, 80, 90, or 100 g. The dry powder, semi-solid, a mucoadhesive formulation, intranasal vesicular unit or solution dosage form can have a unit weight of ranges from about 1 mg to 10 g, e.g., ranges about 1 mg to 10 mg, 10 mg to 50 mg, 50 mg to 100 mg, 100 mg to 200 mg, 200 mg to 400 mg, 400 mg to 600 mg, 600 mg to 800 mg, 800 mg to 1 g, 1 g to 2 g, 2 g to 5 g, or 5 g to 10 g. The therapeutically effective amount of NMDP can be in a solution dosage form. The solution dosage form can have a unit volume of at least about 1 mL, e.g., at least about 1, 2, 3, 4, 5, 6, 7, 8, 9, 10, 20, 30, 40, 50, 60, 70, 80, 90, 100, 200, 300, 400, 500, 600, 700, 800, or 900 mL. The solution dosage form can have a unit volume of less than about 1000 mL, e.g., less than about 1, 2, 3, 4, 5, 6, 7, 8, 9, 10, 20, 30, 40, 50, 60, 70, 80, 90, 100, 200, 300, 400, 500, 600, 700, 800, or 900 mL. The solution dosage form can have a unit volume ranges from about 1 to 500 mL, e.g., ranges 1-500, 1-300, 1-100, 1-80, 1-60, 1-40, 1-20, 1-10, 1-5, 10-500, 10-300, 10-100, 10-80, 10-60, 10-40, 10-20, 20-500, 20-300, 20-100, 20-80, 20-60, 20-40, 40-500, 40-300, 40-100, 40-80, 40-60, 60-500, 60-300, 60-100, 60-80, 80-500, 80-300, 80-100, 100-500, 100-300, or 300-500 mL.

### Combination Treatment

The therapeutically effective amount of NMDP or a salt thereof can be the sole active pharmaceutical ingredients (API). Alternatively, the therapeutically effective amount of NMDP or a salt thereof can be used in combination with one or more additional API. Disclosed herein are pharmaceuticals, compositions, kits, and methods of preventing or treating a hearing disorder by administering NMDP or a salt thereof, e.g., therapeutically effective amount of NMDP or a salt thereof, and one or more active pharmaceutical ingredients (API), e.g., therapeutically effective amount of API.

The one or more API can comprise one or more sodium channel blockers, antioxidants, spin-trapping agents, N-methyl-D-aspartate (NMDA) antagonists, selective serotonin reuptake inhibitor (SSRI) antagonists, dopamine releasing agents (DRA), acetylcholine release inducers, norepinephrine reuptake inhibitors (NERI), monamineoxidase-A inhibitors (MAI), serotonin reuptake inhibitors (SRI), serotonin-norepinephrine reuptake inhibitors (SNRI), norepinephrine selective reuptake inhibitors (NSRI), serotonin reuptake inhibitors (5HT SRI), zonisamide, gabapentin, cannabinoid, or any combinations thereof.

Also disclosed herein are pharmaceuticals, compositions, kits, and methods for treating or preventing hearing disorders in a subject in need thereof comprising administering a therapeutically effective amount of NMDP or a salt thereof and one or more active pharmaceutical ingredients (API). The one or more API can be administered in therapeutically effective amount. The one or more API can comprise one or more sodium channel blockers, antioxidants, spin-trapping agents, N-methyl-D-aspartate (NMDA) antagonists, selective serotonin reuptake inhibitor (SSRI) antagonists, dopamine releasing agents (DRA), acetylcholine release inducers, norepinephrine reuptake inhibitors (NERI), monamineoxidase-A inhibitors (MAI), serotonin reuptake inhibitors (SRI), serotonin-norepinephrine reuptake inhibitors (SNRI), norepinephrine selective reuptake inhibitors (NSRI), serotonin reuptake inhibitors (5HT SRI), zonisamide, gabapentin, cannabinoid, or any combinations thereof.

The one or more API can comprise one or more antioxidants or spin-trapping agents. For example, the one or more antioxidants or spin-trapping agents can comprise allopurinol, glutathione, L-carnitine, methionine, or any combinations thereof. The one or more API can comprise one or more NMDA antagonists. For example, the one or more NMDA antagonists can comprise riluzole, caroverine, memantine, magnesium, or any combinations thereof. The one or more API can comprise one or more SSRI antagonists. For example, the one or more SSRI antagonists can comprise fluoxetine, sertraline, S-citalopram, alaproclate, or any combinations thereof. The one or more API can comprise one or more DRA. For example, the one or more DRA can comprise amantadine. The one or more API can comprise one or more acetylcholine release inducers or NERI. For example, the one or more acetylcholine release inducers or NERI can comprise bifemelane. The one or more API can comprise one or more MAI or SRI. For example, the one or more MAI or SRI can comprise pirlindole. The one or more API can comprise one or more SNRI. For example, the one or more SNRI can comprise milnacipran, bicifadine, or both. The one or more API can comprise one or more CCB. For example, the one or more CCB can comprise NMDP, verapamil, or both. The one or more API can comprise one or more NSRI. For example, the one or more NSRI can comprise atomoxetine. The one or more API can comprise one or more 5TH SRI. For example, the one or more 5TH SRI can comprise indeloxazine. The one or more API can comprise zonisamide.

The one or more API can comprise one or more drugs such as gabapentin. Other drugs that can be used are anticonvulsants. Other drugs can be used are drugs that stimulate gamma-aminobutyric acid (GABA) receptors. The one or more API can comprise a cannabinoid. For example, a cannabinoid can be marijuana or any extract of marijuana or synthetic composition that can stimulate the cannabinoid receptor, CB1 receptor, CB2 receptor, or a G-coupled receptor. These drugs can be used in in combination with nimodpine or a salt thereof.

### Kits

Disclosed herein are kits for treating or preventing hearing disorders in a subject in need thereof comprising NMDP or a salt thereof and a written instruction for treating or preventing hearing disorders using NMDP or a salt thereof. The NMDP or a salt thereof can be a therapeutically effective amount of NMDP or a salt thereof. The therapeutically effective amount of NMDP or a salt thereof can be in a tablet, capsule, caplet, spray, powder, gel cap, powder, or solution dosage form. For example, the therapeutically effective amount of NMDP or a salt thereof can be in a powder dosage form. The kit can further comprise a sterile solution. The kit can further comprise a sterile solution to be mixed with the powder dosage form prior to administration of the therapeutically effective amount of NMDP or a salt thereof. The kit can further comprise one or more active pharmaceutical ingredients (API). The one or more API can comprise one or more antioxidants, spin-trapping agents, N-methyl-D-aspartate (NMDA) antagonists, selective serotonin reuptake inhibitor (SSRI) antagonists, dopamine releasing agents (DRA), acetylcholine release inducers, norepinephrine reuptake inhibitors (NERI), monamineoxidase-A inhibitors (MAI), serotonin reuptake inhibitors (SRI), serotonin-norepinephrine reuptake inhibitors (SNRI), norepinephrine selective reuptake inhibitors (NSRI), serotonin reuptake inhibitors (5HT SRI), zonisamide, gabapentin, cannabinoid, or any combinations thereof.

### Molecular Mechanisms

Disclosed herein are methods for regulating calcium signaling pathways in a subject in need thereof comprising administering a therapeutically effective amount of NMDP or a salt thereof. The calcium signaling pathways can be regulated by inhibiting calcium influx via voltage-activated calcium channel, α2-adrenoceptor-operated calcium channel, or both. The calcium signaling pathways can be regulated by inhibiting cytosolic calcium pool release, for example, in corpus cavernosum smooth muscle cells. The calcium signaling pathways can be regulated by inhibiting tumor necrosis factor-alpha production, for example, induced by calcium entry. The calcium signaling pathways can be regulated by blocking the voltage-gated calcium channels. The calcium signaling pathways can be regulated by affecting the function of calcium-ATPase, calcium release channels, or both. The calcium signaling pathways can be regulated by releasing intracellular calcium, blocking calcium entry, or both. The calcium signaling pathways can be regulated by blocking endosomal calcium channels, e.g., two-pore channel. The calcium signaling pathways can be regulated by blocking T-type and/or L-type calcium current. The calcium signaling pathways can be regulated by inhibiting activity of BKCa channels. The calcium signaling pathways can be regulated by inhibiting calcium-release activated channels.

Disclosed herein are methods for regulating oxidative pathway in a subject in need thereof comprising administering a therapeutically effective amount of NMDP or a salt thereof. The oxidative pathway can be regulated by decreasing oxidative stress, down-regulating miRNA-155, decreasing TNF-α in the NF-κB signaling pathway, or a combination thereof. The oxidative pathway can be regulated by scavenging free radicals. The oxidative pathway can be regulated by lowering peroxide levels. The oxidative pathway can be regulated by regulating cellular redox states. The oxidative pathway can be regulated by inhibiting reactive oxygen species formation, suppressing up-regulation of Mac-1, neutrophil adhesion to fibrinogen, or any combination thereof. The oxidative pathway can be regulated by preventing hydrogen peroxide-induced oxidative neuronal cell damage.

Disclosed herein are methods for regulating anti-inflammation pathways in a subject in need thereof comprising administering a therapeutically effective amount of NMDP or a salt thereof. The anti-inflammation pathways can be regulated by reducing tube formation in the angiogenic process. For example, the reducing tube formation in the angiogenic process can inhibit on post-receptor pathway of IL-1α and/or platelet-derived growth factor-BB in chronic inflammation. The anti-inflammation pathways can be regulated by suppressing leukocyte infiltration into air pouches induced by IL-1 and/or TNF. The anti-inflammation pathways can be regulated by inhibiting prostaglandin E synthesis. The anti-inflammation pathways can be regulated by inhibiting IL-1, TNF-α IL-6, IL-8, IgG, phagocytosis by neutrophils, or any combination thereof. For example, the IL-1, TNF-α IL-6, and/or IL-8 can be from monocytes. The IgG can be from B cells. The anti-inflammation pathways can be regulated by suppressing lipopolysaccharide-induced increase of TNF-α, IL-1β, and/or high mobility group box 1 secretion by peritoneal macrophages.

### Patient Selection

In one aspect, presented herein a method of selecting a subject in need thereof for treatment of hearing disorder or a symptom of hearing disorder comprising: administering an L-type calcium channel blocker to the subject; selecting the patient for treatment of hearing disorder or a symptom of hearing disorder if the subject was responsive to the L-type calcium channel blocker; and administering a therapeutically effective amount of NMDP or a salt thereof to the subject if the subject was responsive to the L-type calcium channel blocker. In some embodiments, the L-type calcium channel blocker comprises carbamazepine. In some embodiments, the patient exhibits hearing loss. In some embodiments, the patient exhibits one or more symptoms related to a) hearing loss b) a change in auditory speech recognition as measured by a words-in-noise test, c) a change in auditory speech recognition as measured by a digits-in-noise test, d) a change in low-frequency hearing threshold e) a change in tinnitus or Meniere's disease severity g) a change in tinnitus or Meniere's disease loudness h) a change in vertigo severity i) a change in aural fullness j) a change in dizziness, and k) a change in hair cell function as observed when measured by a change in ABR threshold.

In one aspect, presented herein a method of selecting a subject in need thereof for treatment of tinnitus or Meniere's disease or a symptom of tinnitus or Meniere's disease comprising: administering an L-type calcium channel blocker to the subject; selecting the patient for treatment of tinnitus or Meniere's disease or a symptom of tinnitus or Meniere's disease if the subject was responsive to the L-type calcium channel blocker; and administering a therapeutically effective amount of NMDP or a salt thereof to the subject if the subject was responsive to the L-type calcium channel blocker. In some embodiments, the L-type calcium channel blocker comprises carbamazepine.

### Conditions to be Treated

The hearing disorders in the subject in need thereof can be NIHL, ARHL, hearing loss due to drugs or injury, or tinnitus or Meniere's disease. The hearing disorder in the subject can also include a combination of two or all four listed diseases. For example, tinnitus or Meniere's disease and the drug-induced hearing loss can be caused by an ototoxic drug. The ototoxic drug can comprise a chemotherapeutic agent, an antineoplastic agent, an antibiotic, a loop-diuretic, a quinine or quinine-like compound, or a salicylate or salicylate-like compound. In some embodiments, the ototoxic drug is not streptomycin. In some embodiments, the antibiotic is not streptomycin. In some embodiments, the hearing disorder is not caused by streptomycin.

Disclosed herein are pharmaceuticals, compositions, kits, and methods for the prevention and/or treatment of hearing disorders, including, but not limited to, various conditions such as noise-induced hearing loss (NIHL), age-related hearing loss (ARHL or presbycusis), drug or injury-induced hearing loss, central auditory hearing disorder (CAPD), tinnitus or Meniere's disease.

### NIHL—Noise-Induced Hearing Loss

NIHL is one of the most predominant health hazards posed by occupational and recreational settings. However, there are currently no FDA-approved drugs in diminishing NIHL, and the development of an efficacious treatment has been hampered by the complex array of cellular and molecular pathways involved in NIHL. NIHL can cause damages range from exhaustion of the hair cells in the ear to loss of those cells. Therefore, NIHL can be the consequence of overstimulation of the hair cells and supporting structures. Structural damage to hair cells (primarily the outer hair cells) can result in hearing loss that can be characterized by an attenuation and distortion of incoming auditory stimuli.

NIHL can be caused by a one-time exposure to excessive noise. For example, exposure to sound in excess of 80 dB, 90 dB, 100 dB, 110 dB, 120 dB, 130 dB, 140 dB, or 150 dB in short periods can cause NIHL. Alternatively, NIHL can also be caused by repeated exposure to noise over a period of time. For example, exposure to sound in excess of 60 dB, 65 dB, 70 dB, 75 dB, 80 dB, 85 dB, 90 dB, 95 dB, or 100 dB for more than 8 hours per day can cause NIHL. The symptoms of NIHL can include tinnitus or Meniere's disease, ear pain, hyperacusis, dizziness, vertigo and/or vestibular damages in the inner-ear.

Disclosed herein are pharmaceuticals, compositions, kits, and methods of treating or preventing hearing loss (e.g., NIHL) in a subject in need thereof wherein the methods comprise administering a therapeutically effective amount of NMDP or a salt thereof. In some embodiments, the therapeutically effective amount of NMDP or a salt thereof is an amount sufficient to reduce, or eliminate the NIHL. In some embodiments, the therapeutically effective amount of NMDP or a salt thereof is an amount sufficient to halt or prevent the NIHL. In some embodiments, the therapeutically effective amount of NMDP or a salt thereof is an amount sufficient to reverse the NIHL or at least partially restore hearing. The subject can be at risk of developing hearing loss (e.g., NIHL). Alternatively, the subject can be suffering hearing loss (e.g., NIHL).

### ARHL or Presbycusis

ARHL or presbycusis is a major health problem for which there are currently no treatments or preventatives. Age-related hearing loss develops gradually over time and in its early stages can be practically imperceptible to the affected individual. The cause of an ARHL or presbycusis is generally considered to be degeneration of the auditory nervous system, especially the auditory nerves in the ears. It is the most common form of hearing loss in persons over 55 years of age.

Early noise injury can be a cause of ARHL or presbycusis. The subject can be suffering or at risk of developing hearing loss (e.g., ARHL or presbycusis). For example, the subject can be suffering or at risk of developing hearing loss (e.g., ARHL or presbycusis) at least at 30 years, 35 years, 40 years, 45 years, 50 years, 55 years, 60 years, 65 years, 70 years, 75 years, 80 years, 85 years, 90 years, 95 years, or 100 years of age. In some embodiments, the subject can be 50 years of age.

Disclosed herein are pharmaceuticals, compositions, kits, and methods of treating or preventing hearing loss (e.g., ARHL or presbycusis) in a subject in need thereof wherein the methods comprise administering a therapeutically effective amount of NMDP or a salt thereof. In some embodiments, the therapeutically effective amount of NMDP or a salt thereof is an amount sufficient to reduce, or eliminate the ARHL or presbycusis. In some embodiments, the therapeutically effective amount of NMDP or a salt thereof is an amount sufficient to halt or prevent the ARHL or presbycusis. In some embodiments, the therapeutically effective amount of NMDP or a salt thereof is an amount sufficient to reverse the ARHL or presbycusis or at least partially restore hearing. The subject can be at risk of developing hearing loss (e.g., ARHL or presbycusis). Alternatively, the subject can be suffering hearing loss (e.g., ARHL or presbycusis).

### Traumatic Brain Injury and Subarachnoid Hemorrhage

More than half of all traumatic brain injury (TBI) patients develop tinnitus, and greater than 20% of patients who experience a subarachnoid hemorrhage (SAH) develop hearing impairments. Accordingly, in some aspects, the compositions and methods disclosed herein can be administered for the treatment of TBI and/or SAH induced hearing impairments. In some embodiments, the NMDP formulations disclosed herein can be administered for the treatment of the TBI and/or SAH induced hearing impairments such as tinnitus.

### Drug-Induced Hearing Loss

Ototoxic drugs, such as chemotherapeutic agents, antineoplastic agents, antibiotics, loop-diuretics, quinines or a quinine-like compounds, and salicylate or salicylate-like compounds, can cause drug-induced hearing loss. For example, aminoglycosides are antibiotics that have been used for the treatment of Gram-negative bacterial infections and some aerobic Gram-positive bacterial infections. Despite their utility, however, they have serious side effects, including ototoxicity associated with the destruction of the sensory hair cells in organ of Corti of the cochlea of the inner ear. In addition, surgery near or on auditory nerves can cause hearing loss, which subsequently causes tinnitus or tinnitus like symptoms.

Disclosed herein are pharmaceuticals, compositions, kits, and methods of treating or preventing hearing loss (e.g., injury or drug-induced hearing loss) in a subject in need thereof wherein the methods comprise administering a therapeutically effective amount of NMDP or a salt thereof. In some embodiments, the therapeutically effective amount of NMDP or a salt thereof is an amount sufficient to reduce, or eliminate this type of hearing loss. In some embodiments, the therapeutically effective amount of NMDP or a salt thereof is an amount sufficient to halt or prevent this type of hearing loss. In some embodiments, the therapeutically effective amount of NMDP or a salt thereof is an amount sufficient to not only prevent the drug-induced hearing loss or at least partially restore hearing. The subject can be at risk of developing hearing loss (e.g., drug-induced hearing loss). Alternatively, the subject can be suffering hearing loss (e.g., drug-induced hearing loss). In some embodiments, the therapeutically effective amount of NMDP or a salt thereof is an amount sufficient to not only prevent the drug-induced hearing loss, but works synergistically with cancer drugs to kill cancer cells

The therapeutically effective amount of NMDP or a salt thereof can prevent onset of an injury- or drug-induced hearing loss. For example, disclosed herein are methods for preventing a drug-induced hearing loss, comprising administering a therapeutically effective amount of NMDP or a salt thereof to a subject in need thereof prior to administering to the subject one or more ototoxic drugs for the treatment of a condition other than hearing loss. The administering a therapeutically effective amount of NMDP or a salt thereof can begin up to about 12 months (e.g., 1 day to 60 days) prior to the administration of the one or more ototoxic drugs. The administering a therapeutically effective amount of NMDP or a salt thereof can also begin on the same day as the administration of the one or more ototoxic drugs. In some embodiments, the administering a therapeutically effective amount of NMDP or a salt thereof can begin up to about 12 months, e.g., 1 hour, 6 hours, 12 hours, 24 hours, 2 days, 4 days, 6 days, 8 days, 10 days, 20 days, 1 month, 2 months, 4 months, 6 months, 8 months, 10 months, or 12 months prior to the administration of the one or more ototoxic drugs. In some embodiments, the administering a therapeutically effective amount of NMDP or a salt thereof can begin 1 day to 12 months, e.g., 1 day to 2 days, 2 days to 10 days, 10 days to 1 months, 1 months to 3 months, 3 months to 6 months, or 6 months to 12 months, prior to the administration of the one or more ototoxic drugs.

The therapeutically effective amount of NMDP or a salt thereof can treat or prevent drug-induced hearing loss, such as amelioration of drug-induced hearing loss, reduction or elimination of tinnitus or Meniere's disease, partial or total rehabilitation of hearing, or prevention of further hearing loss arising out of ototoxic effects of the one or more ototoxic drugs. The methods disclosed herein provide for dosing of a pharmaceutical composition in response to a noted decrease in hearing function arising out of, or occurring during, dosing of one or more ototoxic drugs.

The administering a therapeutically effective amount of NMDP or a salt thereof can be continued for the duration of the one or more ototoxic drugs. The administering a therapeutically effective amount of NMDP or a salt thereof can stop on the same day as the cessation of the one or more ototoxic drugs. The administering a therapeutically effective amount of NMDP or a salt thereof can continue for at least about 1 day, e.g., for at least about 1 day, 2 days, 4 days, 6 days, 8 days, 10 days, 20 days, 1 month, 2 months, 4 months, 6 months, 8 months, 10 months, or 12 months after cessation of the one or more ototoxic drugs. In some embodiments, the administering a therapeutically effective amount of NMDP or a salt thereof can continue for at least about 1 day to 12 months, e.g., 1 day to 2 days, 2 days to 10 days, 10 days to 1 months, 1 months to 3 months, 3 months to 6 months, or 6 months to 12 months after cessation of the one or more ototoxic drugs.

Some examples of ototoxic drugs include certain antibacterial and antineoplastic drugs. For example, some ototoxic drugs are chemotherapeutic agents, e.g., antineoplastic agents, and antibiotics. Other possible candidates include loop-diuretics, quinines or a quinine-like compound, and salicylate or salicylate-like compounds. Thus, disclosed herein are pharmaceuticals, compositions, kits, and methods of treating or preventing hearing loss caused by an ototoxic drug, wherein the ototoxic drug can be an antineoplastic agent (e.g., ototoxic aminoglycoside antibiotic) such as cisplatin, an antibiotic such as an aminoglycoside, a loop-diuretic, a quinine, a quinine-like compound, a salicylate or salicylate-like compound.

Ototoxic aminoglycoside antibiotics include but are not limited to neomycin, paromomycin, ribostamycin, lividomycin, kanamycin, amikacin, tobramycin, viomycin, gentamicin, sisomicin, netilmicin, streptomycin, dibekacin, fortimicin, and dihydrostreptomycin, or combinations thereof. Particular antibiotics include neomycin B, kanamycin A, kanamycin B, gentamicin C1, gentamicin C1a, and gentamicin C2. Thus, disclosed herein are pharmaceuticals, compositions, kits, and methods of treating or preventing drug-induced hearing loss comprising administering to a subject, who has been, is being or will be treated with one or more aminoglycosides, a therapeutically effective amount of a pharmaceutical composition of the disclosure. In some embodiments, the ototoxic aminoglycoside antibiotic is not streptomycin. In some embodiments, the hearing disorder is not caused by ototoxic aminoglycoside antibiotics. In some embodiments, the hearing disorder is not caused by streptomycin.

Hearing impairments induced by aminoglycosides can be prevented or reduced by the pharmaceuticals, compositions, kits, and methods disclosed herein. Although the aminoglycosides are particularly useful due to their rapid bactericidal action against infections of aminoglycoside-susceptible organisms, their use has heretofore been limited to more severe, complicated infections because of ototoxic and nephrotoxic side-effects. For this reason the aminoglycosides have been considered to have a low therapeutic/risk ratio compared to other antibiotics used systemically. Thus, disclosed herein are also improved methods of treatment of aminoglycoside-susceptible infections, comprising administering to a subject an anti-bacterially effective amount of an aminoglycoside and a pharmaceutical composition disclosed herein. It is to be recognized that recommended doses of aminoglycosides have been established; and the methods disclosed herein are effective when administering aminoglycosides in a range of about 100 to about 500%, in particular about 100 to about 250%, and more particularly about 100 to about 150% of the currently recommended doses, which are available in general in the product labeling and package inserts for the commercially available drug aminoglycoside drug products. The improved methods provide prophylaxis against aminoglycoside-induced hearing loss and/or tinnitus or Meniere's disease, thereby expanding the therapeutic index of the aminoglycoside drug.

The disclosed pharmaceutical compositions can be co-administered with one or more ototoxic drugs in the same dosage form. For example, an improved method is provided for treatment of infection of a subject by administration of an aminoglycoside antibiotic and a therapeutically effective amount of a pharmaceutical composition disclosed herein. Alternatively, the aminoglycoside antibiotic and the pharmaceutical composition disclosed herein can be administered to the subject in separate dosage forms.

The one or more ototoxic drugs can also be chemotherapeutic drugs for treatment of cancer in a subject. For example, an improved method is provided for treatment of cancer in a subject by administration of a chemotherapeutic drug (e.g., antineoplastic chemotherapeutic agent) and a therapeutically effective amount of a pharmaceutical composition disclosed herein.

Ototoxic antineoplastic chemotherapeutic agents include cisplatin or cisplatin-like compounds, taxol or taxol-like compounds, and other chemotherapeutic agents believed to cause ototoxin-induced hearing impairments, e.g., vincristine, an antineoplastic drug used to treat hematological malignancies and sarcomas. Thus, the methods disclosed herein can be used to treat ototoxicity (e.g., drug-induced hearing loss) in a subject, who will be, is being, or has been treated with an antineoplastic agent, including cisplatin or cisplatin-like compounds, taxol or taxol-like compounds, and other chemotherapeutic agents believed to cause ototoxin-induced hearing impairments, e.g., vincristine, an antineoplastic drug used to treat hematological malignancies and sarcomas.

### Central Auditory Hearing Disorder (CAPD)

Central auditory processing disorders (CAPD) relate to difficulties in the perceptual processing of auditory information in the central nervous system (CNS). Tests for CAPD can include: auditory discrimination tests; auditory temporal processing and patterning tests; dichotic speech tests; monaural low-redundancy speech tests; binaural interaction tests; electroacoustic measures; and electrophysiological measures.

Disclosed herein are pharmaceuticals, compositions, kits, and methods of treating or preventing hearing loss (e.g., CAPD) in a subject in need thereof wherein the methods comprise administering a therapeutically effective amount of NMDP or a salt thereof. In some embodiments, the therapeutically effective amount of NMDP or a salt thereof is an amount sufficient to reduce, or eliminate the CAPD. In some embodiments, the therapeutically effective amount of NMDP or a salt thereof is an amount sufficient to halt or prevent the CAPD. In some embodiments, the therapeutically effective amount of NMDP or a salt thereof is an amount sufficient to reverse the CAPD or at least partially restore hearing. The subject can be at risk of developing hearing loss (e.g., CAPD). Alternatively, the subject can be suffering hearing loss (e.g., CAPD).

### Tinnitus

Tinnitus is the perception of sound in the ears even without external auditory stimulation. The most frequent manifestation of tinnitus is a ringing in the ears; however, tinnitus can also present as crickets, whooshing, pulsing, ocean waves, buzzing, even music, Tinnitus can be temporary, intermittent or even permanent; and its severity can range from a quiet background ringing to an overwhelming auditory sensation that drowns out external sources of sound

Tinnitus can be caused by one or more factors, such as administration of, or exposure to, ototoxic substances (such as an aspirin overdose), exposure to a short burst of extreme noise (e.g., gunshot or explosion) or prolonged exposure to high decibel noise (such as aircraft engine noise, high decibel music concerts or high decibel headphone usage), or central auditory processing disorders as discussed herein.

Disclosed herein are pharmaceuticals, compositions, kits, and methods of treating or preventing hearing disorder (e.g., tinnitus) in a subject in need thereof wherein the methods comprise administering a therapeutically effective amount of NMDP or a salt thereof. In some embodiments, the therapeutically effective amount of NMDP or a salt thereof is an amount sufficient to reduce, or eliminate the hearing disorder (e.g., tinnitus). In some embodiments, the therapeutically effective amount of NMDP or a salt thereof is an amount sufficient to halt or prevent the hearing disorder (e.g., tinnitus), or treat a symptom thereof. In some embodiments, the therapeutically effective amount of NMDP or a salt thereof is an amount sufficient to reverse the hearing disorder (e.g., tinnitus) or at least partially restore hearing. The subject can be at risk of developing the hearing disorder (e.g., tinnitus). Alternatively, the subject can be suffering the hearing disorder (e.g., tinnitus).

### Meniere's Disease

Meniere's disease is a disorder of the inner ear that can lead to ringing in the ears (tinnitus), reduction or loss in hearing, vertigo and a feeling of fullness or congestion in the ear. Attacks of dizziness can come on suddenly or after a short period of tinnitus or muffled hearing. Some people will have single attacks of dizziness separated by long periods of time. Others can experience many attacks closer together over a number of days. Some people with Meniere's disease have vertigo so extreme that they lose their balance and fall. Meniere's disease is often a severe and debilitating condition for afflicted patients.

Meniere's disease can develop at any age, but it is more likely to happen to adults between 40 and 60 years of age. The National Institute on Deafness and Other Communication Disorders (NIDCD) estimates that there are approximately 615,000 individuals in the United States, currently diagnosed with Meniere's disease, with 45,500 cases newly diagnosed each year. Meniere's disease usually affects only one ear. Without being bound to a particular theory, the symptoms of Meniere's disease can be caused by the buildup of fluid in the compartments of the inner ear. There is no known cure for Meniere's disease. There remains a significant need for improved pharmaceutical compositions and methods of use for treatment of Meniere's disease.

### Salts, Stereoisomers, Polymorphs and Derivatives

Although described above with reference specific to compounds, one can also utilize stereoisomers, polymorphs, metabolites, derivates, and/or salts of the active compounds. Examples of therapeutically acceptable salts include, but are not limited to, mineral or organic acid salts of basic residues such as amines, and alkali or organic salts of acidic residues such as carboxylic acids. The therapeutically acceptable salts include the conventional non-toxic salts or the quaternary ammonium salts of the parent compound formed, for example, from non-toxic inorganic or organic acids. Conventional non-toxic salts include those derived from inorganic acids such as hydrochloric, hydrobromic, sulfuric, sulfamic, phosphoric and nitric acid; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic and isethionic acids. The therapeutically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; example, non-aqueous media like ether, ethyl acetate, ethanol, isopropanol, or acetonitrile. Lists of suitable salts are found in Remington's Pharmaceutical Sciences, 17th ed. (Mack Publishing Company, Easton, Pa, 1985, p. 1418).

Stereoisomers are compounds made up of the same atoms having the same bond order but having different three-dimensional arrangements of atoms which are not interchangeable. The three-dimensional structures are called configurations. Two kinds of stereoisomers include enantiomers and diastereomers. Enantiomers are two stereoisomers which are non-superimposable mirror images of one another. This property of enantiomers is known as chirality. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g., Jacques, J. et al., “Enantiomers, Racemates, and Resolutions”, John Wiley and Sons, Inc. 1981). Diastereomers are two stereoisomers which are not mirror images but also not superimposable. Diastereoisomers have different physical properties and can be separated from one another easily by taking advantage of these differences.

A metabolite of the above-mentioned compounds results from biochemical processes by which living cells interact with the active parent drug or other formulas or compounds in vivo. Metabolites include products or intermediates from any metabolic pathway.

### API

NMDP is a calcium channel blocker (CCB) belonging to the dihydropyridine class and is a highly lipophilic agent that rapidly crosses the blood-brain barrier. The chemical structure and associated properties are shown below. NMDP can be used to treat tinnitus associated with hearing loss, vertigo and tinnitus associated with Meniere's disease, traumatic brain injury and subarachnoid hemorrhage and to prevent hearing loss during surgery for vestibular Schwannomas.

NMDP belongs to class II of the Biopharmaceutical Classification System (BCS). It exhibits poor water solubility, but good permeability characteristic due to its high lipophilicity (log p=3.41). NMDP is a weak basic compound with predicted pka value equals to 5.41. NMDP is freely soluble in ethanol. NMDP is available in several FDA approved dosage forms, soft gelatin capsules, tablets, liquid for intravenous administration, and oral solution (Sweetman and Martindale, 2002). Some of the physical properties of NMDP (NMD) are shown in the below table.

| Property | NMDP |
|---|---|
| MW | 418.4 |
| Water solubility | 0.012 mg/mL |
| Log P | 3.05 |
| M.P. | 125° C. |
| Hydrogen Bond Donor | 1 |
| Count | |
| Hydrogen Bond Acceptor | 8 |
| Count | |
| pKa | 5.41 |

In humans, NMDP can be rapidly absorbed after oral administration, and the peak concentrations can be attained within one hour. Bioavailability outside of the central nervous system can be 100% following intravenous administration and 3-30% following oral administration due to extensive first-pass metabolism. Only 5% of NMDP reach to the brain due to high affinity binding of plasma proteins with NMDP.

### API Concentrations

The composition of the disclosure typically has a concentration equivalent to about 0.4 to 75 mg/mL of NMDP, 0.5 to 40 mg/mL of NMDP, 0.6 to 35 mg/mL, 0.7 to 25 mg/mL, 0.75 to 15 mg/mL of NMDP, or 1 to 10 mg/mL NMDP. Suitable compositions have a concentration equivalent to about at least 0.4 mg/mL of NMDP, 1 mg/mL, 2 mg/mL, 3 mg/mL, 4 mg/mL or 5 mg/mL of NMDP, such as 7 mg/mL, such as 10 mg/mL, such as about 15 mg/mL, about 20, about 25, about 35, about 45, about 60, about 80, about 100, about 150, about 125, about 150, about 180, about 225, about 275, and about 400 mg/mL NMDP.

As stated, the composition is delivered as a dosage unit wherein administration comprises delivery of one or more dosage units of about 10 to 500 μL, such as 10 to 200 μL, preferably about 50 to 150 μL. In the embodiment wherein delivery is via the nasal mucosa, a delivery unit corresponds to the volume provided by a squirt or spray, depending on the device utilized for delivery of the composition and dosage unit.

In the event wherein the nasal application exceeds about 200 μl, there can be a risk of loss of the formulation to the larynx or loss through the nostrils. Accordingly, the formulation for nasal administration cannot exceed 200 μl per application in some embodiments. Accordingly, a volume according to the disclosure includes a volume selected from 10 μl, 25 μl, 50 μl, 75 μl, 100 μl, 150 μl, 200 μl, 250 μl, 300 μl, and 350 μl, and 400 μl where the volume can be delivered to both nostrils if preferred.

In one embodiment, said composition is formulated for nasal delivery of a dosage unit comprising an equivalent to at least about 70 μg of NMDP, such as 80, 90, or 100 μg, such as 125, 150, 200, 250, or 300 μg, such as 350, 400, 450, 500 μg, such as 550, 600, 650, 700, 750, 800, 850, 900, or 950 μg, such as 1000, 1050, 1100, 1250, or 1300 μg, such as 1350, 1400, 1450, 1500 μg, such as 1550, 1600, 1650, 1700, 1750, 1800, 1850, 1900, or 1950 μg, such as a dosage unit equivalent to 2000 μg of NMDP.

Alternatively, defined, the composition is formulated for transmucosal delivery of a dosage unit equivalent to about 70 to 2500 mg of NMDP, for example 70 to 1800 mg, 70 to 1500 mg, 70 to 1200 mg, 70 to 1000 mg, 70 to 500 mg, to 75 to 300 mg of NMDP.

The present disclosure further relates to a method for the administration of NMDP or a pharmaceutically acceptable salt thereof to the circulatory system of an individual in need of acute tinnitus or Meniere's disease or a symptom of tinnitus or Meniere's disease relief. The treatment dosage is such that to be sufficient to treat the acute tinnitus or Meniere's disease or a symptom of tinnitus or Meniere's disease within a narrow time-to-onset-of-action. In order to result in a plasma concentration sufficient to treat acute tinnitus or Meniere's disease or a symptom of tinnitus or Meniere's disease, a treatment dosage will normally be within a range of at least about 1 to about 5 mg/kg or about 50 mg to about 250 mg. In order to have the NMDP administered to the circulatory system within an acceptable period and without delivering the NMDP by injection, the NMDP is administered to a mucosal membrane of the patient in a pharmaceutical vehicle for transmucosal delivery of the NMDP.

The sufficient tinnitus or Meniere's disease or a symptom of tinnitus or Meniere's disease relieving dosage can vary between the patients as well as in the individual patient. For treatment of relative moderate acute tinnitus or Meniere's disease or a symptom of tinnitus or Meniere's disease, the treatment dosage can comprise at least 70 μg NMDP, preferably at least 100 μg NMDP, at least 150 μg NMDP, such as 200 μg NMDP. For treatment of more severe acute tinnitus or Meniere's disease or a symptom of tinnitus or Meniere's disease, the treatment dosage comprises at least 250 μg NMDP, preferably at least 300 μg NMDP, at least 400 μg NMDP, such as 500 μg NMDP, 1000 μg NMDP, 1500 μg NMDP, 2000 μg NMDP. In cases where the patient suffers from heavy acute tinnitus or Meniere's disease or a symptom of tinnitus or Meniere's disease or the patient has developed tolerance to NMDP higher dosages can be required and administered according to the present disclosure. Such high dosages include treatment dosage comprising 25 mg NMDP, at least 30 mg NMDP, such as at least 40 mg NMDP, at least 50 mg NMDP, at least 100 mg, at least 150 mg, at least 200, at least 400 mg, at least 600 mg, at least 1000 mg. Even higher dosages can be desired such as treatment dosages of 1300 mg NMDP, preferably at least 1400 mg NMDP, at least 1500 mg NMDP, such as 1700 mg NMDP. In some embodiments, the treatment can involve patients that can need treatment dosage comprising from 1800 to 2500 mg NMDP.

As stated, the compositions of the disclosure for transmucosal delivery are of a more potent concentration than compositions known to the person skilled in the art. In one embodiment, the composition is formulated such that a treatment dosage comprises about 1, 2, 3, 4, 5, 6, 7, 8, 9, 10, 11, 12, 13, 14, 15, 16, 17, 18, 19, 20, 21, 22 25, 30, 35, 40, 45, 50, 100, or 300 dosage units.

It is an important aspect of the disclosure that the tinnitus or Meniere's disease or a symptom of tinnitus or Meniere's disease relief can be obtained shortly upon administration of NMDP. Accordingly, the relief of the acute tinnitus or Meniere's disease or a symptom of tinnitus or Meniere's disease should be obtained shortly after administration of the first delivery of the dosage unit or treatment dosage such that the administration of composition has a time-to-onset-of-action of less than 10 minutes, such as less than 9 minutes, or less than 8 minutes. In some embodiments, the composition can have a time-to-onset-of-action of greater than 10 minutes, for example 15 minutes, 20 minutes, 25 minutes, 30 minutes, or greater than 30 minutes.

In some embodiments, in addition to the very short a time-to-onset-of-action, that the tinnitus or Meniere's disease or a symptom of tinnitus or Meniere's disease relief is maintained for at least 30 minutes. In some embodiments, administration of the composition has a duration-of-action maintained throughout a period of at least 30 minutes, at least 60 minutes, at least 90 minutes, at least 120 minutes, at least 240 minutes, at least 360 minutes, at least 480 minutes, at least 600 minutes, at least 720 minutes, at least 840 minutes, at least 960 minutes, or at least 1080 minutes, 20 hours, 22 hours, 24 hours, 36 hours, 48 hours, or 60 hours.

The composition has a pseudo “sustained release” effect in comparison to intravenous administration which has a very rapid onset of action but with a very short duration of action. The intranasal composition described herein, upon administration, typically has a bioavailability of no less than 75% that of intravenous administration, for example no less than 80% of intravenous administration, or no less than 90% of intravenous administration. Bioavailability can be determined by its AUC, as is known to the person skilled in the art.

The method of the disclosure comprises the administration of dosage units comprising from about 70 to 2500 mg of NMDP, said administration resulting in a C_{max,nasal}/C_{max,iv} ratio which decreases with increasing dosage units delivered of equal amounts of NMDP, within the treatment dosage range of from about 70 to 2500 mg.

### API Formulations

To facilitate intranasal administration of NMD, an effective amount of NMDP should be dissolved or finely dispersed in a small volume of liquid vehicle, for example, less than about 1,000 μL (microliter), preferably less than 300 μL, and for example less than 150 μL. Larger Volumes drain out anteriorly through the nostrils or posteriorly toward the pharynx where excess liquid is swallowed. As a result, if large volumes are administered, a portion of NMDP can be lost from the absorption site, and it can be difficult if not impossible to reproducibly administer the correct dose of the therapeutic agent. Thus, it is desirable to have a high dissolved NMDP concentration or NMDP content dispersed in a small volume of liquid vehicle for intranasal administration. Furthermore, to support drug absorption through the nasal mucosa, two natural protective functions must be bypassed—the mucociliary clearance (MCC) and the barrier properties of the tissue. It is desirable that an effective dosage can bypass natural attributes of the mucus blanket as a protective layer, i.e., increase drug absorption, and of the MCC as an effective cleansing mechanism. In other words, it is desirable to increase resident time of the applied dose on the mucous layer. The surfactants suitable for this invention are non-ionic surfactants. The non-ionic surfactants suitable for this invention are polysorbates, particularly polysorbate 20 (Tween 20) and polysorbate 80 (Tween 80). Tween-80 (polyoxyethylene sorbitan monooleate) is oleate and Tween-20 (polyoxyethylene sorbitan monolaurate) is laurate. Both are listed in the FDA inactive ingredient list for nasal spray applications. Both Tween 20 and Tween 80 are miscible with water. The chemical structure of Tween 80 is shown below.

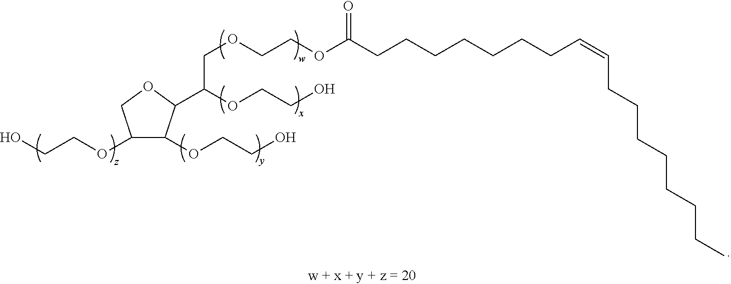

The water-soluble cellulosic polymers suitable for use in this invention include, but not limited to, hydroxypropyl methylcellulose (HPMC; Hypromellose as another name), hydroxyethyl cellulose (HEC), carboxymethylcellulose (CMC), hydroxypropyl cellulose (HPC), sodium CMC, sodium CMC and MCC, xanthan gum, guar gum, acacia gum, natural gums such as tragacanth gum and/or carboxymethylcellulose sodium. These polymers are also listed in the FDA inactive ingredient list for nasal spray applications. HPMC is the preferred polymer to be used in this invention. HPMC is a nonionic water-soluble cellulose derivative with hydrogen bonding potential and stabilizing ability to prevent crystallization of amorphous material. HPMC, ~1% in phosphate buffer solution (pH 6.8), can also increase the drug solubility. In some embodiments the HPMC used for the formulation exhibits the viscosity values for 2% (w/v) aqueous solutions at 20 C between about 2-4,000 mPa·s, about 4-60 mPa·s, and/or, about 4-6 mPa·s. In some instances, the HPMC is E6, E50 or E4M. In some embodiments, the preferred HPMC used for the formulation can comprise a molecular weight of about 50 to about 100 kDa, about 50 to about 150 kDa, about 50 to about 200 kDa, about 50 to about 250 kDa, about 50 to about 300 kDa, about 50 to about 350 kDa, about 50 to about 400 kDa, about 50 to about 500 kDa, about 50 to about 600 kDa, about 50 to about 700 kDa about 50 to about 800 kDa, about 50 to about 900 kDa, about 50 to about 1100 kDa, about 50 to about 1500 kda, about 50 to about 100 kDa, about 100 to about 150 kDa, about 100 to about 200 kDa, about 100 to about 250 kDa, about 100 to about 300 kDa, about 100 to about 350 kDa, about 100 to about 400 kDa, about 100 to about 500 kDa, about 100 to about 600 kDa, about 100 to about 700 kDa about 100 to about 800 kDa, about 100 to about 900 kDa, about 100 to about 1100 kDa, or about 100 to about 1500 kda. In some embodiments, Polyethylene glycol (PEG) or it methoxy derivative like methoxy-polyethylene glycol (mPEG), or a combination of mPEG/PEG, can be good solvents for poorly water-soluble NMDP. In some embodiments, low molecular weight PEG, mPEG, and/or a mixture of PEG and mPEG can be used. In some embodiments, PEG, mPEG, and/or a mixture of PEG and mPEG can be used to prepare a low-viscosity formulation.

The structure of polyethylene glycol (PEG) is represented in I below:

H—O—(CH2CH2O)—H I:

wherein n is a number in the range of 1 to 25.

The structure of an alkoxy-polyethylene glycol is represented by II below:

R—O—(CH2CH2O)—H II:

wherein, R is methyl, ethyl, n-propyl, isopropyl, or cyclopropyl; and n, which is the average number of oxyethylene repeating units, is a number in the range of from about 1 to about 25.

### Measurement of the Effect of the Pharmaceutical Compositions

The composition is intended for the treatment, alleviation or lessening of acute or breakthrough tinnitus or Meniere's disease or a symptom of tinnitus or Meniere's disease, based on an assessment that comprises a) measurement of a change in ABR threshold, b) a change in auditory speech recognition as measured by a words-in-noise test, c) a change in auditory speech recognition as measured by a digits-in-noise test, d) a change in low-frequency hearing threshold e) a change in incidence of adverse events after administration of the pharmaceutical composition, f) a change in tinnitus or Meniere's disease severity g) a change in tinnitus or Meniere's disease loudness h) a change in vertigo severity i) a change in aural fullness j) a change in dizziness, and k) a change in hair cell function as observed when measured by a change in ABR threshold, after administration of the pharmaceutical composition.

In a further aspect, the composition, dosage unit, use and method of the disclosure is characterized by the effect of the treatment on the acute tinnitus or Meniere's disease or a symptom of tinnitus or Meniere's disease as measured as described herein. One way to record tinnitus or Meniere's disease or a symptom of tinnitus or Meniere's disease according to the disclosure comprises the measurement of onset of tinnitus or Meniere's disease or a symptom of tinnitus or Meniere's disease relief. Just before administration of the treatment the time is measured, e.g., by starting a stopwatch. When the subject is certain of feeling a meaningful tinnitus or Meniere's disease or a symptom of tinnitus or Meniere's disease relief the time is recorded, e.g., by stopping the stopwatch. The composition of the disclosure, upon administration, has a tinnitus or Meniere's disease or a symptom of tinnitus or Meniere's disease reduction score in the range of 2 to 7, such as 2, 3, 4, 5, 6, and 7, preferably such as 3, 4, 5, and 6, as measured by PID upon delivery of no more than two dosage units, preferably after delivery of one dosage unit.

At least 50% of subjects obtaining onset within 15 minutes after administration of treatment will be considered a success. Likewise the duration of effect can be measured as the difference between onset of effect and the time point where the subject declares the effect to cease or the time when the subject takes rescue medication, whatever comes first. Duration of tinnitus or Meniere's disease or a symptom of tinnitus or Meniere's disease relief of at least half an hour experienced by at least 50% of the subjects will be considered a success.

Another measurement is the Tinnitus or Meniere's disease or a symptom of tinnitus Intensity (TI) scored on an 11-point numeric rating scale (0=no tinnitus or a symptom of tinnitus, 10-unendurable tinnitus or a symptom of tinnitus). TIᵢ is the tinnitus or a symptom of tinnitus intensity at the time point Tᵢ. The TIᵢ is measured at one or more of the following time points (Tᵢ) before treatment (baseline), at the time of meaningful tinnitus or a symptom of tinnitus relief, every 15 minutes after administration of treatment for the two first hours, and every 30 minutes for the next two hours. A 40% decrease of mean TI within 15 minutes after treatment can be considered a success. Naturally other time points can and intervals can be selected.

TI₀ is the baseline tinnitus or a symptom of tinnitus intensity (scored on a scale as disclosed above) before administration of treatment (at time T₀). Tinnitus or a symptom of tinnitus Intensity Difference (TID) is the TI₀ compared to the tinnitus or a symptom of tinnitus intensity at time points after the administration of treatment (TIᵢ). A mean TID of 2 obtained within 15 minutes after administration will be considered a success.

A further measurement is the area under the TID curve or the Sum of Tinnitus or a symptom of tinnitus Intensity Difference (STID), TI being measured at the time points disclosed above. A mean 4-hour STID of 3 will be considered a success.

One method relates to a tinnitus or Meniere's disease or a symptom of tinnitus intensity scale as disclosed herein wherein tinnitus or a symptom of tinnitus relief is measured as a tinnitus or a symptom of tinnitus intensity difference (TID) of at least 30%, such as at least 40% based on a tinnitus or a symptom of tinnitus score measured close to the time of the administration TI₀ and a tinnitus or a symptom of tinnitus score measured at the time TIᵢ after administration. The time after administration can be selected from the time of one or more of the following times 3 minutes, 5 minutes, 7 minutes, 10 minutes, 15 minutes, 20 minutes, and 30 minutes upon administration. These times is used when the purpose of the measuring is to evaluate the immediate effect of the administration. If a measurement of the duration of treatment is desired, the tinnitus or Meniere's disease or a symptom of tinnitus or Meniere's disease relief is measured as a tinnitus or Meniere's disease or a symptom of tinnitus or Meniere's disease intensity difference (TID) based on a score measured immediately before the administration TI₀ and at the time TIᵢ after administration, the time after administration being selected from the time 45 minutes, 60 minutes, 75 minutes, 90 minutes, and 120 minutes upon administration. One alternative is to measure an effect from a given time after administration to a later time, and in this respect the desired time range is selected individually.

The tinnitus or Meniere's disease or a symptom of tinnitus or Meniere's disease relief score can be measured in accordance with the method disclosed herein or on a scale of 1-100% wherein 100% is a tinnitus or a symptom of tinnitus described by the patient as unbearable and 0% is no tinnitus or a symptom of tinnitus at all. It is preferred that the score is at least 30% from the start to the maximum relief effect is obtained.

A further measurement is as explained above the sum of tinnitus or a symptom of tinnitus intensity difference (STID) based on a score measured immediately before the administration PI₀ and at the time PIᵢ after administration, the time after administration being selected from the time any time as desired and includes the times as disclosed herein. In an embodiment, the sum of tinnitus or a symptom of tinnitus intensity difference is measured from at least 2 values measured during a period of at least 30 minutes, preferably at least during 45 minutes, preferable at least during 60 minutes such as during 90 minutes. Furthermore, the sum of tinnitus or a symptom of tinnitus intensity difference can be measured from at least 5 values such as at least from 7 values, preferable from at least 10 values such as from 11, 12 or 13 values.

In other embodiments, the treatment dosage of NMDP or a salt thereof can also be administered to a mucosal membrane selected from one or more of the buccal mucosal membranes, the mucosal membrane of the respiratory tract, such as the tracheal mucosa and/or the pulmonary mucosal membrane. In a further aspect of the disclosure the treatment dosage can be administered to more than one location in the same treatment or the patient can choose the administration route on an individual basis. Acute tinnitus or Meniere's disease or a symptom of tinnitus or Meniere's disease during night can be treated with buccal administration if the nasal administration implies an irritation of the nose.

### Area Under the Curve

As used herein “peak concentration (Cₘₐₓ) of NMDP in a blood plasma”, “area under concentration vs. time curve (AUC) of NMDP in a blood plasma”, “time to maximal plasma concentration (tₘₐₓ) of NMDP in a blood plasma” are pharmacokinetic parameters known to one skilled in the art. Laursen et al., Eur. J. Endocrinology 135:309-315, 1996. The “concentration vs. time curve” measures the concentration of NMDP in a blood scrum of a subject vs. time after administration of a dosage of NMDP to the subject either by intranasal or mucosal route of administration. “Cₘₐₓ” is the maximum concentration of NMDP in the auric structures or the blood serum of a subject following a single dosage of NMDP to the subject. “tₘₐₓ” is the time to reach maximum concentration of NMDP in a blood serum or auric structures of a subject following administration of a single dosage of NMDP to the subject.

As used herein, “area under concentration vs. time curve (AUC) of NMDP in a blood plasma or auric structures” is calculated according to the linear trapezoidal rule and with addition of the residual areas. A decrease of 23% or an increase of 30% between two dosages would be detected with a probability of 90% (type II error β=10%). The “delivery rate” or “rate of absorption” is estimated by comparison of the time (tₘₐₓ) to reach the maximum concentration (Cₘₐₓ). Both Cₘₐₓ and tₘₐₓ are analyzed using non-parametric methods. Comparisons of the pharmacokinetics of intramuscular, subcutaneous, intravenous and intranasal NMDP administrations are performed by analysis of variance (ANOVA). For pair wise comparisons a Bonferroni-Holmes sequential procedure is used to evaluate significance. The dose-response relationship between the three nasal doses is estimated by regression analysis. P<0.05 is considered significant. Results are given as mean values+/−SEM.

In one embodiment, the intranasal dose of NMDP, achieves a transient serum level of between about 5 pg/mL to about 280 pg/ml, between about 10 pg/mL to about 100 pg/mL, between about 20 pg/mL to about 50 pg/mL or between 25 pg/mL to about 250 pg/mL. In intranasal formulations, the transient NMDP serum blood level achieved can be lower or higher than that typically obtained by other routes of administration. In some embodiments, the beneficial effects achieved by intranasal administration are similar to those obtained from a steady serum NMDP level of between about 5 ng/dL to about 180 ng/dL.

A comparison of the total area under concentration-time curves (AUC) or average concentrations of NMDP in subjects treated with intranasal NMDP and patients treated with NMDP by another route, such as oral, provides a basis for determining the biological equivalency of different routes of administration. Where the AUCs or average concentrations are similar, despite different routes of administration or different concentration-time profiles, the biological effect achieved is often similar. Thus, in one embodiment, the disclosure contemplates achieving by intranasal administration of the disclosed composition an average serum NMDP concentration over 24 hours of between about 5 pg/mL and about 250 pg/mL.

In one embodiment, the intranasal dose of NMDP, achieves a transient concentration level of between about 5 pg/mL to about 280 pg/ml, between about 10 pg/mL to about 100 pg/mL, between about 20 pg/mL to about 50 pg/mL or between 25 pg/mL to about 250 pg/mL in the auric structures. In intranasal formulations, the transient NMDP serum auris level achieved can be lower or higher than that typically obtained by other routes of administration.

The peak plasma concentrations achieved by intravenous administration of NMDP are associated with side effects such as depression of the respiratory system. In embodiments, the peak plasma concentrations of pharmaceutical composition are sufficient to provide the desired effect (as well as being achieved rapidly and sustained sufficiently long). Thus, the disclosure further relates to a composition wherein said administration of the no more than two dosage units has a peak plasma concentration of no less than 5% and no more than 75% of the peak plasma concentration obtained by intravenous administration of said dosage unit(s), within the treatment dosage range of from about 20 to 2500 mg, preferably a peak plasma concentration of no less than 30% and no more than 75% of the peak plasma concentration obtained by intravenous administration of said dosage unit(s), within the treatment dosage range of from about 20 to 2500 mg.

In embodiments, repeated administration of dosage units does not result in increases in peak plasma levels. In intravenous administration, repeated administration continues to elevate the plasma concentrations to undesired high levels. In some embodiments, repeated transmucosal administration of a dosage unit of NMDP or a salt thereof does not continue to elevate the plasma concentration. In some embodiments, the method is such that administration of the medicament results in a C_{max,nasal}/C_{max,iv} ratio which decreases with increasing dosage units when comparing equal amounts of NMDP delivered by both modes of administration (nasal vs. intravenous), within the treatment dosage range of from about 50 to 2500 mg.

### Formulations

The compounds, or therapeutically acceptable salts thereof, can be formulated as pharmaceutical compositions. Such compositions can be administered by inhalation spray, nasally in dosage unit formulations containing conventional nontoxic therapeutically acceptable carriers, adjuvants, and vehicles as desired. In some embodiments, the pharmaceutical compositions can be administered by oral dose forms, such as a tablet.

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

The active compounds (or therapeutically acceptable salts thereof) can be administered per se or in the form of a pharmaceutical composition wherein the active compound(s) is in admixture or mixture with one or more therapeutically acceptable carriers, excipients or diluents. Pharmaceutical compositions can be formulated in conventional manner using one or more therapeutically acceptable carriers comprising excipients and auxiliaries which facilitate processing of the active compounds into preparations which can be used therapeutically. Proper formulation is dependent upon the route of administration chosen.

Additionally, the formulation can contain conventional carriers such as plasticizers, pigments, colorants, glidants, stabilization agents, pore formers and surfactants.

Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicon dioxide, titanium oxide, magnesium aluminum silicate and powdered sugar. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone. Examples of suitable lubricants include, but are not limited to, magnesium stearate, calcium stearate, stearic acid, glycerol behenate, polyethylene glycol, talc, and mineral oil. Disintegrants can be, are not limited to, starch, sodium starch glycolate, sodium carboxymethyl starch, sodium carboxymethylcellulose, hydroxypropyl cellulose, pregelatinized starch, clays, cellulose, alginine, gums or cross-linked polymers, such as cross-linked PVP (Polyplasdone XL from GAF Chemical Corp). Stabilizers are used to inhibit or retard drug decomposition reactions which include, by way of example, oxidative reactions.

Surfactants can be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

If desired, tablets, beads, granules, or particles used for administration of the pharmaceutical composition can also contain minor amounts of nontoxic auxiliary substances such as wetting or emulsifying agents, dyes, pH buffering agents, or preservatives.

The pharmaceutical compounds can be complexed with other agents as part of therapeutic formulation. The pharmaceutical compositions can take the form of, for example, liquid formulations for nasal administration prepared in water or other aqueous vehicles can contain various suspending agents such as methylcellulose, alginates, tragacanth, pectin, kelgin, carrageenan, acacia, polyvinylpyrrolidone, and polyvinyl alcohol. The liquid formulations can also include solutions, emulsions, syrups and elixirs containing, together with the active compound(s), wetting agents, sweeteners, and coloring and flavoring agents. Various liquid and powder formulations can be prepared by conventional methods for inhalation by the patient.

In addition, combinations of immediate release compositions and delayed release/extended-release compositions can be formulated together.

In some embodiments, NMDP is formulated as the sole active pharmaceutical ingredient (API) in a dosage form. Such NMDP dosage form can be used alone or in combination therapy with one or more additional dosages containing one or more active pharmaceutical ingredients for prevention or treatment of hearing loss. In such cases, the daily dosage of NMDP is conveniently provided in a single dosage form as described herein, or can be divided amongst two, three, four or more dosages.

As noted above, the composition comprised of NMDP or a salt thereof, is mucosally administered by contacting the composition in a suitable dosage form with mucosal tissue of the vagina, nose, rectum, or mouth. In an embodiment, the composition is administered via the nasal mucosa, i.e., intranasally. The nasal mucosa provides a useful anatomical site for systemic delivery. The nasal tissue is highly vascularized, providing an attractive site for rapid and efficient absorption. The adult nasal cavity has a capacity of around 20 mL, with a large surface area of approximately 180 cm² for drug absorption, due in part to the microvilli present along the pseudostratified columnar epithelial cells of the nasal mucosa.

A nasal preparation comprised of the composition described above can take a variety of forms for administration in nasal drops, nasal spray, gel, ointment, cream, powder or suspension, using a dispenser or other device as needed. A variety of dispensers and delivery vehicles are known in the art, including single-dose ampoules, atomizers, nebulizers, pumps, nasal pads, nasal sponges, nasal capsules, and the like.

More generally, the preparation can take a solid, semi-solid, or liquid form. In the case of a solid form, the components can be mixed together by blending, tumble mixing, freeze-drying, solvent evaporation, co-grinding, spray-drying, and other techniques known in the art. Such solid-state preparations preferably provide a dry, powdery composition with particles in the range of between about 5 to about 500 microns, more preferably from 50 to 250 microns, for administration intranasally.

A semi-solid preparation suitable for intranasal administration can take the form of an aqueous or oil-based gel or ointment. For example, the components described above can be mixed with microspheres of starch, gelatin, collagen, dextran, polylactide, polyglycolide or other similar materials that are capable of forming hydrophilic gels. The microspheres can be loaded with drug, and upon administration form a gel that adheres to the nasal mucosa.

### Nasal Spray Formulations

In an embodiment, the nasal preparation is in liquid form, which can include an aqueous solution, an aqueous suspension, an oil solution, an oil suspension, or an emulsion, depending on the physicochemical properties of the composition components. The liquid preparation is administered as a nasal spray or as nasal drops, using devices known in the art, including nebulizers capable of delivering selected volumes of formulations as liquid-droplet aerosols. For example, a commercially available spray pump with a delivery volume of 50 or 100 μL is available from, for example, Valois (Congers, N.Y.) with spray tips in adult size and pediatric size. In one embodiment, the composition comprised of at least NMDP is administered intranasally via an aerosol spray in a daily volume of between about 30 to about 200 μL.

The liquid preparation can be produced by known procedures. For example, an aqueous preparation for nasal administration can be produced by dissolving, suspending, or emulsifying NMDP or a salt thereof in water, buffer, or other aqueous medium, or in an oleaginous base, such as a pharmaceutically acceptable oil like olive oil, lanoline, silicone oil, glycerine, fatty acids, and the like.

The compositions according to the present disclosure can be applied to the nasal cavity as liquids, sprays, aerosols, nebulizers or semi-solid preparations. Semisolid preparations can be on the base of gels, w/o or o/w creams or hydrophilic/lipophilic ointments. The compositions can contain molecularly dispersed (soluble, solubilized, etc.) active agent or the fine particles/crystals of the active agent. The compositions could be administered from nasal sprays, metered-dose sprays, squeeze bottles, liquid droppers, disposable one-dose droppers, nebulizers, cartridge systems with unit-dose ampoules, single-dose pumps, bi-dose pumps, multiple-dose pumps or any other device. For example, the compositions of the disclosure can be stored in/delivered from a spray or aerosol device/container as described in details in Remington's Pharmaceutical Sciences (16th edition, Chapters 83 and 92).

Regarding spray devices, it should be noted that both single (unit) dose or multiple dose systems can be used. Typically, a spray device comprises a bottle and a pump; such devices are commercially available from various sources. Typically, the volume of liquid that is dispensed in a single spray actuation is in the range of from 5 to 250 microliters/each nostril/single administration and the concentration of the active ingredient in the formulation can be readily adjusted such that one or more spray into the nostrils will comply with the dosage regimen. The present disclosure also provides a spray device or a dose cartridge for use in a nasal delivery device loaded with a composition as described above.

As used herein, nasally administering or nasal administration includes administering the compositions into nostrils of the nose to the mucous membranes of the nasal passage or nasal cavity of the mammal. Such formulations can be administered, for example, as a nasal spray, nasal inhaler, nasal drop, aerosol, propellants, pressured dispersion, aqueous aerosol, nebulizer, nasal suspension, instillation, nasal gel, nasal powder, nasal ointment and nasal cream by aid of any new or old type device. Administration of compositions of the present disclosure can also take place using a nasal tampon or nasal sponge containing the compositions.

In some embodiments, the therapeutically effective amount of NMDP is administered in a composition comprising: a carrier, a citrate buffer and a benzalkonium chloride. In some embodiments, the composition can further comprise a non-aqueous solvent. In some embodiments, the composition further comprises a surfactant. In some embodiments, the amount of the carrier can be present in an amount of about 30%, 35%, 40%, 45%, 50%, 55%, 60%, 65%, 70%, 75%, 80%, 90%, or 95% weight by weight. In some embodiments, the amount of the non-aqueous solvent can range from about 10%, about 15%, about 20%, about 25%, about 30%, about 40%, about 50%, about 60%, about 70%, about 80% or about 95% weight by weight. In some embodiments, the amount of the surfactant in the composition can range from about 0.00001%, about 0.0001%, about 0.001%, about 0.01%, about 0.1%, about 1%, about 10%, about 15%, about 20%, about 25%, about 30%, about 35%, about 40%, about 45%, about 50% or about 60%.

In some embodiments, the therapeutically effective amount of NMDP is administered in an intranasal composition that does not comprise a non-ionic surfactant.

In some embodiments, the intranasal composition that does not comprise the non-ionic surfactant comprises PEG, mPEG, water, and NMDP. In some embodiments, the intranasal composition that does not comprise the non-ionic surfactant comprises a composition that comprises about 20% to about 90%, about 30% to about 90%, about 40% to about 90%, about 50% to about 90%, about 60% to about 90%, about 70% to about 90% or about 80% to about 90% (w/w) mPEG, or mPEG 350, about 10% to about 50%, about 20% to about 50%, about 30% to about 50%, or about 40% to about 50% (w/w) PEG, or PEG400, and about 5% to about 20% about 10% to about 20%, or about 15% to about 20% (w/w) water and NMDP. In some embodiments, the concentration of NMDP in the intranasal composition that does not comprise the non-ionic surfactant is at least 10 mg/mL, at least 15 mg/mL, at least 20 mg/mL, at least 25 mg/mL, at least 30 mg/mL, at least 35 mg/mL, at least 40 mg/mL, at least 45 mg/mL, at least 50 mg/mL, at least 55 mg/mL, at least 60 mg/mL, at least 65 mg/mL, at least 70 mg/mL, at least 80 mg/mL, at least 90 mg/mL, at least 95 mg/mL, at least 100 mg/mL, at least 110 mg/mL, or greater than 110 mg/mL. In some embodiments, the therapeutically effective amount of NMDP is administered in an intranasal composition that comprises about 70% (w/w) mPEG 350, about 20% (w/w) PEG400, and about 10% (w/w) water. In some embodiments, the concentration of NMDP of the intranasal composition is at least 10 mg/mL, at least 15 mg/mL, at least 20 mg/mL, at least 25 mg/mL, at least 30 mg/mL, at least 35 mg/mL, at least 40 mg/mL, at least 45 mg/mL, at least 50 mg/mL, at least 55 mg/mL, at least 60 mg/mL, at least 65 mg/mL, at least 70 mg/mL, at least 80 mg/mL, at least 90 mg/mL, at least 95 mg/mL, at least 100 mg/mL, at least 110 mg/mL, or greater than 110 mg/mL.

In some embodiments, the intranasal composition that does not comprise a non-ionic surfactant comprises PEG, mPEG, water, ethanol, and NMDP. In some embodiments, the intranasal composition that does not comprise a non-ionic surfactant comprises a composition that comprises about 20% to about 90%, about 30% to about 90%, about 40% to about 90%, about 50% to about 90%, about 60% to about 90%, about 70% to about 90% or about 80% to about 90% (w/w) mPEG, or mPEG 350, about 10% to about 50%, about 20% to about 50%, about 30% to about 50%, or about 40% to about 50% (w/w) PEG, or PEG400, and about 5% to about 20% about 10% to about 20%, or about 15% to about 20% (w/w) water, about 1% to about 10% about 2% to about 10%, about 3% to about 10%, about 4% to about 10%, about 5% to about 10%, about 6% to about 10%, about 7% to about 10%, about 8% to about 10%, or about 9% to about 10% ethanol, and NMDP. In some embodiments, the concentration of NMDP in the intranasal composition is at least 10 mg/mL, at least 15 mg/mL, at least 20 mg/mL, at least 25 mg/mL, at least 30 mg/mL, at least 35 mg/mL, at least 40 mg/mL, at least 45 mg/mL, at least 50 mg/mL, at least 55 mg/mL, at least 60 mg/mL, at least 65 mg/mL, at least 70 mg/mL, at least 80 mg/mL, at least 90 mg/mL, at least 95 mg/mL, at least 100 mg/mL, at least 110 mg/mL, or greater than 110 mg/mL.

In some embodiments, the therapeutically effective amount of NMDP administered in a composition comprising one or more forms of a alkoxy-polyethylene glycols.

R—O—(CH2CH2O)ₙ—H, wherein, R is methyl, ethyl, n-propyl, isopropyl, or cyclopropyl. In some embodiments, the average number of oxyethylene repeating units is a number in the range from about 1 to about 25. an alkoxy-polyethylene glycol represented by Formula I:

R—O—(CH2CH2O)ₙ—H (I), wherein, R is (C1-C6)alkyl; and n, which is the average number of oxyethylene repeating units, is a number in the range of from about 1 to about 25. The term “(C1-C6)alkyl” refers to an alkyl group having between 1 and 6 carbon atoms. Representative alkyl groups include methyl, ethyl, n-propyl, isopropyl, n-butyl, sec-butyl, isobutyl, tert-butyl, pentyl, hexyl, cyclopropyl, cyclobutyl, cyclopropylmethylene, cyclopentyl, cyclobutylmethylene, cyclobutylethylene, cyclohexyl, cyclopropylpropylene, cyclobutylethylene, and cyclopentylmethylene.

In some embodiments, the ethylene glycols can be used in the form of the single compounds or as a mixture of two or more methoxy-n-ethylene glycols.

In some instances, the alkoxy-polyethylene glycol is polyethylene glycol 200 (PEG 200), polyethylene glycol 400 (PEG 400), polyethylene glycol 600 (PEG 600), polyethylene glycol 1000 (PEG 1000). The term “PEG 200” is understood to mean polyethylene glycol having an average molecular weight of about 200 daltons. The term “PEG 400” is understood to mean polyethylene glycol having an average molecular weight of about 400 daltons. The term “PEG 600” is understood to mean polyethylene glycol having an average molecular weight of about 600 daltons. The term “PEG 1000” is understood to mean polyethylene glycol having an average molecular weight of about 1000 daltons.

In certain embodiments, the alkoxy-polyethylene glycol is methoxy-polyethylene glycol 350 (mPEG 350) or is methoxy-polyethylene glycol 550 (mPEG 550) or is methoxy-polyethylene glycol 750 (mPEG 750). The term “mPEG 350” is understood to mean methoxy polyethylene glycol having an average molecular weight of about 350, and in certain embodiments the average “n,” as denoted in Formula I, is 7.2. The term “mPEG550” is understood to mean methoxy polyethylene glycol having an average molecular weight of about 550, and in certain embodiments the average “n,” as denoted in Formula I, is 11.8. The term “mPEG750” is understood to mean methoxy polyethylene glycol having an average molecular weight of about 750, and in certain embodiments the average “n,” as denoted in Formula I, is 16.3.

Certain, preferred alkoxy-polyethylene glycols include Carbowax™ mPEG 350, Carbowax™ mPEG 550 or Carbowax™ mPEG 750, which are available commercially from Dow Chemical Company. Both mPEG350 and mPEG550 are colorless liquids that are miscible with water, alcohols, such as methanol, ethanol, n-propanol, glycerol and various oils in all proportions, and have a boiling point about 155° C. It is understood that alkoxy-polyethylene glycols are known by other names, where, for example, methoxy-polyethylene glycol is also known as mono-methyl polyethylene glycol and poly(ethylene glycol) methyl ether.

In some embodiments, the composition can be optimized, for example, with respect to bioadhesion, mucoadhesion, viscosity and sprayability. For example, mPEG 350, at an equivalent concentration as PEG 200, can still solubilize a therapeutic agent but the resulting composition has a lower viscosity. In some embodiments, the lower viscosity has a positive effect on the sprayability compared with lower molecular weight PEG 200, which is important where the formulation is to be sprayed.

In some embodiments, the composition comprises components selected from Polyethyleneglycol, propylene glycol, methoxy-propylene glycol, ethanol, and water. In some embodiments, NMDP is solubilized in one or more forms of alkoxy-polyethylene glycols, to reduce viscosity. In some embodiments, the lower viscosity formulations are made into nasal spray formulations. The resulting pharmaceutical composition at a temperature of 20° C. has a viscosity in the range of about 1.5 cP to about 60 cP, or from about 2 cP to about 50 cP, or from about 3 cP to about 40 cP, or from about 4 cP to about 30 cP, or from about 5 cP to about 25 cP. In certain embodiments, the alkoxy-polyethylene glycol can comprise from about 0.1% (w/w) to about 80% (w/w), or from about 0.5% (w/w) to about 70% (w/w), of the composition. In certain other embodiments, the alkoxy-polyethylene glycol can comprise from about 5% (w/w) to about 80% (w/w), or from about 30% (w/w) to about 75% (w/w) or from about 40% (w/w) to about 70% (w/w), of the composition. For certain hydrophilic drugs, the alkoxy-polyethylene glycol can comprise from about 0.1% (w/w) to about 80% (w/w), or from about 0.5% (w/w) to about 70% (w/w), or from about 1% (w/w) to about 60% of the composition. For certain lipophilic drugs, the alkoxy-polyethylene glycol can comprise from about 1% (w/w) to about 80% (w/w), or from about 2% (w/w) to about 65% (w/w), or from about 5% (w/w) to about 50% of the composition. Furthermore, the therapeutic agent can comprise from about 0.001% (w/v) to about 20% (w/v) of the composition, or from about 0.1% (w/v) to about 10% (w/v) of the composition.

The pharmaceutical composition can have a pH in the range of from about 4.5 to about 8.5, or from about 4.5 to about 7.5, or from about 4.5 to about 6.5, or from about 5.5 to about 8.5, or from about 6.5 to about 8.5, or from about 5.5 to about 7.5.

By using one or more of the alkoxy-polyethylene glycols described herein, the resulting pharmaceutical compositions can be optimized, for example, with respect to bioadhesion, viscosity and sprayability. For example, mPEG 350, at an equivalent concentration as PEG 200, can still solubilize a therapeutic agent but the resulting composition has a lower viscosity. As a result, this substitution has a surprisingly positive effect on the sprayability compared with lower molecular weight PEG 200, which is important where the formulation is to be sprayed.

It will be appreciated that excipients necessary for formulation, stability, and/or bioavailability can be included in the preparation. Exemplary excipients include sugars (glucose, sorbitol, mannitol, sucrose), uptake enhancers (chitosan), thickening agents and stability enhancers (celluloses, polyvinyl pyrrolidone, starch, etc.), buffers, preservatives, and/or acids and bases to adjust the pH.

In an embodiment, an absorption promoting component is included. Exemplary absorption promoting components include surfactant acids, such as cholic acid, glycocholic acid, taurocholic acid, and other cholic acid derivatives, chitosan and cyclodextrins. In some embodiments, a cyclodextrin is included in the preparation.

Exemplary surfactants, include, for example nonoxynol, octoxynol, tweens, spans, sodium lauryl sulfate, and sorbitan monopalmitate. Exemplary absorption promoters include, for example, bile salts and derivatives thereof, fusidic acid and derivatives thereof, oleic acid, lecithin, lysolechitins, dodecanoyl phosphatidylcholine (DDPC), sucrose monododecanoate, n-dodecyl-β-D-maltopyranoside, pectin, chitosan, α-, β- and γ-cyclodextrins and derivatives thereof, pegylated caprylic-/capric glycerides and derivatives thereof, such as, Softigen and Labrasol. Exemplary water absorbing polymers include, for example, polyethylene glycols having an average molecular weight ranging from 200 to 7500, propylene glycol, or mixtures thereof, or single ethylene glycols such as tetraethylene glycol and pentaethylene glycol. Exemplary alcohols include, for example, ethanol, isopropyl alcohol. Exemplary lipids include, for example, vegetable oil, soybean oil, peanut oil, coconut oil, maize oil, olive oil, sunflower oil, monoglycerides, diglycerides, mono/diglycerides, mono/di/triglycerides. Exemplary osmotic pressure controlling agents include, for example, glycerol, dextrose, maltose, sucrose, mannitol, xylitol, various salts (for example, sodium chloride). Exemplary pH-controlling agents include, for example, buffers, acids (for example, nitric acid, phosphoric acid, or acetic acid). Exemplary preservatives include, for example, methyl paraoxybenzoate, phenyl ethyl alcohol or benzoic acid. Exemplary propellants, include, for example, butane or air displacement such as nitrogen. Excipients adjusting the HLB of the formulation include, for example, Tween 20, 25, 40, 45, 65, 85, Span 20-80, Brij 30-98, acacia. Exemplary enzyme inhibitors include, for example aprotinin and other peptidase inhibitors, diisopropylfluorophosphate (DFP), carbopol. Exemplary stabilizers include, for example, cyclodextrins.

It can be helpful to include additional compounds that enhance the solubility of the therapeutic agent. Examples of such solubilizers include, for example, alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, pegylated-mono/di-caprylic/capric glycerides, polyvinylalcohol, hydroxypropyl methylcellulose and other cellulose derivatives, cyclodextrins (for example, α-, β-, or γ-cyclodextrins) and cyclodextrin derivatives; ethers of polyethylene glycols having an average molecular weight of about 200 to about 6000 or tetrahydrofurfuryl alcohol PEG ether (glycofurol, available commercially from BASF under the trade name Tetraglycol); surfactants, such as, sodium lauryl sulfate, oleic acid, linoleic acid, monoolein, lecithin, lysolecithin, deoxycholate, taurodeoxycholate, glycochenodeoxycholate, polyoxyethylene X-lauryl ether, where X is from 9 to 20, sodium tauro-24,25-dihydrofusidate, polyoxyethylene ether, polyoxyethylene sorbitan esters, p-t-octylphenoxypolyoxyethylene, N-lauryl-β-D-maltopyranoside, 1-dodecylazacycloheptane-2-azone; amides, such as, 2-pyrrolidone, 2-piperidone, caprolactam, N-alkylpyrrolidone, N-hydroxyalkylpyrrolidone, N-alkylpiperidone, N-alkylcaprolactam, dimethylacetamide, and polyvinylpyrrolidone; esters, such as, ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, caprolactone and isomers thereof, valerolactone and isomers thereof, β-butyrolactone and isomers thereof; and other solubilizers known in the art, such as dimethyl acetamide, dimethyl isosorbide (Arlasolve DMI (ICI)), N-methylpyrrolidones (Pharmasolve (ISP)), monooctanoin, and diethylene glycol monoethyl ether (available from Gattefosse under the trade name Transcutol).

Preferred additional solubilizers include triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-1000, PEG 300, PEG 400, Transcutol, and dimethyl isosorbide, sorbitol, glycerol, triacetin, glycofurol and propylene glycol. Typically, the solubilizer, if present, is present in an amount of from about 0.1% (w/v) to about 50% (w/v), from about 1% (w/v) to about 40% (w/v) or from about 2% (w/v) to about 25% (w/v). In addition, the liquid pharmaceutical composition can comprise water, for example, from about 2% (w/v) to about 99% (w/v), from about 10% (w/v) to about 95% (w/v), or from about 20% (w/v) to about 90% (w/v), of the liquid composition.

As discussed, the composition can comprise a preservative. In addition or in the alternative, the composition can be sterilized. Sterilization can be achieved by filter sterilization, autoclaving, exposure to ionizing radiation, for example, gamma radiation, UV irradiation, and chemical sterilization. In one embodiment, the sterile composition has a sterility assurance level of at least about 103. The resulting liquid compositions preferably are stable at room temperature, such that less than 5%, 4%, 3%, 2% or 1% by weight of the therapeutic agent degrades after storage for 30 days, or more preferably 6 months, at 20° C.

In addition, the formulations can also include a sweetener or flavoring agent. Exemplary sweeteners or flavoring agents include, for example, acacia syrup, acesulfame potassium, anethole, anise oil, aromatic elixir, aspartame, benzaldehyde, benzaldehyde elixir, cyclodextrins, caraway, caraway oil, cardamom oil, cardamom seed, cardamom spirit, cardamom tincture, cherry juice, cherry syrup, cinnamon, cinnamon oil, cinnamon water, citric acid, citric acid syrup, clove oil, cocoa, cocoa syrup, coriander oil, dextrose, eriodictyol, eriodictyol fluid extract, eriodictyol syrup, aromatic, ethylacetate, ethyl vanillin, fennel oil, ginger, ginger fluid extract, ginger oleoresin, glucose, sugar, maltodextrin, glycerin, glycyrrhiza, glycyrrhiza elixir, glycyrrhiza extract, glycyrrhiza extract pure, glycyrrhiza fluid extract, glycyrrhiza syrup, honey, iso-alcoholic elixir, lavender oil, lemon oil, lemon tincture, maltodextrin, maltose, mannitol, methyl salicylate, menthol, nutmeg oil, orange bitter, elixir, orange bitter, oil, orange flower oil, orange flower water, orange oil, orange peel, bitter, orange peel sweet, tincture, orange spirit, orange syrup, peppermint, peppermint oil, peppermint spirit, peppermint water, phenylethyl alcohol, raspberry juice, raspberry syrup, rosemary oil, rose oil, rose water, saccharin, saccharin calcium, saccharin sodium, sarsaparilla syrup, sarsaparilla compound, sorbitol solution, spearmint, spearmint oil, sucrose, sucralose, syrup, thyme oil, tolu balsam, tolu balsam syrup, wintergreen oil, vanilla, vanilla tincture, vanillin, wild cherry syrup, xylitol, or combinations thereof.

In addition, the formulations optionally can contain taste masking agents. Exemplary masking agents include, for example, cyclodextrins, cyclodextrin emulsions, cyclodextrin particles, cyclodextrin complexes, or combinations thereof.

### Dry Powder Compositions

In specific embodiments, the NMDP compositions prepared according to the herein described methods, can be designed for desired NMDP levels. For example, in one embodiment, a level of about 22.2% w/w NMDP hydrochloride or a hydrate thereof in the form of microspheres when said microspheres are separated and dis-agglomerated by at least 77.8% of disaggregating particles, e.g., lactose monohydrate. In some embodiments, the final composition can be then introduced into a disposable dose device, as described below, to provide a dose of from about 50 to about 2500 mg NMDP hydrochloride upon single intranasal administration.

The pharmaceutical composition according to the present disclosure can be contained in disposable dose units for intranasal administration, providing predetermined metered dose of NMDP or a salt thereof. In one example of a disposable unit, a Unit Dose Powder Device (UDS), manufactured by Aptar Pharma. Devices of this type for powder spraying can be used for systemic delivery of small and accurately metered doses of NMDP formulations by patients or caregivers who are not healthcare professionals or medically trained.

The present disclosure further relates to a dose unit form (also referred to as dose unit device, dose device or drug device), specifically a disposable dose unit form, for intranasal administration to a subject of a single dose of a\the pharmaceutical composition according to the present disclosure, which comprises as active ingredient an opioid receptor antagonist, specifically NMDP or pharmaceutically acceptable salts thereof such as NMDP hydrochloride, wherein the dose unit is loaded with a predetermined dose of the composition and provides the subject with a metered dose the pharmaceutically active ingredient comprised in the composition. In some embodiments, the dose unit forms are storage stable. In addition to the dose unit devices described above, the NMDP composition of the present disclosure can be administered using syringe-driven device and pump-driven spraying atomizers. Bi-dose and multiple-dose administration devices are also contemplated within the scope of the present disclosure. In some embodiments, the NMDP contained in the dose devices of the present disclosure are retained in amorphous form.

Compositions according to this aspect of the disclosure which contain NMDP or pharmaceutically acceptable salt thereof as the active opioid receptor antagonist and dose units thereof are also referred to herein as NMDP compositions, respectively NMDP dose units.

The NMDP compositions of the present disclosure are particularly intended for treatment of tinnitus or Meniere's disease. Treatment of tinnitus or Meniere's disease as referred to herein is to be taken to mean alleviating or reversing the effects of the tinnitus or Meniere's disease, as well as symptoms associated therewith such as hearing loss, aural pressure, among others. The NMDP compositions and dose units thereof according to the present disclosure have been shown to be significantly effective.

In some embodiments, the NMDP intranasal powder formulation in accordance with the present disclosure is administered for intranasal administration, a high proportion of the NMDP particle of at least 25%, at least 30%, at least 35%, at least 40%, at least 45%, at least 50%, at least 50%, at least 60%, at least 70%, at least 80% or at least 86% reach the nasal turbinates region. In some embodiments, the NMDP intranasal powder formulation in accordance with the present disclosure is administered for intranasal administration, a high proportion of the NMDP particle of at least 25%, at least 30%, at least 35%, at least 40%, at least 45%, at least 50%, at least 50%, at least 60%, at least 70%, at least 80% or at least 86% reach the auric region. In some embodiments, about 15%, about 20%, about 25%, about 30% or about 35% are in the middle part of the olfactory area. In some embodiments, less than 10% of the NMDP particles are in the nose and less than 1% reach the lungs, providing at treated subject with effective amounts of NMDP and improved therapeutic effect.

Provided herein is a kit for the treatment/reversal of tinnitus or Meniere's disease. The kit comprises at least one dose unit of NMDP powder composition as disclosed herein and instructions for use. The dose unit can be single-dose, bi-dose or multiple-dose unit.

In one embodiment, a pharmaceutical composition in a form of dry powder for intranasal (nose-to-brain) N2B administration to a patient in need thereof comprises solid particles of NMDP and solid particles of a diluent, said pharmaceutical composition being substantially free of excipients other than the solid diluent, wherein said pharmaceutical composition having at least 90% of the particles of NMDP with a mean particle size of 10-30 microns and less than 10% of the particles of said at least one active agent with a mean particle size of about 5-50 microns, and having the particles of said diluent with a mean particle size of 50-200 microns.

In some embodiments, the composition can be comprised of a solid diluent, such as lactose monohydrate or a lactose functional analogue. In another embodiment, a pharmaceutical composition in a form of dry powder for intranasal administration by transmucosal systemic delivery via the upper nose mucosa (the turbinate and lymphoid tissues located at the back of the nasal cavity) of a composition comprises NMDP or a salt thereof having a mean particle size in the range of 10-30 microns. In some embodiments, at least 90% of its particles have a mean particles size of not less than 5 microns and not more than 30 microns, and a diluent having a mean particle size in the range of 50-200 microns. As noted above, the diluent is also used for preventing aggregation of the dry powder particles containing NMDP or a salt thereof.

The composition of the embodiments can be delivered by any one of the known in the art nasal devices, such as pressurized devices, dry powder sprayers or bi-directional nasal devices. Multi-dose devices as well as single-dose devices can be used.

The NMDP content of the composition of the embodiments can be adjusted so as to provide the total dose of the drug required to achieve the therapeutic effect as a single dose in a single nostril. The drug administration can be repeated in the second nostril in order to double the amount of the active material. Stability of the composition of the embodiments on storage can be determined under accelerated and ambient conditions.

In some embodiments, the compositions herein can comprise NMDP and a diluent, such as lactose or a lactose functional analogue, and are substantially free of other excipients, such as surfactants, lipid agents, solvents or propellants. The solid diluent of the embodiments can be selected from lactose monohydrate or a lactose monohydrate functional analogue, such as lactose, cellulose and derivatives, starch and derivatives, dextrose, sorbitol, mannitol, maltitol, xylitol or mixtures thereof. The solid diluent can be lactose monohydrate.

Lactose can be present in the form of α-lactose monohydrate, anhydrous β-lactose or amorphous lactose. The pharmaceutical composition of the embodiments can further comprise one or more pharmaceutically acceptable diluents, excipients or both. The pharmaceutical composition of the embodiments can be prepared in the form of a powder, simple powder mixtures, powder microspheres, coated powder microspheres, liposomal dispersions or combinations thereof.

In some embodiments of the present disclosure the therapeutically effective therapeutically effective amount of NMDP or a salt thereof, is equivalent to about 10 mg, administered intranasally as a single dose, to about 50 mg or 2500 mg of NMDP, optionally administered in several doses. In some embodiments, the therapeutically effective amount is equivalent to about 3, 4, 5, 6, 7, or 8 to about 9, 10, 15, 20, 25, 30, 35, 40, 50, 60, 80, 90, 100, 120, 150, 200, or 2500 mg, respectively, of NMDP. In some embodiments, the NMDP or a salt thereof is the only pharmaceutically active compound in pharmaceutical composition. In some embodiments, the methods of treatment with NMDP powder compositions and formulations according to the present disclosure can provide for a plasma concentration versus time curve of said NMDP in said patient of a Tₘₐₓ between about 0.13 and about 0.75 hours, for example Tₘₐₓ of 0.25 h. The methods of treatment with NMDP powder compositions and formulations according to the present disclosure can provide mean maximum plasma concentration of NMDP of about 20 ng/mL to about 25, 30, 40, 45, 50, 70, 90, 100, 120, 150, 180, 200, 250, 500, 1000 ng/ml or 20 ng/ml, 50, 100, 150, or 250 ng/mL within 15 minutes of administration. The methods of treatment with NMDP powder compositions and formulations according to the present disclosure can provide mean maximum concentration of NMDP of about 20 pg/mL to about 25, 30, 40, 45, 50, 70, 90, 100, 120, 150, 180, 200, 250, 500, 1000 ng/ml or 20 ng/ml, 50, 100, 150, or 250 ng/ml within 15 minutes of administration in the auric structures.

The device used for the intranasal delivery of the compositions of the embodiments can be engineered so as to provide the appropriate plume geometry and spray pattern of initial and stored compositions. In some embodiments, these compositions can have a narrow particle size distribution with median diameter between 5 to 50 microns.

### Liposomal Compositions

Provided herein is a method of administering NMDP or a salt thereof to a patient in need thereof, which method comprises the intranasal administration of a composition comprising a therapeutically effective amount of NMDP or a salt thereof, phospholipids, one or more C2-C4 alcohols and water, wherein the concentrations of the phospholipids and the one or more alcohols in the composition are in the ranges of 0.2 to 70% and 10 to 70% by weight, respectively. In some embodiments, the water content of said composition is 10%, 20%, 30%, 40%, 50%, or 60% by weight, the phospholipids forming vesicles in said composition. In some embodiments, the concentration of NMDP or a salt thereof are in the range of 0.5 to 25% by weight.

Provided herein is an aqueous composition comprising NMDP or a salt thereof, which contains phospholipids in a concentration of 0.2 to 50% by weight, in combination with one or more short chain alcohols. In some embodiments, the weight concentration of water is at least 30% by weight. In some embodiments, the weight concentration of the alcohol(s) is in the range between 10 to 50% by weight, can be adapted for use as an intranasal drug delivery vehicle. In some embodiments, the concentration of NMDP or a salt thereof in the composition are in the range of 0.5 to 25% by weight.

Accordingly, in one aspect, described here is a use of a vesicular composition comprising NMDP or a salt thereof, and a phospholipid, one or more C2-C4 alcohols and water for intranasal administration for the treatment of one or more symptoms of tinnitus or Meniere's disease in a patient in need thereof. In some embodiments, the concentration of wherein the concentrations of the phospholipid and the one or more alcohols in the composition are in the ranges of 0.2 to 50% and 10 to 50% by weight, respectively, and the water content of the composition is between 10-50% by weight. In some embodiments, the concentration of NMDP or a salt thereof are in the range of 0.5 to 25% by weight.

Phospholipids suitable for use in the preparation of the composition according to the present disclosure include phosphatidylcholine (PC), hydrogenated phosphatidylcholine, phosphatidic acid (PA), phosphatidylserine (PS), phosphatidylethanolamine (PE), phosphatidylglycerol (PPG) and phosphatidylinositol (PL). The chemical structure of phospholipids that can be used according to the present disclosure is described in U.S. Pat. No. 4,614,730, which is incorporated herein by reference. Preferably, the phospholipids are present in the composition of the disclosure for delivering NMDP intranasally for treatment of tinnitus or Meniere's disease is at a concentration of 0.5 to 15% by weight.

The term C2-C4 alcohols, as used herein, refers to alkanols containing two, three or four carbon atoms. The alcohols to be used according to the present disclosure specifically include ethanol, 1-propanol, isopropyl alcohol and tert-butyl alcohol. In some embodiments, the concentration of ethanol in the composition is in the range of 1% to 20%, 30%, 40%, 50%, 60% or 70% by weight. According to an embodiment of the disclosure, the composition further comprises one or more water miscible polyols, and especially glycols (1,2-diols, such as ethylene glycol and propylene glycol), at a concentration of 1 to 30%, 40%, or 50% by weight.

The compositions can be prepared by mixing together the various components, namely, water, phospholipids, one or more C2-C4 alcohols (and possibly also one or more polyols) and NMDP or a salt thereof under conditions that allow the formation of vesicles. In some embodiments, the compositions can be conveniently prepared by dissolving the phospholipids in the alcohol (or in the alcohol/glycol mixture), followed by the addition of NMDP or a salt thereof, either in the form of an aqueous solution thereof or in a solid form, with a subsequent addition of water. Alternatively, a dispersion of the phospholipids and NMDP or a salt thereof in water is prepared, into which the alcohol, optionally together with polyol (e.g., a mixture of ethanol and propylene glycol) are added with stirring, possibly under heating.

It is also possible to first prepare freeze-dried lipid vesicles having the active ingredient encapsulated therein, and subsequently dispersing the same in a mixture of water, the C2-C4 alcohol and optionally polyol.

In some embodiments, the size of the vesicles can range between 50 nm to few microns, and more specifically, up to 5 μm. In some embodiments, the composition according to the present disclosure can include additional excipients that are well known in the art, such as surfactants, preservatives, thickening agents, co-solvents, adhesives, antioxidants, buffers, viscosity and absorption enhancing agents and agents capable of adjusting the pH and osmolarity of the formulation. Additional excipients that can be used with the compositions and methods herein are described elsewhere in this specification.

In another aspect, the disclosure provides a method of administering an active pharmaceutical ingredient to a patient in need thereof, which method comprises the intranasal administration of a vesicular composition comprising a therapeutically effective amount of the ingredient, phospholipids, one or more C2-C4 alcohols and water, wherein the concentrations of the phospholipids and the one or more alcohols in the composition are in the ranges of 0.2 to 10% and 12 to 30% by weight, respectively, with the water content of the composition being not less than 20%, and preferably not less than 30% by weight.

### Sustained Release Formulations

In some aspects, the present disclosure describes a sustained NMDP delivery method and dosage form for treatment of symptoms related to tinnitus or Meniere's disease in mammalian subjects. In some embodiments, it is envisioned that the mucosal delivery of NMDP is formulated with one or more mucosal delivery-enhancing agents wherein NMDP dosage release is substantially normalized and/or sustained for an effective delivery period ranging from about 0.1 to about 2.0 hours; from about 0.4 to about 1.5 hours; from about 0.7 to about 1.5 hours; or from about 0.8 to about 1.0 hours; following mucosal administration. The sustained release of NMDP achieved can be facilitated by repeated administration of exogenous NMDP utilizing methods and compositions of the present disclosure.

In some embodiments, mucosal delivery-enhancing agents of the present disclosure can yield an effective increase in delivery, e.g., an increase in the maximal plasma concentration (Cmax) or Cmax in the auric structures to enhance the therapeutic activity of mucosally-administered NMDP. Another factor affecting therapeutic activity of NMDP in the auric structures for the treatment of tinnitus or Meniere's disease is residence time (RT). In some embodiments, sustained release-enhancing agents, in combination with intranasal delivery-enhancing agents, increase Cmax and increase residence time (RT) of NMDP in the auric structures. An increase in residence time at the mucosal delivery site (e.g., nasal mucosa), at the therapeutic site (e.g., auric structures) and/or systemic circulation are contemplated herein. In some embodiments, polymeric delivery vehicles and other agents and methods of the present disclosure that yield sustained release-enhancing formulations, for example, can include polyethylene glycol (PEG).

### Semi-Solid and Viscous Gel Formulations

In some of the methods contemplated herein, the intranasal NMDP nasal pharmaceutical composition is applied onto the outer lateral wall (the opposite side of the nasal septum) of the nostril cavity of each nostril, preferably the cartilage of the lateral outer wall of each nostril cavity Is located locally at about the middle to about the top of the outer wall (opposite the nasal septum) immediately below the portion. When the deposition of the nasal pharmaceutical composition is completed within each nostril of the nose, the external nose is gently and cautiously squeezed out, and/or the subject is rubbed, so that the deposited nasal pharmaceutical composition is ready for sustained release of the NMDP by maintaining contact with mucous membranes in the nasal cavity. The dosage of a typical NMDP nasal pharmaceutical composition deposited upon nasal cavity application is from about 50 to about 150 microliters per non-cavitation, and preferably about 100 microliters per nasal cavity.

In carrying out the method of the disclosure, about 50 microliters to about 150 microliters of the pharmaceutical composition of the present disclosure can be administered to each nostril of the subject daily, for example, 1, 2, 3, 4 weeks, Three, four, five, six, seven, eight, or intermittently, for example, two, three, four, five or six consecutive months or more, Or once a week, twice or three times, or on demand for the hearing disorders

Methods of the current disclosure include aspects of dosage concentrations of the intranasal NMDP composition, the number of daily administrations, the duration of treatment, the non-oral method and in some embodiments, a pre-filled, multi-dose applicator system delivering an effective dosage level of the NMDP or mixtures thereof in an intranasal composition delivering any number of applications and effective amounts of NMDP or mixtures thereof, daily, weekly, monthly or yearly,

In some embodiments, the nasal pharmaceutical composition of the present disclosure has a viscosity of at least about 500 cps (e.g., cP) and is in the range of from about 500 cps to about 100,000 cps prior to administration of the relevant thixotropic properties with a portion of the novel nasal pharmaceutical composition. In some embodiments, the viscosity is from about 1000 cps to about 75,000 cps, from about 2500 cps to about 50,000 cps, and from about 2500 cps to about 50,000 cps, prior to administration or pump operation in terms of thixotropic properties associated with a portion of the novel nasal pharmaceutical composition can range from about 5,000 cps to about 25,000 cps. In some embodiments, the composition has a viscosity of about 10 to about 100 cps, about 10 to about 90 cps, about 10 to about 80 cps, about 10 to about 70 cps, about 10 to about 60 cps or about 10 to about 50 cps.

In some embodiments, the applicator system of the present disclosure is, for example, an airless fluid, an immersion-tube fluid distribution system or pump, or any other system suitable for carrying out the method of the present disclosure. The applicator system or pump includes a chamber pre-filled with multiple doses of the inventive intranasal NMDP gel, closed by, for example, an actuator nozzle. The actuator nozzle can comprise an outlet channel and a tip, wherein the actuator nozzle comprises: (a) a coherent delivery of a uniform volume of the intranasal NMDP gel of the present disclosure during application of the nasal cavity around the nasal cavity of the patient, and (b) is configured to conform to the inner surface of the user's nostrils for deposition at each indicated location in the patient's nostril contemplated by the novel method and teachings of the disclosure. Preferably, when inserted into the nasal cavity, the pump design is configured to ensure that the nasal tip is properly positioned within the nasal cavity so that the gel is dispensed within the proper location within the nasal cavity when the gel is dispensed.

The oily gel, emulsion or cream can be applied to the nose approximately 1 inch inside the opening (non-air) using a suitably designed and secure dispenser tip to reach the nose and attach to the container. The tip is preferably rounded to prevent injury. The nose will then be massaged to spread the composition into a thin film of non-hollow interior, which will help absorb the active ingredient into the mucosal tissue.

Examples of pre-filled, multi-dose applicator systems include, for example, (a) Ursatec, Verpackung-GmbH, Schillerstr. Wendel, COMOD system available from Germany, (b), Airlessystems, RD 149 27380 Charleval, France or 250 North Route 303 Congers, Albion or digital airless available from NY 10950 applicator system, (c) a nasal applicator from Neopac, The Tube, Hoffmann Neopac AG, Burgdorfstrasse 22, Postfach, 3672 Oberdiessbach, Switzerland, or (d) a cannabinoid pharmaceutical composition.

Preferably, the intranasal NMDP pharmaceutical composition is filled into a non-preservative, airless multi-dose device capable of accurately delivering the capacity of the NMDP pharmaceutical composition at a higher viscosity.

According to a particular embodiment, the composition comprises: (1) a NMDP therapeutic active; (2) oily vehicles; and (3) a wetting agent, or a mixture of a wetting agent and/or a pharmaceutically acceptable surfactant or a mixture of surfactants.

According to certain embodiments, the oily vehicle is recognized as one or more pharmaceutically acceptable, generally safe lipids.

According to a particular embodiment, the oily vehicle is selected from the group consisting of pharmaceutically acceptable vegetable oils, monoglycerides, diglycerides, sucrose acetate isobutyrate (SAIB), synthetic triglycerides, and combinations thereof. According to a particular embodiment, the pharmaceutically acceptable vegetable oils are selected from the group consisting of almond oil sweets (Prunus dulcis), almond oil virgin (Prunus amid glarus), aroevera oil (aroebabadensis), apricot kernel oil (fruit Pandanus armenia car), argan oil (are the California RY labor), avocado oil (PERE Seah Americana), apricot oil (fruity Taunus armenia car), amra oil (preamble Rica operational during the day lease), borage oil (swabbing Opie during the day lease), black seed oil (age Gela sativa), carrot oil (Dow Syracuse Caro L), coconut oil (nose Syracuse nusi Pera), corn oil, cucumber oil (Cucumis sativa), tea ulmu gras oil (hydroxy-no kapuseu Wiig Tia taunus), emu oil (draw My funny nobae—Hall Grandi kids), moon yikkot oil (Oe Bruno Terra Bien Nice), linseed oil (rineom Wuxi City Tatiana stopped), grapeseed oil (Vitus Beanie Blow), hazelnut oil (ahbekeu Kana), Yo bar purified oil (deep diamond cyano difference norbornene-cis), Moringa five days Blow to raise (Moringa), e Lula oil ('s Klee Rocca ria non LEA), wheat germ oil, tree tikum No Les, macadamia oil (Macadamia terni polyamic), musk melon oil (Kubu Miss melon), Maersk oil (Abel Moss cheoseu Moss primary Tooth), mustard oil's oil (Azadi lakh other indica), olive oil (oleic Ah Europa), peach kernel oil (fruity Taunus pere Chicago), peanut oil (arachis hypo geah), pomegranate oil, Fu Nika Gras natum, program soral Leah oil (program soral Leah Cora Come polyamic), Supreme rose oil (Oe Bruno Terra Bien Needle), papaya seed (K Rica papaya), Rosehip oil (Rosa Ruby-based labor), a safflower oil, sesame seed (refined) (Cesar stopped Indy Com), the living tree oil (Hippo wave Lam Noi death) Both oils (Soya Heath the blood), sunflower oil (Heli no tooth should Taunus), Sweet Almond Oil (fruit pandanus Ami month Russ Barr moon Syracuse), sweet cherry kernel oil (fruity Taunus Oh Away) walnut oil (jugeul Lance Reg A), watermelon oil (Citrus vulgaris).

According to certain embodiments, the oily vehicle comprises castor oil and/or sesame oil and/or SAIB.

According to a particular embodiment, a mixture of wetting agent or wetting agent and/or a mixture of pharmaceutically acceptable surfactant or surfactant is selected from the group consisting of polysorbate, polyoxyethylene hydrogenated vegetable oil, polyoxyethylene vegetable oil; Polyoxyethylene sorbitan fatty acid esters; Polyoxyethylene-polyoxypropylene block copolymers, Polyglycerol fatty acid esters, Polyoxyethylene glycerides; Polyoxyethylene sterol, or derivatives or analogues thereof, A reaction mixture of at least one member of the group consisting of polyols and fatty acids, glycerides, vegetable oils, hydrogenated vegetable oils, fractionated oils and sterols; Tocopheryl polyethylene glycol succinate; Sugar esters; Sugar ether; Sucrose glyceride; Alkyl glucoside; Alkyl maltoside; Alkyl thioglycosides; Lauryl Macrogol glyceride; Polyoxyethylene alkyl ethers; Polyoxyethylene alkylphenol; Polyethylene glycol fatty acid esters; Polyethylene glycol glycerol fatty acid esters; Polyoxyethylene sorbitan fatty acid esters, Polyoxyethylene-polyoxypropylene block copolymers such as Poloxamer-108, 188, 217, 238, 288, 338, 407, 124, 182, 183, 212, 331, or 335, or combinations thereof, Ionic hydrophilic surfactants such as sodium dodecyl sulfate or sodium docusate; Bile acid; Cholic acid; Deoxycholic acid; Chenodeoxycholic acid; A salt thereof, a salt thereof, and a mixture thereof.

According to a particular embodiment, the composition comprises a rheology modifier such as colloidal silica, silicate, alumina, high molecular weight polymer or solid/wax materials, beeswax, alumina, silica, silicates and high melting point wax and/or cetostearyl alcohol.

According to a particular embodiment, the composition further comprises a mineral, an osmotic complement, a thickener and/or a hydrophilic polymer. According to a particular embodiment, the hydrophilic polymer is selected from the group consisting of HPMC, HPC, sodium CMC, sodium CMC and MCC, xanthan gum, guar gum, acacia gum, natural gums such as tragacanth gum, corn starch, potato starch, &Lt;/RTI &gt; and starch, such as starch. According to a particular embodiment, the surfactant is selected from the group consisting of glycol distearate, sorbitan trioleate, propylene glycol isostearate, glycol stearate, sorbitan sesquioleate, lecithin, sorbitan oleate, sorbitan monostearate NF, Sorbitan stearate, sorbitan isostearate, stearates-2, olet-2, glyceryl laurate, ceNMDPh-2, PEG-30 dipolyhydroxy stearate, glyceryl stearate SE, sorbitan PEG-8 sorbitan laurate, sorbitan laurate, sorbitan laurate, sorbitan laurate, sorbitan monolaurate, sorbitan monolaurate, sorbitan monolaurate, Such as Labrafil M1944CS, Laureth-4, PEG-7 glyceryl cocoate, PEG-20 almond glycerides, polyoxyethylene glycerides, PEG-25 hydrogenated castor oil, stearamide MEA, glyceryl stearate (and) PEG-100 stearate, polysorbate 85, PEG-7 olivate, cetearyl glucoside, stearamide MEA, PEG-10, oleth-10/polyoxyl 10 oleyl ether NF, Ceteth-10, PEG-8 laurate, cocamide MEA, polysorbate 60 NF, Polysorbate 60, polysorbate 80, isostearate-20, PEG-60 almond glyceride, PEG-20 methyl glucose sesquistearate, ceteareth-20, oleate-20, Steareth-20, Steareth-21, Steareth-21, Ceteth-20, and Steareth-100.

### Excipients and Other Components in the Formulation

Within various aspects of this disclosure, improved nasal mucosal delivery formulations and methods are provided that allow for the delivery of an NMDP and/or other therapeutic agents across a mucosal barrier (e.g., mucosal surface) between administration and one or more selected target sites. Certain formulations can be specifically adapted for a selected target cell, tissue or organ, or even a particular disease state. In other aspects of the instant disclosure, improved nasal delivery formulations and methods provide for efficient, selective endo- or transcytosis of NMDP specifically routed along a defined intracellular or intercellular pathway. As appreciated herein, the NMDP can be efficiently loaded at an effective concentration in a carrier or other delivery vehicle, which is then administered and maintained in a stabilized format when, for example, administered to the nasal mucosa and/or during passage through one or more intracellular compartments and/or membranes to a target site for drug action (e.g., the blood stream or a defined tissue, organ, or extracellular compartment). The NMDP can be provided in a delivery vehicle or otherwise modified (e.g., in the form of a prodrug), wherein release or activation of the NMDP is triggered by a physiological stimulus (e.g., pH change, lysosomal enzymes, etc.) In certain aspects, the NMDP can be pharmacologically inactive until it reaches its target site for activity. The NMDP and other formulation components are non-toxic (or reduce toxicity to an acceptable amount) and non-immunogenic. In this context, carriers and other formulation components are generally selected for their ability to be rapidly degraded and/or excreted under physiological conditions. At the same time, formulations are chemically and physically stable in dosage form for effective storage.

Within the mucosal delivery formulations and methods of this disclosure, the NMDP is frequently combined or coordinately administered with a suitable carrier or vehicle for mucosal delivery. As used herein, the term “carrier” includes pharmaceutically acceptable solid or liquid filler, diluent or encapsulating material. As used herein, a carrier can be a mucosal delivery enhancing agent.

A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents (e.g., a thickener), tonicity agents, wetting agents or other biocompatible materials. As disclosed herein, humectants include, but are not limited to, propylene glycol, glycerine, glyceryl triacetate, a polyol, a polymeric polyol, lactic acid, and urea. Within this disclosure, pharmaceutical formulations can contain one humectant or any combination or mixture of more than one humectant.

Solubilizing agents included in the compositions herein, can include, cyclodextrin, hydroxypropyl-β-cyclodextrin, sulfobutylether-β-cyclodextrin and methyl-β-cyclodextrin. Such solubilizing agents can be used in a pharmaceutical formulation alone or in any mixture or combination of more than one solubilizing agent. Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline, acetate, glycine, histidine, arginine, glutamate, lysine, methionine, lactate, formate, and glycolate; Ringer's solution, ethyl alcohol and phosphate buffer solutions, as well as other non-toxic compatible substances used in pharmaceutical formulations.

In some embodiments, the pharmaceutical formulations for the transmucosal, for examples, intranasal delivery of NMDP or a salt thereof, set forth herein can include any one buffering agent or any combination or mixture of more than one buffering agent. A buffering agent can have a pKa ranging from about 5 to about 9, or from about 6 to about 8. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), ethylene glycol tetraacetic acid, (EGTA), sorbitol, tartaric acid, phosphoric acid and the like. In accordance with the present disclosure, any one or any mixture or combination of chelating agents can be contained in a pharmaceutical formulation. The amount of active ingredient that can be combined with the carrier materials to produce a single dosage form will vary depending upon the particular mode of administration.

Within the mucosal delivery compositions and methods of this disclosure, various mucosal delivery-enhancing agents are employed which enhance delivery of NMDP into or across a mucosal surface. In this regard, delivery of NMDP across the mucosal epithelium can occur “transcellularly” or “paracellularly.” The extent to which these pathways contribute to the overall flux and bioavailability of the NMDP depends upon the environment of the mucosa, the physico-chemical properties the active agent, and the properties of the mucosal epithelium. In some embodiments, the methods and compositions of this disclosure provide for significantly enhanced transport of NMDP or a salt thereof into and across mucosal epithelia via the paracellular route. In some embodiments, the methods and compositions of this disclosure provide for significantly enhanced transport of NMDP or a salt thereof into and across mucosal epithelia via the transcellular route. In some embodiments, the methods and compositions of this disclosure provide for significantly enhanced transport of NMDP or a salt thereof into and across mucosal epithelia via the paracellular route, transcellular route, alternatively or within a single method or composition.

As used herein, mucosal delivery-enhancing agents include agents which enhance or otherwise modulate the release or solubility (e.g., from a formulation delivery vehicle), diffusion rate, penetration capacity and timing, uptake, residence time, stability, effective half-life, peak or sustained concentration levels, clearance and other desired mucosal delivery characteristics (e.g., as measured at the site of delivery, or at a selected target site of activity such as the bloodstream or central nervous system) of NMDP or other biologically active compound(s). Enhancement of mucosal delivery can thus occur by any one or more of a variety of mechanisms, for example by increasing the diffusion, transport, persistence or stability of NMDP, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junctional physiology, reducing the viscosity of mucus overlying the mucosal epithelium, reducing mucociliary clearance rates, and other mechanisms.

As used herein, a “mucosally effective amount of NMDP” contemplates effective mucosal delivery of NMDP to a target site (i.e., auric structures) for drug activity in a subject in need thereof that can involve a variety of delivery or transfer routes. For example, a NMDP or a salt thereof can find its way through clearances (e.g., spaces) between cells of the mucosa and reach an adjacent vascular wall in the eustachian tube, while by another route the agent can, either passively or actively, be taken up (i.e., internalized) into mucosal cells to act within the cells or be discharged (e.g., released) or transported out of the cells to reach a secondary target site, such as the tympanic membrane. The methods and compositions of this disclosure can promote the translocation of NMDP or a salt thereof along one or more such alternate (transcellular or paracellular) routes, or can act directly on the mucosal tissue or proximal vascular tissue to promote absorption or penetration of the NMDP or a salt thereof. The promotion of absorption or penetration in this context is not limited to these mechanisms.

Suitable surfactants that can be used in accordance with the present disclosure include ionic, nonionic or amphoteric surface-active agents. More specifically, hydrophilic surfactants (e.g., Tweens, Tween 80, Myrj, Brjs, Labrasol etc.) or lipophilic surfactants (eg. Span 20, Span 60, Myrj, Arlacel 83 and such) can be suitably used, preferably at a concentration in the range of 0-25% by weight.

Suitable preservatives that can be used with the present formulations include, for example, benzyl alcohol, parabens, chlorobutanol, benzalkonium salts and combinations thereof. Some examples of antioxidants include tocopherols, butyl hydroxytoluene, sodium metabisulfite, potassium metabisulfite, ascorbyl palmitate and the like. These preservatives and antioxidants can be present in the formulations in a concentration of from about 0.001% up to about 5% w/w.

Regarding buffers, the nasal delivery system can include a buffer for maintaining the formulation at a pH of about 7.0. The particular buffer, of course, can vary depending upon the particular nasal delivery system used, as well as the specific active molecule selected. Buffers that are suitable for use in the present disclosure include, for example, acetate, citrate, prolamine, carbonate and phosphate buffers and combinations thereof. The pharmaceutical formulations of the present disclosure can include a pH adjusting agent.

Regarding thickening agents, the viscosity of the formulations of the present disclosure can be maintained at a desired level using a pharmaceutically acceptable thickening agent. Thickening agents that can be added to the compositions of the present disclosure include for example, methyl cellulose, xanthan gum, tragacanth, adhesives, guar gum, carboxymethyl cellulose, hydroxypropyl cellulose, carbomer, polyvinyl alcohol, alginates, acacia, chitosans, mucoadhesive polymer-systems like poly(acrylates), cellulose derivatives, hyaluronic acid, hyaluronic acid derivatives, chitin, collagen, pectin, starch, poly(ethylene glycol), sulfated polysaccharides, carrageenan, Na-alginate, gelatin, pectin and combinations thereof. The desired concentration of the thickening agent will depend upon the agent selected and the viscosity desired.

The compositions can also comprise gel forming or bioadhesive compounds such as carbopols, alginates, scleroglucan, cellulose derivatives, starch, albumin, pluronic gels, diethyl aminoethyl (DEAE)-sephadex, polycarbophil, hyaluronic acid, hyaluronates, starch, gelatin, cholagen and others. Compositions can also be incorporated in the w/o cream, o/w cream, hydrophilic ointment or lipophilic ointment, gels, other semi-solid bases. The compositions could be delivered to the nasal cavity as drops, mists, aerosols, instillations, by use of pipetor, special devices, evaporators, vaporizators and such.

The formulations of the present disclosure can also include agents such as tolerance enhancers to reduce or prevent drying of the mucus membrane and to prevent irritation thereof.

In some embodiments, NMDP or a salt thereof or can also be brought into a viscous base by adding to the above delivery systems conventionally used ingredients such as natural gums, cellulose and derivatives, acrylic polymers (eg. carbopol) and vinyl polymers (polyvinylpyrrolidone), scleroglucans, xylan, alginates, calcium alginate, hyaluronates, collagenates, starch gels, gelatin systems, kitosan carriers.

In some embodiments, the compositions disclosed herein can further comprise Benzalkonium chloride (BKC), and/or Ethylenediaminetetraacetic acid (EDTA). In some embodiments, the compositions can comprise, for example, 0.01, 0.05, 01, 0.2, 0.3, or 0.5% w/v of BKC. In some embodiments, the compositions can comprise, 0.01, 0.05, 01, 0.2, 0.3, or 0.5% w/v of EDTA.

While the mechanism of absorption promotion can vary with different mucosal delivery-enhancing agents of this disclosure, useful reagents in this context will not substantially adversely affect the mucosal tissue and will be selected according to the physicochemical characteristics of the particular NMDP or other active or delivery-enhancing agent. In this context, delivery-enhancing agents that increase penetration or permeability of mucosal tissues will often result in some alteration of the protective permeability barrier of the mucosa. For such delivery-enhancing agents to be of value within this disclosure, it is generally desired that any significant changes in permeability of the mucosa can be reversible within a time frame appropriate to the desired duration of drug delivery. Furthermore, there should be no substantial, cumulative toxicity, nor any permanent deleterious changes induced in the barrier properties of the mucosa with long-term use.

Within certain aspects of this disclosure, absorption-promoting agents for coordinate administration or combinatorial formulation with NMDP are selected from small hydrophilic molecules, including but not limited to, dimethyl sulfoxide (DMSO), dimethylformamide, ethanol, propylene glycol, and the 2-pyrrolidones. Alternatively, long-chain amphipathic molecules, for example, deacylmethyl sulfoxide, azone, sodium laurylsulfate, oleic acid, and the bile salts, can be employed to enhance mucosal penetration of the NMDP. In additional aspects, surfactants (e.g., polysorbates) are employed as adjunct compounds, processing agents, or formulation additives to enhance intranasal delivery of the NMDP. Agents such as DMSO, polyethylene glycol, and ethanol can, if present in sufficiently high concentrations in delivery environment (e.g., by pre-administration or incorporation in a therapeutic formulation), enter the aqueous phase of the mucosa and alter its solubilizing properties, thereby enhancing the partitioning of the NMDP from the vehicle (e.g., the therapeutic or pharmaceutical formulation) into the mucosa.

Additional mucosal delivery-enhancing agents that are useful within the coordinate administration and processing methods and combinatorial formulations include, but are not limited to, mixed micelles; enamines; nitric oxide donors (e.g., S-nitroso-N-acetyl-DL-penicillamine, NOR1, NOR4—which are preferably co-administered with an NO scavenger such as carboxy-PITO or doclofenac sodium); sodium salicylate; glycerol esters of acetoacetic acid (e.g., glyceryl-1,3-diacetoacetate or 1,2-isopropylideneglycerine-3-acetoacetate); and other release-diffusion or intra- or trans-epithelial penetration-promoting agents that are physiologically compatible for mucosal delivery.

Other absorption-promoting agents can be selected from a variety of carriers, bases and excipients that enhance mucosal delivery, stability, activity or trans-epithelial penetration of the NMDP. These include, inter alia, cyclodextrins (e.g., cyclodextrin) and β-cyclodextrin derivatives (e.g., hydroxypropyl-β-cyclodextrin, sulfobutylether-β-cyclodextrin, methyl-β-cyclodextrin and heptakis (2,6-di-O-methyl-β-cyclodextrin)). These compounds, optionally conjugated with one or more of the active ingredients and further optionally formulated in an oleaginous base, enhance bioavailability of a glucose regulating peptide contained in the mucosal formulations of this disclosure. Yet additional absorption-enhancing agents adapted for mucosal delivery include medium-chain fatty acids, including mono- and diglycerides (e.g., sodium caprate—extracts of coconut oil, Capmul), and triglycerides (e.g., amylodextrin, Estaram 299, Miglyol 810).

The mucosal therapeutic and prophylactic compositions of the present disclosure can be supplemented with any suitable penetration-promoting agent that facilitates absorption, diffusion, or penetration of NMDP across mucosal barriers. The penetration promoting agent can be any such agent that is pharmaceutically acceptable. Thus, in more detailed aspects of this disclosure compositions are provided that incorporate one or more of the penetration-promoting agents selected from sodium salicylate and salicylic acid derivatives (acetyl salicylate, choline salicylate, salicylamide, etc.); amino acids and salts thereof (e.g., monoaminocarboxlic acids such as glycine, alanine, phenylalanine, proline, hydroxyproline, etc.; hydroxyamino acids such as serine; acidic amino acids such as aspartic acid, glutamic acid, etc.; and basic amino acids such as lysine etc. —inclusive of their alkali metal or alkaline earth metal salts); and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts). Also provided as penetration-promoting agents within the methods and compositions of this disclosure are substances which are generally used as emulsifiers (e.g., sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidonecarboxylic acid esters, N-alkylpyrrolidones, proline acyl esters, and the like.

A range of components and additives are contemplated for use within the methods and formulations of the present disclosure. Exemplary of such solubilization agents are cyclodextrins (CDs) and derivatives thereof. These CDs have been found to bind to hydrophobic patches of proteins in a manner that significantly inhibits aggregation. This inhibition is selective with respect to both the CD and the protein involved. Such selective inhibition of protein aggregation provides additional advantages within the intranasal delivery methods and compositions of the disclosure. Additional agents for use in this context include CD dimers, trimers and tetramers with varying geometries controlled by linkers, peptides, peptide derivatives, analogues, and peptide mimetics to selectively block protein-protein interactions. In one aspect, the specific binding of hydrophobic side chains reported for CD multimers is extended to proteins via the use of peptides and peptide mimetics that similarly block protein aggregation. A wide range of suitable methods and anti-aggregation agents are available for incorporation within the compositions and procedures contemplated herein.

### Charge Modifying and pH Control Agents and Methods

To improve the transport characteristics of biologically active agents (including a NMDP), or other active macromolecular and small molecule drugs for enhanced delivery across hydrophobic mucosal membrane barriers, the compositions herein can also use reagents for the “charge modification” of selected biologically active agents or delivery-enhancing agents described herein. In this regard, the relative permeability of a macromolecule is generally related to its partition coefficient. A molecules degree of ionization, which is dependent on the pKa of the molecule and the pH at the mucosal membrane surface, also affects its permeability. As set forth herein, the permeation and partitioning of biologically active agents, including NMDP and analogs thereof, for mucosal delivery can be facilitated by charge alteration or charge spreading of the active agent or permeabilizing agent, which can be achieved, for example, by alteration of charged functional groups, by modifying the pH of the delivery vehicle or solution in which the active agent (or precursor thereto) is delivered, or by coordinate administration of a charge- or pH-altering reagent with the active agent (or precursor).

Consistent with these general teachings, mucosal delivery of charged macromolecular species, including NMDP and other biologically active peptides and proteins, within the methods and compositions of this disclosure is substantially improved when the active agent is delivered to the mucosal surface in a substantially un-ionized, or neutral, electrical charge state.

Certain compositions comprising NMDP salts (NMDP) (s) and other biologically active peptide and protein components of one or more mucosal formulations within this disclosure can be charge modified in order to provide an increase in the positive charge density of the peptide or protein. These modifications extend also to cationization of peptide and protein conjugates, carriers and other delivery forms disclosed herein. Cationization offers a convenient means of altering the biodistribution and transport properties of proteins and macromolecules within this disclosure. Cationization is undertaken in a manner that substantially preserves the biological activity of the active agent and limits potentially adverse side effects, including tissue damage and toxicity.

In some embodiments, compositions can comprise a buffer solution. A “buffer” is generally used to maintain the pH of a solution at a nearly constant value. Examples of commonly used buffer salts include the following: glutamate, acetate, citrate, glycine, histidine, arginine, lysine, methionine, lactate, formate, glycolate, tartrate, phosphate and mixtures thereof.

### Mucolytic and Mucus-Clearing Agents and Methods

In some embodiments, the compositions and methods herein for the mucosal delivery, for example, intranasal delivery of NMDP or a salt thereof, can optionally incorporate effective mucolytic or mucus-clearing agents, which serve to degrade, thin or clear mucus from intranasal mucosal surfaces to facilitate absorption of the intranasally administered composition. In some embodiments, a mucolytic or mucus-clearing agent is coordinately administered as an adjunct compound to enhance intranasal delivery of the biologically active agent. Alternatively, an effective amount of a mucolytic or mucus-clearing agent is incorporated as a processing agent within a multi-processing method of this disclosure, or as an additive within a combinatorial formulation of this disclosure, to provide an improved formulation that enhances intranasal delivery of biotherapeutic compounds by reducing the barrier effects of intranasal mucus.

A variety of mucolytic or mucus-clearing agents are available for incorporation within the methods and compositions of this disclosure. Based on their mechanisms of action, mucolytic and mucus clearing agents can often be classified into the following groups: proteases (e.g., pronase, papain) that cleave the protein core of mucin glycoproteins; sulfhydryl compounds that split mucoprotein disulfide linkages; and detergents (e.g., Triton X-100, Tween 20) that break non-covalent bonds within the mucus. Additional compounds in this context include, but are not limited to, bile salts and surfactants, for example, sodium deoxycholate, sodium taurodeoxycholate, sodium glycocholate, and lysophosphatidylcholine.

The effectiveness of bile salts in causing structural breakdown of mucus is in the order deoxycholate>taurocholate>glycocholate. Other effective agents that reduce mucus viscosity or adhesion to enhance intranasal delivery according to the methods of this disclosure include, e.g., short-chain fatty acids, and mucolytic agents that work by chelation, such as N-acylcollagen peptides, bile acids, and saponins (the latter function in part by chelating Ca²⁺ and/or Mg²⁺ which play an important role in maintaining mucus layer structure).

Additional mucolytic agents for use within the methods and compositions of this disclosure include N-acetyl-L-cysteine (ACS), a potent mucolytic agent that reduces both the viscosity and adherence of bronchopulmonary mucus and is reported to modestly increase nasal bioavailability of human growth hormone in anesthetized rats (from 7.5 to 12.2%). These and other mucolytic or mucus-clearing agents are contacted with the nasal mucosa, typically in a concentration range from about 0.2 to about 20 mM, coordinately with administration of the biologically active agent, to reduce the polar viscosity and/or elasticity of intranasal mucus.

Still other mucolytic or mucus-clearing agents can be selected from a range of glycosidase enzymes, which are able to cleave glycosidic bonds within the mucus glycoprotein. α-amylase and β-amylase are representative of this class of enzymes, although their mucolytic effect can be limited. In contrast, bacterial glycosidases allow these microorganisms to permeate mucus layers of their hosts.

For combinatorial use with the biologically active agents described within this disclosure, non-ionogenic detergents are generally also useful as mucolytic or mucus-clearing agents.

### Ciliostatic Agents and Methods

Because the self-cleaning capacity of certain mucosal tissues (e.g., nasal mucosal tissues) by mucociliary clearance is necessary as a protective function (e.g., to remove dust, allergens, and bacteria), it is appreciated that this function should not be substantially impaired by mucosally administered medications. Mucociliary transport in the respiratory tract is a particularly important defense mechanism against infections. To achieve this function, ciliary beating in the nasal and airway passages moves a layer of mucus along the mucosa to removing inhaled particles and microorganisms.

In some embodiments, ciliostatic agents can be incorporated within the methods and compositions of this disclosure to increase the residence time of a mucosally (e.g., intranasally) administered formulation comprising NMDP, and other biologically active agent disclosed herein. In particular, the delivery of such agents within the methods and compositions of this disclosure is significantly enhanced in certain aspects by the coordinate administration or combinatorial formulation of one or more ciliostatic agents that function to reversibly inhibit the ciliary activity of mucosal cells, and thereby to provide for a temporary, reversible increase in the residence time of the mucosally administered pharmaceutically active agent(s). For use within these aspects of this disclosure, the ciliostatic factors set forth herein, either specific or indirect in their activity, are all candidates for successful employment as a ciliostatic agent in appropriate amounts (depending on concentration, duration and mode of delivery) such that they yield a transient (i.e., reversible) reduction or cessation of mucociliary clearance at a mucosal site of administration to enhance delivery of NMDP, and other biologically active agents disclosed herein, without unacceptable adverse side effects.

Within more detailed aspects, a specific ciliostatic factor can be employed in a combined formulation or coordinate administration protocol with NMDP and a salt thereof and one or more other biologically active agent disclosed herein. Various bacterial ciliostatic factors isolated and characterized in the literature can be employed within certain embodiments of this disclosure. For example, ciliostatic factors from the bacterium Pseudomonas aeruginosa include a phenazine derivative, a pyo compound (2-alkyl-4-hydroxyquinolines), and a rhamnolipid (also known as a hemolysin). In some embodiments, phenazine derivative can also inhibit ciliary motility. In some embodiments, compositions can comprise rhamnolipid, which is associated with altered ciliary membranes.

### Surface Active Agents and Methods

Within more detailed aspects of this disclosure, one or more membrane penetration-enhancing agents can be employed within a mucosal delivery method or formulation of this disclosure to enhance mucosal delivery of NMDP, and other biologically active agents disclosed herein. Membrane penetration enhancing agents in this context can be selected from: (i) a surfactant; (ii) a bile salt; (iii) a phospholipid additive, mixed micelle, liposome, or carrier; (iv) an alcohol; (v) an enamine; (vi) an NO donor compound; (vii) a long-chain amphipathic molecule; (viii) a small hydrophobic penetration enhancer; (ix) sodium or a salicylic acid derivative; (x) a glycerol ester of acetoacetic acid; (xi) a cyclodextrin or beta-cyclodextrin derivative; (xii) a medium-chain fatty acid; (xiii) a chelating agent; (xiv) an amino acid or salt thereof; (xv) an N-acetylamino acid or salt thereof; (xvi) an enzyme degradative to a selected membrane component; (xvii) an inhibitor of fatty acid synthesis; (xviii) an inhibitor of cholesterol synthesis; or (xix) any combination of the membrane penetration enhancing agents recited in (i)-(xviii).

Certain surface-active agents, also called surfactants, are readily incorporated within the mucosal delivery formulations and methods of this disclosure as mucosal absorption enhancing agents. These agents, which can be coordinately administered or combinatorically formulated with NMDP, and other biologically active agents disclosed herein, can be selected from a broad assemblage of known surfactants. Surfactants, which generally fall into three classes: (1) nonionic polyoxyethylene ethers, such as Vitamin E TPGS, and/or D-α-tocopheryl polyethylene glycol succinate; (2) bile salts such as sodium glycocholate (SGC) and deoxycholate (DOC); and (3) fusidic acid and derivatives of fusidic acid such as sodium taurodihydrofusidate (STDHF). The mechanisms of action of these various classes of surface-active agents typically include solubilization of the biologically active agent. For proteins and peptides which often form aggregates, the surface active properties of these absorption promoters can allow interactions with proteins such that smaller units such as surfactant coated monomers can be more readily maintained in solution. These monomers are presumably more transportable units than aggregates. Examples of other surface-active agents are L-α-Phosphatidylcholine Didecanoyl (DDPC), polysorbate 80 and polysorbate 20. Additional surface-acting agents include polyethylene glycol, cetyl alcohol, polyvinylpyrolidone, polyvinyl alcohol, lanolin alcohol, sorbitan monooleate. In some embodiments, surface-acting agents of the instant disclosure can be present in a pharmaceutical formulation alone or in any mixture or combination. In some embodiments, bile salts and some fusidic acid derivatives reportedly inhibit proteolytic degradation of proteins by nasal homogenates.

### Thickening Agents

Thickening or suspending agents can affect the rate of release of a drug from the dosage formulation and/or absorption. Some examples of the materials which can serve as pharmaceutically acceptable thickening agents are gelatin; methylcellulose (MC); hydroxypropylmethylcellulose (HPMC) and derivatives thereof; carboxymethylcellulose (CMC); cellulose; starch; heta starch; poloxamers; pluronics; sodium CMC; sorbitol; acacia; povidone; carbopol (as used herein, carbopol is a carbomer; carbopol is also known as Carbomer Homopolymer Type B, or Carbopol® 974P NF Polymer); polycarbophil; chitosan; chitosan microspheres; alginate microspheres; chitosan glutamate; amberlite resin; hyaluronan; ethyl cellulose; maltodextrin DE; drum-dried way maize starch (DDWM); degradable starch microspheres (DSM); deoxyglycocholate (GDC); hydroxyethyl cellulose (HEC); hydroxypropyl cellulose (HPC); microcrystalline cellulose (MCC); polymethacrylic acid and polyethylene glycol; sulfobutylether B cyclodextrin; cross-linked eldexomer starch biospheres; sodiumtaurodihydrofusidate (STDHF); N-trimethyl chitosan chloride (TMC); degraded starch microspheres; amberlite resin; chistosan nanoparticles; spray-dried crospovidone; spray-dried dextran microspheres; spray-dried microcrystalline cellulose; and cross-linked eldexomer starch microspheres.

As used herein, a carbomer thickening agent also includes, but is not limited to, the following: Acrylic acid homopolymer, Acrylic acid resin, Acrylic acid, polymer, Acrylic polymer, Acrylic resin, Acrysol A 1, Acrysol A 3, Acrysol A 5, Acrysol AC 5, Acrysol WS-24, Acrysol ase-75, Antiprex 461, Antiprex A, Arasorb 750, Arasorb S100F, Arolon, Aron, Aron A 10H, Atactic poly(acrylic acid), CCRIS 3234, Carbomer 1342, Carbomer 910, Carbopol 1342, Carbopol 910, Carbopol 934, Carbopol 934P, Carbopol 940, Carbopol 941, Carbopol 960, Carbopol 961, Carbopol 971P, Carbopol 974P, Carbopol 980, Carbopol 981, Carboset 515, Carboset Resin No. 515, Carboxy vinyl polymer, Carboxypolymethylene, Carpolene, Colloids 119/50, Cyguard 266, Dispex C40, Dow Latex 354, G-Cure, Good-rite K 37, Good-rite K 702, Good-rite K 732, Good-rite K-700, Good-rite K727, Good-rite WS 801, Haloflex 202, Haloflex 208, Joncryl 678, Junlon 110, Jurimer AC 10H, Jurimer AC 10P, NSC 106034, NSC 106035, NSC 106036, NSC 106037, NSC 112122, NSC 112123, NSC 114472, NSC 165257, Nalfloc 636, Neocryl A-1038, OLD 01, P 11H, P 11H, P-11H, PA 11M, PAA-25, Pemulen TR-1, Pemulen TR-2, Poly(acrylic acid), Polyacrylate, Polyacrylate elastomers, Polymer of acrylic acid, cross-linked with allyl ethers of pentaerythritol, Polymer of acrylic acid, cross-linked with allyl ethers of pentaerythritol, carboxy vinyl Polymerized acrylic acid, Polytex 973, Primal ASE 60, Propenoic acid polymer, R968, Racryl, Revacryl A 191, Rohagit SD 15, Sokalan PAS, Solidokoll N, Synthemul 90-588, TB 1131, Tecpol, Texcryl, Versicol E 7, Versicol E15, Versicol E9, Versicol K 11, Versicol S 25, Viscalex HV 30, Viscon 103, WS 24, WS 801, XPA and the like. Other thickening agents in Ugwoke et al., Adv. Drug Deliv. Rev. 29:1656-57, 1998, are incorporated by reference. Any one thickening agent or any combination or mixture of thickening increasing agents can be contained in a pharmaceutical formulation disclosed herein.

### Nitric Oxide Donor Agents and Methods

Within other related aspects of this disclosure, a nitric oxide (NO) donor is selected as a membrane penetration-enhancing agent to enhance mucosal delivery of one or more NMDP, and other biologically active agents disclosed herein. Various NO donors are known in the art and are useful in effective concentrations within the methods and formulations of this disclosure. Exemplary NO donors include, but are not limited to, nitroglycerine, nitropruside, NOC5 [3-(2-hydroxy-1-(methyl-ethyl)-2-nitrosohydrazino)-1-propanamine], NOC12 [N-ethyl-2-(1-ethyl-hydroxy-2-nitrosohydrazino)-ethanamine], SNAP [S-nitroso-N-acetyl-DL-penicillamine], NORI and NOR4. Within the methods and compositions of this disclosure, an effective amount of a selected NO donor is coordinately administered or combinatorically formulated with one or more NMDP, and/or other biologically active agents disclosed herein, into or through the mucosal epithelium.

Agents for Modulating Epithelial Junction Structure and/or Physiology

The present disclosure provides pharmaceutical compositions that contain one or more NMDP and/or other biologically active agents in combination with one or more mucosal delivery enhancing agent disclosed herein formulated in such pharmaceutical preparation for mucosal delivery.

The permeabilizing agent reversibly enhances mucosal epithelial paracellular transport, typically by modulating epithelial junctional structure and/or physiology at a mucosal epithelial surface in the subject. This effect typically involves inhibition by the permeabilizing agent of homotypic or heterotypic binding between epithelial membrane adhesive proteins of neighboring epithelial cells. Target proteins for this blockade of homotypic or heterotypic binding can be selected from various related junctional adhesion molecules (JAMs), occludins, or claudins. Examples of this are antibodies, antibody fragments or single-chain antibodies that bind to the extracellular domains of these proteins.

In yet additional detailed embodiments, this disclosure provides permeabilizing peptides for enhancing mucosal epithelial paracellular transport. The peptides typically work within the compositions and methods of this disclosure by modulating epithelial junctional structure and/or physiology in a mammalian subject. In certain embodiments, the peptides inhibit homotypic and/or heterotypic binding of an epithelial membrane adhesive protein selected from a junctional adhesion molecule (JAM), occludin, or claudin.

One such agent that has been extensively studied is the bacterial toxin from Vibrio cholerae known as the “zonula occludens toxin” (ZOT). Within these aspects of this disclosure, ZOT is coordinately administered or combinatorially formulated with the active agents in the compositions and methods herein, in an effective amount to yield significantly enhanced absorption of the active agent, by reversibly increasing nasal mucosal permeability without substantial adverse side effects.

### Vasodilator Agents and Methods

In some embodiments, the compositions and methods herein can include administration of vasoactive compounds, more specifically vasodilators. These compounds function within the present disclosure to modulate the structure and physiology of the submucosal vasculature, increasing the transport rate of NMDP, and other biologically active agents into or through the mucosal epithelium and/or to specific target tissues or compartments (e.g., auric structures).

Vasodilator agents for use within this disclosure typically cause submucosal blood vessel relaxation by either a decrease in cytoplasmic calcium, an increase in nitric oxide (NO) or by inhibiting myosin light chain kinase. They are generally divided into 9 classes: calcium antagonists, potassium channel openers, ACE inhibitors, angiotensin-II receptor antagonists, a-adrenergic and imidazole receptor antagonists, β1-adrenergic agonists, phosphodiesterase inhibitors, eicosanoids and NO donors.

Within certain methods and compositions of this disclosure, a selected vasodilator agent is coordinately administered (e.g., systemically or intranasally, simultaneously or in combinatorially effective temporal association) or combinatorially formulated with one or more NMDP in an amount effective to enhance the mucosal absorption of the active agent(s) to reach a target tissue or compartment in the subject (e.g., the auric structures).

### Selective Transport-Enhancing Agents and Methods

The compositions and delivery methods of this disclosure optionally incorporate a selective transport-enhancing agent that facilitates transport of one or more biologically active agents. These transport-enhancing agents can be employed in a combinatorial formulation or coordinate administration protocol with one or more of the NMDP formulations disclosed herein to coordinately enhance delivery of one or more additional biologically active agent(s) across mucosal transport barriers, to enhance mucosal delivery of the active agent(s) to reach a target tissue or compartment in the subject (e.g., the auric structures). Alternatively, the transport-enhancing agents can be employed in a combinatorial formulation or coordinate administration protocol to directly enhance mucosal delivery of one or more of the NMDP, with or without enhanced delivery of an additional biologically active agent.

Exemplary selective transport-enhancing agents for use within this aspect of this disclosure include, but are not limited to, glycosides, sugar-containing molecules, and binding agents such as lectin binding agents, which are known to interact specifically with epithelial transport barrier components. For example, specific “bioadhesive” ligands, including various plant and bacterial lectins, which bind to cell surface sugar moieties by receptor-mediated interactions can be employed as carriers or conjugated transport mediators for enhancing mucosal, e.g., nasal delivery of biologically active agents within this disclosure. Certain bioadhesive ligands within this disclosure will mediate transmission of biological signals to epithelial target cells that trigger selective uptake of the adhesive ligand by specialized cellular transport processes (endocytosis or transcytosis). These transport mediators can therefore be employed as a “carrier system” to stimulate or direct selective uptake of one or more NMDP, and other biologically active agent(s) into and/or through mucosal epithelia. These and other selective transport-enhancing agents significantly enhance mucosal delivery of macromolecular biopharmaceuticals (particularly peptides, proteins, oligonucleotides and polynucleotide vectors) within this disclosure. Lectins are plant proteins that bind to specific sugars found on the surface of glycoproteins and glycolipids of eukaryotic cells. Concentrated solutions of lectins have a ‘mucotractive’ effect, and various studies have demonstrated rapid receptor mediated endocytocis (RME) of lectins and lectin conjugates (e.g., concanavalin A conjugated with colloidal gold particles) across mucosal surfaces. Additional studies have reported that the uptake mechanisms for lectins can be utilized for intestinal drug targeting in vivo. In certain of these studies, polystyrene nanoparticles (500 nm) were covalently coupled to tomato lectin and reported yielded improved systemic uptake after oral administration to rats.

In addition to plant lectins, microbial adhesion and invasion factors provide a rich source of candidates for use as adhesive/selective transport carriers within the mucosal delivery methods and compositions of this disclosure. Two components are necessary for bacterial adherence processes, a bacterial ‘adhesin’ (adherence or colonization factor) and a receptor on the host cell surface. Bacteria causing mucosal infections need to penetrate the mucus layer before attaching themselves to the epithelial surface. This attachment is usually mediated by bacterial fimbriae or pilus structures, although other cell surface components can also take part in the process. Adherent bacteria colonize mucosal epithelia by multiplication and initiation of a series of biochemical reactions inside the target cell through signal transduction mechanisms (with or without the help of toxins). Associated with these invasive mechanisms, a wide diversity of bioadhesive proteins (e.g., invasin, internalin) originally produced by various bacteria and viruses are known. These allow for extracellular attachment of such microorganisms with an impressive selectivity for host species and even particular target tissues. Signals transmitted by such receptor-ligand interactions trigger the transport of intact, living microorganisms into, and eventually through, epithelial cells by endo- and transcytotic processes. Such naturally occurring phenomena can be harnessed (e.g., by complexing biologically active agents such as NMDP with an adhesin) according to the teachings herein for enhanced delivery of biologically active compounds into or across mucosal epithelia and/or to other designated target sites of drug action.

Various bacterial and plant toxins that bind epithelial surfaces in a specific, lectin-like manner are also useful within the methods and compositions of this disclosure. For example, diphtheria toxin (DT) enters host cells rapidly by RME. Likewise, the B subunit of the E. coli heat labile toxin binds to the brush border of intestinal epithelial cells in a highly specific, lectin-like manner. Uptake of this toxin and transcytosis to the basolateral side of the enterocytes has been reported in vivo and in vitro. Other researches have expressed the transmembrane domain of diphtheria toxin in E. coli as a maltose-binding fusion protein and coupled it chemically to high-Mw poly-L-lysine. The resulting complex is successfully used to mediate internalization of a reporter gene in vitro. In addition to these examples, Staphylococcus aureus produces a set of proteins (e.g., staphylococcal enterotoxin A (SEA), SEB, toxic shock syndrome toxin 1 (TSST-1) which act both as superantigens and toxins. Studies relating to these proteins have reported dose-dependent, facilitated transcytosis of SEB and TSST-1 in Caco-2 cells.

Viral haemagglutinins comprise another type of transport agent to facilitate mucosal delivery of biologically active agents within the methods and compositions of this disclosure. The initial step in many viral infections is the binding of surface proteins (haemagglutinins) to mucosal cells. These binding proteins have been identified for most viruses, including rotaviruses, varicella zoster virus, semliki forest virus, adenoviruses, potato leafroll virus, and reovirus. These and other exemplary viral hemagglutinins can be employed in a combinatorial formulation (e.g., a mixture or conjugate formulation) or coordinate administration protocol with one or more of the NMDP, disclosed herein, to coordinately enhance mucosal delivery of one or more additional biologically active agent(s). Alternatively, viral hemagglutinins can be employed in a combinatorial formulation or coordinate administration protocol to directly enhance mucosal delivery of one or more of the NMDP, with or without enhanced delivery of an additional biologically active agent.

A variety of endogenous, selective transport-mediating factors are also available for use within this disclosure. Mammalian cells have developed an assortment of mechanisms to facilitate the internalization of specific substrates and target these to defined compartments. Collectively, these processes of membrane deformations are termed ‘endocytosis’ and comprise phagocytosis, pinocytosis, receptor-mediated endocytosis (clathrin-mediated RME), and potocytosis (non-clathrin-mediated RME). RME is a highly specific cellular biologic process by which, as its name implies, various ligands bind to cell surface receptors and are subsequently internalized and trafficked within the cell. In many cells the process of endocytosis is so active that the entire membrane surface is internalized and replaced in less than a half hour. Two classes of receptors are proposed based on their orientation in the cell membrane; the amino terminus of Type I receptors is located on the extracellular side of the membrane, whereas Type II receptors have this same protein tail in the intracellular milieu.

Still other embodiments of this disclosure utilize transferrin as a carrier or stimulant of RME of mucosally delivered biologically active agents. Transferrin, an 80 kDa iron-transporting glycoprotein, is efficiently taken up into cells by RME. Transferrin receptors are found on the surface of most proliferating cells, in elevated numbers on erythroblasts and on many kinds of tumors. The transcytosis of transferrin (Tf) and transferrin conjugates is reportedly enhanced in the presence of Brefeldin A (BFA), a fungal metabolite. In other studies, BFA treatment has been reported to rapidly increase apical endocytosis of both ricin and HRP in MDCK cells. Thus, BFA and other agents that stimulate receptor-mediated transport can be employed within the methods of this disclosure as combinatorially formulated (e.g., conjugated) and/or coordinately administered agents to enhance receptor-mediated transport of biologically active agents, for example, NMDP.

### Polymeric Delivery Vehicles and Methods

Within certain aspects of the disclosure, NMDP, other biologically active agents disclosed herein, and delivery-enhancing agents as described herein, are, individually or combinatorically, incorporated within a mucosally (e.g., nasally) administered formulation that includes a biocompatible polymer functioning as a carrier or base. Such polymer carriers include polymeric powders, matrices or microparticulate delivery vehicles, among other polymer forms. The polymer can be of plant, animal, or synthetic origin. Often the polymer is crosslinked. Additionally, in these delivery systems the NMDP, can be functionalized in a manner where it can be covalently bound to the polymer and rendered inseparable from the polymer. In other embodiments, the polymer is chemically modified with an inhibitor of enzymes or other agents which can degrade or inactivate the biologically active agent(s) and/or delivery enhancing agent(s). In certain formulations, the polymer is a partially or completely water insoluble but water swellable polymer, e.g., a hydrogel. In some embodiments, the polymer is water interactive and/or hydrophilic in nature.

For prolonging the biological activity of NMDP, and other biologically active agents disclosed herein, as well as optional delivery-enhancing agents, these agents can be incorporated into polymeric matrices, e.g., polyorthoesters, polyanhydrides, or polyesters. In some embodiments, this yields sustained activity and release of the active agent(s), e.g., as determined by the degradation of the polymer matrix. Absorption-promoting polymers contemplated for use within this disclosure can include derivatives and chemically or physically modified versions of the foregoing types of polymers, in addition to other naturally occurring or synthetic polymers, gums, resins, and other agents, as well as blends of these materials with each other or other polymers. In some aspects of the formulations in this disclosure, polymers such as nylon, acrylan and other normally hydrophobic synthetic polymers can be sufficiently modified by reaction to become water swellable and/or form stable gels in aqueous media. Absorption-promoting polymers of this disclosure can include polymers from the group of homo- and copolymers based on various combinations of the following vinyl monomers: acrylic and methacrylic acids, acrylamide, methacrylamide, hydroxyethylacrylate or methacrylate, vinylpyrrolidones, as well as polyvinylalcohol and its co- and terpolymers, polyvinylacetate, its co- and terpolymers with the above listed monomers and 2-acrylamido-2-methyl-propanesulfonic acid (AMPS®). In some embodiments, copolymers of the above listed monomers with copolymerizable functional monomers such as acryl or methacryl amide acrylate or methacrylate esters can be used. Additional absorption-promoting polymers contemplated within this disclosure are those classified as dextrans, dextrins, and from the class of materials classified as natural gums and resins, or from the class of natural polymers such as processed collagen, chitin, chitosan, pullalan, zooglan, alginates and modified alginates such as “Kelcoloid” (a polypropylene glycol modified alginate) gellan gums such as “Kelocogel,” Xanathan gums such as “Keltrol,” estastin, alpha hydroxy butyrate and its copolymers, hyaluronic acid and its derivatives, polylactic and glycolic acids.

In some embodiments, polymers contemplated within the instant disclosure are olefinically-unsaturated carboxylic acids containing at least one activated carbon-to-carbon olefinic double bond, and at least one carboxyl group; that is, an acid or functional group readily converted to an acid containing an olefinic double bond which readily functions in polymerization because of its presence in the monomer molecule, either in the alpha-beta position with respect to a carboxyl group, or as part of a terminal methylene grouping. Olefinically-unsaturated acids of this class include such materials as the acrylic acids typified by the acrylic acid itself, alpha-cyano acrylic acid, beta methylacrylic acid (crotonic acid), alpha-phenyl acrylic acid, beta-acryloxy propionic acid, cinnamic acid, p-chloro cinnamic acid, 1-carboxy-4-phenyl butadiene-1,3, itaconic acid, citraconic acid, mesaconic acid, glutaconic acid, aconitic acid, maleic acid, fumaric acid, and tricarboxy ethylene.

In some embodiments contemplated in the compositions and methods herein, absorption-promoting agents can include methyl acrylate, ethyl acrylate, propyl acrylate, isopropyl acrylate, butyl acrylate, isobutyl acrylate, methyl methacrylate, methyl ethacrylate, ethyl methacrylate, octyl acrylate, heptyl acrylate, octyl methacrylate, isopropyl methacrylate, 2-ethylhexyl methacrylate, nonyl acrylate, hexyl acrylate, n-hexyl methacrylate, and the like. Higher alkyl acrylic esters are decyl acrylate, isodecyl methacrylate, lauryl acrylate, stearyl acrylate, behenyl acrylate and melissyl acrylate and methacrylate versions thereof. Mixtures of two or three or more long chain acrylic esters can be successfully polymerized with one of the carboxylic monomers. Other comonomers include olefins, including alpha olefins, vinyl ethers, vinyl esters, and mixtures thereof.

Other vinylidene monomers, including the acrylic nitriles, can also be used as absorption-promoting agents within the methods and compositions of this disclosure to enhance delivery and absorption of one or more NMDP, and other biologically active agent(s) herein, including to enhance delivery of the active agent(s) to a target tissue or compartment in the subject (e.g., auric structures). In some embodiments, alpha, beta-olefinically unsaturated nitriles can be onoolefinically unsaturated nitriles having from 3 to 10 carbon atoms such as acrylonitrile, methacrylonitrile, and the like. Acrylic amides containing from 3 to 35 carbon atoms including monoolefinically unsaturated amides also can be used. Representative amides include acrylamide, methacrylamide, N-t-butyl acrylamide, N-cyclohexyl acrylamide, higher alkyl amides, where the alkyl group on the nitrogen contains from 8 to 32 carbon atoms, acrylic amides including N-alkylol amides of alpha, beta-olefinically unsaturated carboxylic acids including those having from 4 to 10 carbon atoms such as N-methylol acrylamide, N-propanol acrylamide, N-methylol methacrylamide, N-methylol maleimide, N-methylol maleamic acid esters, N-methylol-p-vinyl benzamide, and the like. In other embodiments, absorption promoting materials are alpha-olefins containing from 2 to 18 carbon atoms, more preferably from 2 to 8 carbon atoms; dienes containing from 4 to 10 carbon atoms; vinyl esters and allyl esters such as vinyl acetate; vinyl aromatics such as styrene, methyl styrene and chloro-styrene; vinyl and allyl ethers and ketones such as vinyl methyl ether and methyl vinyl ketone; chloroacrylates; cyanoalkyl acrylates such as alpha-cyanomethyl acrylate, and the alpha-, beta-, and gamma-cyanopropyl acrylates; alkoxyacrylates such as methoxy ethyl acrylate; haloacrylates as chloroethyl acrylate; vinyl halides and vinyl chloride, vinylidene chloride and the like; divinyls, diacrylates and other polyfunctional monomers such as divinyl ether, diethylene glycol diacrylate, ethylene glycol dimethacrylate, methylene-bis-acrylamide, allylpentaerythritol, and the like; and bis(beta-haloalkyl)alkenyl phosphonates such as bis(beta-chloroethyl) vinyl phosphonate and the like as are known to those skilled in the art. Copolymers wherein the carboxy containing monomer is a minor constituent, and the other vinylidene monomers present as major components are readily prepared in accordance with the methods disclosed herein.

In some embodiments, the absorption promoting agents contemplated in the methods and compositions herein can be composed of synthetic copolymers from the group of acrylic and methacrylic acids, acrylamide, methacrylamide, hydroxyethylacrylate (HEA) or methacrylate (HEMA), and vinylpyrrolidones which are water interactive and swellable. Other very useful hydrogel polymers are swellable, but insoluble versions of poly(vinyl pyrrolidone) starch, carboxymethyl cellulose and polyvinyl alcohol. In some embodiments, polymeric hydrogel materials contemplated within this disclosure include (poly) hydroxyalkyl (meth)acrylate: anionic and cationic hydrogels: poly (electrolyte) complexes; poly(vinyl alcohols) having a low acetate residual: a swellable mixture of crosslinked agar and crosslinked carboxymethyl cellulose: a swellable composition comprising methyl cellulose mixed with a sparingly crosslinked agar; a water swellable copolymer produced by a dispersion of finely divided copolymer of maleic anhydride with styrene, ethylene, propylene, or isobutylene; a water swellable polymer of N-vinyl lactams; swellable sodium salts of carboxymethyl cellulose; and the like.

In some embodiments, polymers contemplated herein for mucosal delivery of biologically active agents, including NMDP, include pectin; polysaccharides such as agar, acacia, karaya, tragacenth, algins and guar and their crosslinked versions; acrylic acid polymers, copolymers and salt derivatives, polyacrylamides; water swellable indene maleic anhydride polymers; starch graft copolymers; acrylate type polymers and copolymers with water absorbability of about 2 to 400 times its original weight; diesters of polyglucan; a mixture of crosslinked poly(vinyl alcohol) and poly(N-vinyl-2-pyrrolidone); polyoxybutylene-polyethylene block copolymer gels; carob gum; polyester gels; poly urea gels; polyether gels; polyamide gels; polyimide gels; polypeptide gels; polyamino acid gels; poly cellulosic gels; crosslinked indene-maleic anhydride acrylate polymers; and polysaccharides. In some embodiments, the hydrogel polymers contemplated in this disclosure are cross-linked to contain effectively the biologically active agent(s).

A crosslinked network can be formed by free radical copolymerization of unsaturated monomers. Polymeric hydrogels can also be formed by cross-linking preformed polymers by reacting functional groups found on the polymers such as alcohols, acids, amines with such groups as glyoxal, formaldehyde or glutaraldehyde, bis anhydrides and the like. The polymers also can be cross-linked with any polyene, e.g., decadiene or trivinyl cyclohexane; acrylamides, such as N,N-methylene-bis(acrylamide); polyfunctional acrylates, such as trimethylol propane triacrylate; or polyfunctional vinylidene monomer containing at least 2 terminal CH2<groups, including, for example, divinyl benzene, divinyl naphthlene, allyl acrylates and the like. In certain embodiments, cross-linking monomers for use in preparing the copolymers are polyalkenyl polyethers having more than one alkenyl ether grouping per molecule, which can optionally possess alkenyl groups in which an olefinic double bond is present attached to a terminal methylene grouping (e.g., made by the etherification of a polyhydric alcohol containing at least 2 carbon atoms and at least 2 hydroxyl groups). Other cross-linking monomers include for example, diallyl esters, dimethallyl ethers, allyl or methallyl acrylates and acrylamides, tetravinyl silane, polyalkenyl methanes, diacrylates, and dimethacrylates, divinyl compounds such as divinyl benzene, polyallyl phosphate, diallyloxy compounds and phosphite esters and the like.

In other aspects of this disclosure, mucosal delivery of NMDP, and other biologically active agents disclosed herein, is enhanced by retaining the active agent(s) in a slow-release or enzymatically or physiologically protective carrier or vehicle, for example a hydrogel that shields the active agent from the action of the degradative enzymes. In certain embodiments, the active agent is bound by chemical means to the carrier or vehicle, to which can also be admixed or bound additional agents such as enzyme inhibitors, cytokines, etc. The active agent can alternately be immobilized through sufficient physical entrapment within the carrier or vehicle, e.g., a polymer matrix.

Polymers such as hydrogels within this disclosure can incorporate functional linked agents such as glycosides chemically incorporated into the polymer for enhancing intranasal bioavailability of active agents formulated therewith. Examples of such glycosides are glucosides, fructosides, galactosides, arabinosides, mannosides and their alkyl substituted derivatives and natural glycosides such as arbutin, phlorizin, amygdalin, digitonin, saponin, and indican. There are several ways in which a typical glycoside can be bound to a polymer. For example, the hydrogen of the hydroxyl groups of a glycoside or other similar carbohydrate can be replaced by the alkyl group from a hydrogel polymer to form an ether. Also, the hydroxyl groups of the glycosides can be reacted to esterify the carboxyl groups of a polymeric hydrogel to form polymeric esters in situ. Another approach is to employ condensation of acetobromoglucose with cholest-5-en-3beta-ol on a copolymer of maleic acid. N-substituted polyacrylamides can be synthesized by the reaction of activated polymers with omega-aminoalkylglycosides: (1) (carbohydrate-spacer)(n)-polyacrylamide, ‘pseudopolysaccharides’; (2) (carbohydrate spacer)(n)-phosphatidylethanolamine(m)-polyacrylamide, neoglycolipids, derivatives of phosphatidylethanolamine; and (3) (carbohydrate-spacer)(n)-biotin(m)-polyacrylamide. These biotinylated derivatives can attach to lectins on the mucosal surface to facilitate absorption of the biologically active agent(s), e.g., a polymer-encapsulated NMDP.

### Bioadhesive Delivery Vehicles and Methods

In some embodiments, the combinatorial formulations and/or coordinate administration methods herein incorporate an effective amount of a nontoxic bioadhesive as an adjunct compound or carrier to enhance mucosal delivery of one or more biologically active agent(s). Bioadhesive agents in this context exhibit general or specific adhesion to one or more components or surfaces of the targeted mucosa. This enhancement of epithelial permeation often permits effective transmucosal delivery of large macromolecules, for example to the basal portion of the nasal epithelium or into the adjacent extracellular compartments or a blood plasma or CNS tissue or fluid. In some embodiments, the bioadhesive agents herein disclosed are useful in the combinatorial formulations and coordinate administration methods of the instant disclosure, which optionally incorporate an effective amount and form of a bioadhesive agent to prolong persistence or otherwise increase mucosal absorption of one or more NMDP, and other biologically active agents. The bioadhesive agents can be coordinately administered as adjunct compounds or as additives within the combinatorial formulations of this disclosure. In certain embodiments, the bioadhesive agent acts as a ‘pharmaceutical glue,’ whereas in other embodiments adjunct delivery or combinatorial formulation of the bioadhesive agent serves to intensify contact of the biologically active agent with the nasal mucosa, in some embodiments by promoting specific receptor-ligand interactions with epithelial cell “receptors,” and in others by increasing epithelial permeability to significantly increase the drug concentration gradient measured at a target site (e.g., liver, blood plasma, or CNS tissue or fluid). Yet additional bioadhesive agents within this disclosure act as enzyme (e.g., protease) inhibitors to enhance the stability of mucosally administered biotherapeutic agents delivered coordinately or in a combinatorial formulation with the bioadhesive agent.

The potential of various bioadhesive polymers as a mucosal, e.g., nasal, delivery platform within the methods and compositions of this disclosure can be readily assessed by determining their ability to retain and release NMDP, as well as by their capacity to interact with the mucosal surfaces following incorporation of the active agent therein. In addition, well known methods are applied to determine the biocompatibility of selected polymers with the tissue at the site of mucosal administration. When the target mucosa is covered by mucus (i.e., in the absence of mucolytic or mucus-clearing treatment), it can serve as a connecting link to the underlying mucosal epithelium. Therefore, the term “bioadhesive” as used herein also covers mucoadhesive compounds useful for enhancing mucosal delivery of biologically active agents within this disclosure. However, adhesive contact to mucosal tissue mediated through adhesion to a mucus gel layer can be limited by incomplete or transient attachment between the mucus layer and the underlying tissue, particularly at nasal surfaces where rapid mucus clearance occurs. In this regard, mucin glycoproteins are continuously secreted and, immediately after their release from cells or glands, form a viscoelastic gel. The luminal surface of the adherent gel layer, however, is continuously eroded by mechanical, enzymatic and/or ciliary action. Where such activities are more prominent or where longer adhesion times are desired, the coordinate administration methods and combinatorial formulation methods of this disclosure can further incorporate mucolytic and/or ciliostatic methods or agents as disclosed herein above.

Typically, mucoadhesive polymers for use within the present disclosure are natural or synthetic macromolecules which adhere to wet mucosal tissue surfaces by complex, but non-specific, mechanisms. In addition to these mucoadhesive polymers, this disclosure also describes methods and compositions incorporating bioadhesives that adhere directly to a cell surface, rather than to mucus, by means of specific, including receptor-mediated, interactions. One example of bioadhesives that function in this specific manner is the group of compounds known as lectins.

In certain aspects of this disclosure, bioadhesive materials for enhancing intranasal delivery of biologically active agents comprise a matrix of a hydrophilic, e.g., water soluble or swellable, polymer or a mixture of polymers that can adhere to a wet mucous surface. These adhesives can be formulated as ointments, hydrogels (see above) thin films, and other application forms. Often, these adhesives have the biologically active agent mixed therewith to effectuate slow release or local delivery of the active agent. Some are formulated with additional ingredients to facilitate penetration of the active agent through the nasal mucosa, e.g., into the auric channels of the individual.

Various polymers, both natural and synthetic ones, show significant binding to mucus and/or mucosal epithelial surfaces under physiological conditions. The strength of this interaction can readily be measured by mechanical peel or shear tests. When applied to a humid mucosal surface, many dry materials will spontaneously adhere, at least slightly. After such an initial contact, some hydrophilic materials start to attract water by adsorption, swelling or capillary forces, and if this water is absorbed from the underlying substrate or from the polymer-tissue interface, the adhesion can be sufficient to achieve the goal of enhancing mucosal absorption of biologically active agents. Such ‘adhesion by hydration’ can be quite strong, but formulations adapted to employ this mechanism must account for swelling which continues as the dosage transforms into a hydrated mucilage, e.g., some cellulose-derivatives, which are generally non-adhesive when applied in pre-hydrated state. In some embodiments, bioadhesive drug delivery systems for mucosal administration are effective within this disclosure when such materials are applied in the form of a dry polymeric powder, microsphere, or film-type delivery form.

Acrylic-based hydrogels are well suited for bioadhesion due to their flexibility and nonabrasive characteristics in the partially swollen state, which reduce damage-causing attrition to the tissues in contact. In some embodiments, the methods and compositions of this disclosure optionally include the use of carriers, e.g., polymeric delivery vehicles that function in part to shield the biologically active agent from proteolytic breakdown, while at the same time providing for enhanced penetration of the peptide or protein into or through the nasal mucosa., e.g., mucoadhesive poly(acrylic acid) derivative polycarbophil.

Other mucoadhesive polymers within this disclosure, for example chitosan, reportedly enhance the permeability of certain mucosal epithelia even when they are applied as an aqueous solution or gel. Another mucoadhesive polymer reported to directly affect epithelial permeability is hyaluronic acid and ester derivatives thereof. Chitosan is the N-deacetylated product of chitin, a naturally occurring polymer that has been used extensively to prepare microspheres for oral and intra-nasal formulations. Within one aspect of this disclosure, o-methylisourea is used to convert a chitosan amine to its guanidinium moiety. The guanidinium compound is prepared, for example, by the reaction between equi-normal solutions of chitosan and o-methylisourea at pH above 8.0.

In some embodiments, the bioadhesive is a lectin. Lectins are (glyco) proteins of non-immune origin which bind to polysaccharides or glycoconjugates. Several plant lectins have been investigated as possible pharmaceutical absorption-promoting agents. One plant lectin, Phaseolus vulgaris hemagglutinin (PHA), exhibits high oral bioavailability of more than 10% after feeding to rats. Tomato (Lycopersicon esculeutum) lectin (TL) appears safe for various modes of administration.

### Microemulsion Delivery Method

In some embodiments, the NMDP intranasal delivery formulation can comprise a microemulsion systems stabilized by nonionic surfactants such as Cremophor RH 40 or Labrasol, and can contain a variety of oils, including isopropyl myristate, Labrafil M 1944CS or Maisine 35-1. The formulation can comprise 8% Labrafil M 1944CS, 30% Cremophor RH 40/ethanol (3:1) and water, with a solubility of NMDP up to 6.4 mg/ml, droplet size of 30.3+/−5.3 nm, and no ciliotoxicity. After a single intranasal administration of such a preparation at a dose of 2 mg/kg, a plasma concentration can peak (e.g., Cmax) at 1 h and comprise an absolute bioavailability of about 32%.

### Liposomes and Micellar Delivery Vehicles

The coordinate administration methods and combinatorial formulations of the instant disclosure optionally incorporate effective lipid or fatty acid based carriers, processing agents, or delivery vehicles, to provide improved formulations for mucosal delivery of NMDP, and other biologically active agents. For example, a variety of formulations and methods are provided for mucosal delivery which comprise one or more of these active agents, additionally a peptide or protein, admixed or encapsulated by, or coordinately administered with, a liposome, mixed micellar carrier, or emulsion, to enhance chemical and physical stability and increase the half-life of NMDP and other active agents herein upon mucosal delivery.

In some embodiments, delivery systems for biologically active agents comprise small lipid vesicles known as liposomes. These can be made from natural, biodegradable, non-toxic, and non-immunogenic lipid molecules, and can efficiently entrap or bind drug molecules, into, or onto, their membranes.

In other embodiments, nimodpine and other active agents described herein can combine the use of polymers and liposomes to ally the advantageous properties of both vehicles such as encapsulation inside the natural polymer fibrin and controllable release through the use of covalent crosslinking and the addition of antifibrinolytic agents to the fibrin polymer. In other embodiments, liposomes can comprise cationic lipids, long and medium chain fatty acids, as well as surfactant mixed micelles with fatty acids, long chain fatty acids, fusogenic lipids, unsaturated fatty acids and monoglycerides such as oleic acid, linoleic acid, linoleic acid, monoolein, etc., medium chain fatty acids (C6 to C12) and monoglycerides, sodium salts of medium chain fatty acids (C6 to 12) and carriers to enhance mucosal delivery of NMDP, and other biologically active agents disclosed herein. The fatty acids can be employed in soluble forms of sodium salts or by the addition of non-toxic surfactants, e.g., polyoxyethylated hydrogenated castor oil, sodium taurocholate, etc. Fatty acid and mixed micellar preparations contemplated within this disclosure include, but are not limited to, Na caprylate (C8), Na caprate (C10), Na laurate (C12) or Na oleate (C18), optionally combined with bile salts, such as glycocholate and taurocholate.

### Pegylation

In alternate embodiments, NMDPs salts together with other biologically active peptides and proteins, are conjugated to polyalkylene oxide polymers, particularly polyethylene glycols (PEG). U.S. Pat. No. 4,179,337. PEG polymers contemplated within this disclosure include SC-PEG with molecular masses of 2000, 5000, 10000, 12000, and 20 000; U-PEG-10000; NHS-PEG-3400-biotin; T-PEG-5000; T-PEG-12000; and TPC-PEG-5000.

The section headings used herein are for organizational purposes only and are not to be construed as limiting the subject matter described.

### I. EXAMPLES

The following examples are included for illustrative purposes only and are not intended to limit the scope of the disclosure.

### Example 1: NMDP Intranasal Formulation

In this Example, NMDP is dissolved in a solution containing a carrier, a citrate buffer, benzalkonium chloride, and water using ultrasound to obtain a solution containing about 50 mg/mL of NMDP. 50 μL of the composition is then administered into each nasal cavity of a subject using an intranasal drug delivery device. Blood samples are then harvested at 0, 2, 5, 10, 15, 30 and 60 minutes, and the NMDP concentration determined by high performance liquid chromatography (HPLC). The pharmacokinetics of NMDP delivery via intranasal administration can then be compared with the pharmacokinetics of NMDP delivery by intravenous administration. The pharmacokinetics of the intranasally administered NMDP are comparable to that of intravenously administered NMDP.

### Example 2: NMDP Intranasal Formulation with Surfactant

In this Example, NMDP is dissolved in a solution containing one or more natural or synthetic carriers, in an amount from about 30% to about 95% (w/w); a non-aqueous solvent from about 10% to about 70% (w/w); and a surfactant using ultrasound to obtain a solution containing about 50 mg/mL of NMDP. 50 μL of the composition is then administered into each nasal cavity of a subject using an intranasal drug delivery device. Blood samples are then harvested at 0, 2, 5, 10, 15, 30 and 60 minutes, and the NMDP concentration determined by high performance liquid chromatography (HPLC). The pharmacokinetics of NMDP delivery via intranasal administration can then be compared with the pharmacokinetics of NMDP delivery by intravenous administration. The pharmacokinetics of the intranasally administered NMDP are comparable to that of intravenously administered NMDP.

### Example 3: NMDP Intranasal Formulation without Surfactant

In this Example, NMDP is dissolved in a solution comprising about 60% to about 80% (w/w) mPEG350, about 10% to about 30% (w/w) PEG400, about 5% to about 15% (w/w) water, and without a non-ionic surfactant. 50 μL of the composition comprising about 100 mg/ml of NMDP is then administered into each nasal cavity of a subject using an intranasal drug delivery device. Blood samples are then harvested at 0, 2, 5, 10, 15, 30 and 60 minutes, and the NMDP concentration determined by high performance liquid chromatography (HPLC). The pharmacokinetics of NMDP delivery via intranasal administration can then be compared with the pharmacokinetics of NMDP delivery by intravenous administration. The pharmacokinetics of the intranasally administered NMDP are comparable to that of intravenously administered NMDP.

### Example 4: NMDP Intranasal Formulation without Surfactant and with Ethanol

In this Example, NMDP is dissolved in a solution comprising about 60% to about 80% (w/w) mPEG350, about 10% to about 30% (w/w) PEG400, about 5% to about 15% (w/w) water, about 5% to about 15% ethanol, and without a non-ionic surfactant. 50 μL of the composition comprising about 100 mg/mL of NMDP is then administered into each nasal cavity of a subject using an intranasal drug delivery device. Blood samples are then harvested at 0, 2, 5, 10, 15, 30 and 60 minutes, and the NMDP concentration determined by high performance liquid chromatography (HPLC). The pharmacokinetics of NMDP delivery via intranasal administration can then be compared with the pharmacokinetics of NMDP delivery by intravenous administration. The pharmacokinetics of the intranasally administered NMDP are comparable to that of intravenously administered NMDP.

### Example 5: Manufacture and Intranasal Formulation of NMDP

Nasal compositions of NMDP can be manufactured for administration as a medicament for administration to a patient for one of the indications described herein. Briefly, NMDP, buffer, benzalkonium chloride and optionally other ingredients (such as sodium chloride or other osmolarity-regulating agent, sorbitol or other sweetener, flavoring agent, etc.) can be made up to some volume less than the target final volume of the solution. The ingredients can then be mixed until all the ingredients are dissolved. The pH then can be adjusted, if necessary, by addition of a suitable acid or base, such as HCl, NaOH, or the complementary acid or base of the buffer. Once the desired pH has been obtained, the solution can then be brought up to full volume with water. The resulting solution can then be packaged in a suitable container for shipping and distribution. In some embodiments, the suitable container includes a nasal pump. In other embodiments, the suitable container can be a vial, such as an amber glass vial, which can be a glass ampule, a glass bottle topped with an inert rubber septum and crimp cap top, or other suitable pharmaceutical vial.

### Example 6: Treating Tinnitus with NMDP

A subject suffering from tinnitus is treated with about 1 to 10 mg/kg of intranasally delivered NMDP. The subject is treated with the intranasal delivery formulation of Example 15, F1 or F2. The time to Cmax is about 20 min. The subject notices a decrease in a ringing of the ear or other symptom of tinnitus following administration of the intranasal formulation. The subject readministers the formulation as needed to treat the symptoms of tinnitus, approximately every 2-4 hours. A lower amount of NMDP is required to be administered to the subject using an intranasal delivery formulation then is required by oral administration.

### Example 7: Intranasal Administration of NDMP

Six volunteer subjects suffering from tinnitus are treated with about 1 to 10 mg/kg of intranasally delivered NMDP. Each subject was treated with 50 μL of a nasal spray preparation per nare, prepared as described in Example 5, every two to four hours. The 100 μL dose delivered about 20 mg NMDP. at 0, 2, 5, 10, 15, 30 and 60 minutes, and the NMDP concentration determined by high performance liquid chromatography (HPLC). Time to Cmax is about 1 hr. The pharmacokinetics of NMDP delivery via intranasal administration can then be compared with the pharmacokinetics of NMDP delivery by intravenous administration. The pharmacokinetics of the intranasally administered NMDP are comparable to that of intravenously administered NMDP.

### Example 8: Intranasal Vesicular Composition

In this Example, about 2500 mg of NMDP is dissolved in a solution containing a phospholipid, one or more C2-C4 alcohols and water to obtain a solution containing about 200 mg/mL of NMDP. The composition comprises vesicles present therein, whose size ranging between 50 nm to few microns, and more specifically, up to 5 μm, exhibiting good properties for enhanced nasal absorption. The vesicles are then visualized by transmission electron microscopy (TEM) and scanning electron microscopy. TEM analysis is then carried out using an electron microscope with an accelerating voltage of 100 kV.

50 μL of the composition is then administered into each nasal cavity of a subject using an intranasal drug delivery device. Blood samples are then harvested at 0, 2, 5, 10, 15, 30 and 60 minutes, and the NMDP concentration determined by high performance liquid chromatography (HPLC). The pharmacokinetics of NMDP delivery via intranasal administration can then be compared with the pharmacokinetics of NMDP delivery by intravenous administration. The pharmacokinetics of the intranasally administered NMDP are comparable to that of intravenously administered NMDP.

### Example 9: Dry Powder NMDP Formulation

About 2500 mg of NMDP is dissolved in a mixture of acetone (12 g) and ethanol (12 g) under stirring at 300 rpm. An appropriate size magnetic bar is placed in the receiver and lactose monohydrate (2.3 g) is added. The stirring rate is set at 150 rpm. The clear and homogeneous solution of the active agent (sumatriptan succinate) is spray-dried with inlet air temperature of 60° C. and outlet temperature of 55° C., thereby obtaining the dry powder of the active agent, which is further blended with lactose monohydrate in-situ in the receiver. Stirring is maintained during the entire process. The actual weight of NMDP as an active agent in the obtained sumatriptan/lactose composition is about 15. % w/w. The composition is then mixed with an additional amount of lactose in order to reach the required 10% active agent (NMDP) concentration.

### Example 10: Mucoadhesive Formulation

A 20-g batch of mucoadhesive, gel formulation containing 2.0% of NMDP is prepared by suspending 20.0 mg of Carbopol 934P and 1.80 g of Poloxamer 188 (BASF Corp.) in 5.00 g of TRIS HCl buffer (0.1 M) and the components are mixed under agitation overnight at 4° C. to ensure complete dissolution. Additional components, including hydroxypropyl methylcellulose (100.0 mg), methylparaben (10 mg) and additional TRIS HCl buffer (0.1 M) (2.87 g) are added and further stirring allowed until complete dissolution is observed. Tacrolimus (100 g), in addition to NMDP, is added and mixed while maintaining activity. The mixture is maintained below room temperature until use.

### Example 11: Microemulsion Formulation

NMDP is dissolved in a microemulsion systems stabilized by nonionic surfactants to a concentration of about 50 mg/mL Cremophor RH 40 and Labrasol, and oils, including isopropyl myristate, Labrafil M 1944CS, and Maisine 35-1. The formulation comprises 8% Labrafil M 1944CS, 30% Cremophor RH 40/ethanol (3:1) and water, with a maximum solubility of NMDP up to 6.4 mg/ml, droplet size of 30.3+/−5.3 nm, and no ciliotoxicity.

50 μL of the composition is then administered into each nasal cavity of a subject using an intranasal drug delivery device. Blood samples are then harvested at 0, 2, 5, 10, 15, 30 and 60 minutes, and the NMDP concentration determined by high performance liquid chromatography (HPLC). The pharmacokinetics of NMDP delivery via intranasal administration can then be compared with the pharmacokinetics of NMDP delivery by intravenous administration. After a single intranasal administration of this preparation at a dose of 2 mg/kg, the plasma concentration peaked at 1 h and the absolute bioavailability was about 32%.

### Example 12: Treatment of Induced Tinnitus in Mice

In this example, mice suffering from noise induced tinnitus are treated with an intranasal delivery formulation of NMDP, e.g., the formulation of Example 1.

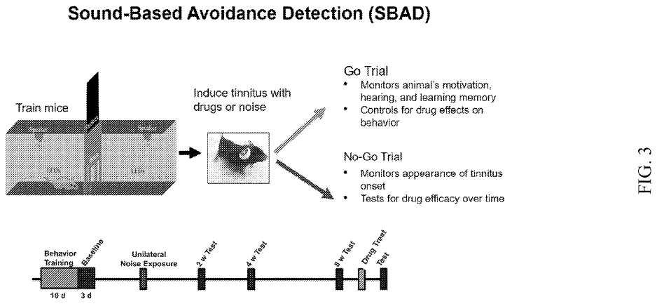

A sound-based avoidance detection (SBAD) method, as illustrated in FIG. 3, was used for detecting tinnitus and testing the animals' response to different pharmacological doses of NMDP. Using a shuttle box divided into two compartments, mice were trained to cross from side to side with sound cues in the “Go” trials and remain still in the absence of sound in the “No-Go” trials for 15 days. The animals had a 5 minute acclimation period in the shuttle box before testing began with 100 randomly assigned trials per day, lasting approximately 30-40 minutes. The sound cues were randomly played as white noise or narrow-band noise at 5, 10, 16, 20, and 32 kHz with sound intensities randomly at 75, 80, or 85 dB. To reinforce the training sessions, the mice were shocked if they did not cross from one compartment to the other during the Go trials, and as well in the No-Go trials if they moved compartments in the absence of sound. Additionally, a hurdle was added on training 11 to reinforce the training. After the 15th training day, mice were tested for 3 days to obtain a baseline score to be compared after noise exposure, as well as retraining for 3 days.

Following the successful completion of training so that mice reached a high percentage of success in the trials, the mice were exposed to a noise-induced trauma for tinnitus. To ensure that the mice still have their hearing, one ear was protected with an ear plug, while the other ear was exposed to the noise. The mice were placed in a sound booth where they were exposed to 120 dB broadband noise at 4-25 kHz for 2-4 hours. Following the noise exposure, the mice were housed for one month, and then tested to see if they have developed tinnitus. In these testing periods, the mice were tested for three days in the shuttle boxes, where they underwent the Go and No-Go trials, however in the No-Go trials the mice were not shocked, as it is expected that mice with tinnitus would cross compartments in the absence of a sound cue. The mice were continued to be shocked in the Go trials in order to ensure that they have retained their hearing and can hear the sound cues. In examining the results of the tests following noise exposure, tinnitus positive mice had a higher average of errors in the No-Go trials in comparison to their training. To insure there is a significant difference in the No-Go trial scores, chi-square tests were completed comparing the post-noise exposure tests to the baseline tests. However, if tinnitus is not present in the mice, tests were done after waiting one more month, and another if needed.

Mice received dosages of NMDP initially via intraperitoneal injection (i.p.). The Go and No-Go scores for the tests were compared using chi-square tests in Social Science Statistics. The chi-square tests were used first to determine whether mice were tinnitus positive by comparing the No-Go baseline scores pre-noise exposure or saline-injected to the No-Go post-noise exposure scores, with a significant difference (p-value<0.05) in at least 2 of the tests indicating a tinnitus-positive mouse. Following this conclusion, the mice were tested accordingly to determine if their No-Go scores could be improved by different drug doses and combinations. In the Go scores, NMDP did not have an effect on the mice's ability to hear the sounds presented. In the No-Go trials, NMDP did not affect the mice's scores, indicating that the concentrations at 10, 30, and 50 mg/kg (i.P.) did not cause any behavioral changes and should be appropriate doses for testing.

The No-Go scores at 3 months post-noise exposure were compared to baselines to assess for tinnitus. Mice had significant decreases on all 3 days of testing. This indicates these mice had tinnitus. One or more mice is a control animal. In looking at the Go scores for the tinnitus animals when tested with the specified drugs, mice had significant increase in their No-Go correct scores after NMDP treatment, indicating NMDP is effective in treating this mouse's tinnitus.

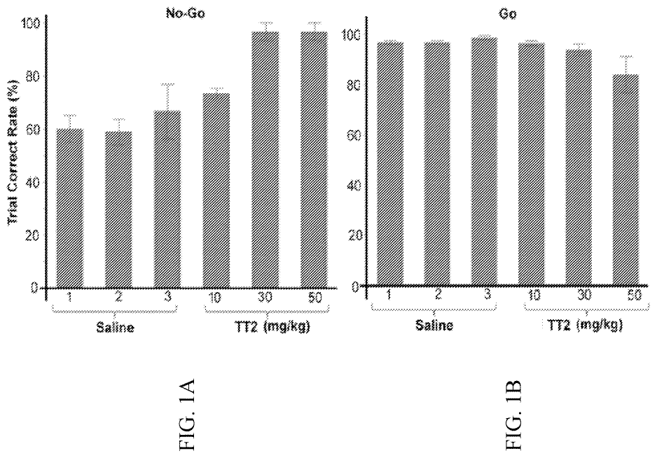

FIGS. 1A and 1B illustrate the results of the No-Go and Go trials for the mice treated (i.p.) with 10, 30, and 50 mg/kg of NMDP. In FIG. 1A, it is illustrated that the mice showed only a modest improvement at a dosage of 10 mg/kg which was within the margin of error of at least one of the saline candidates; the mice showed a significant improvement approaching nearly 100% correct at dosages of 30 mg/kg and 50 mg/kg, indicating that the mice's tinnitus was in fact being reduced by the 30 mg/kg and 50 mg/kg dosages of the NMDP. Further, in FIG. 1B it is illustrated that the saline treated mice as well as the 10 mg/kg and 30 mg/kg NMDP treated mice had nearly the same 100% correct rate in the Go trial, indicating that the mice were in fact completing the trials as they were trained to do, and that the data produced from the experiment was reliable. The 50 mg/kg treated mice saw a lower correct rate in the Go trial, possibly indicating that they were beginning to experience some level of drug induced side effects from the 50 mg/kg dosage of NMDP.

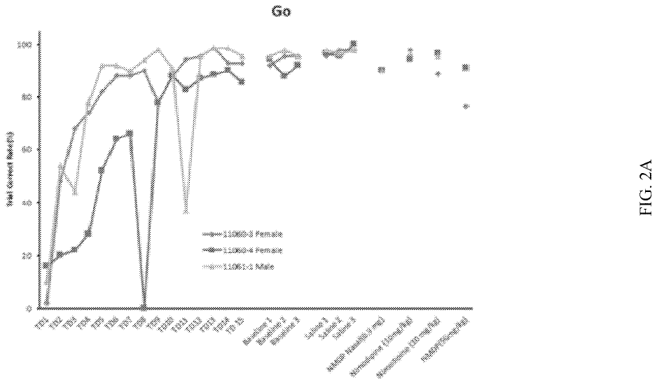

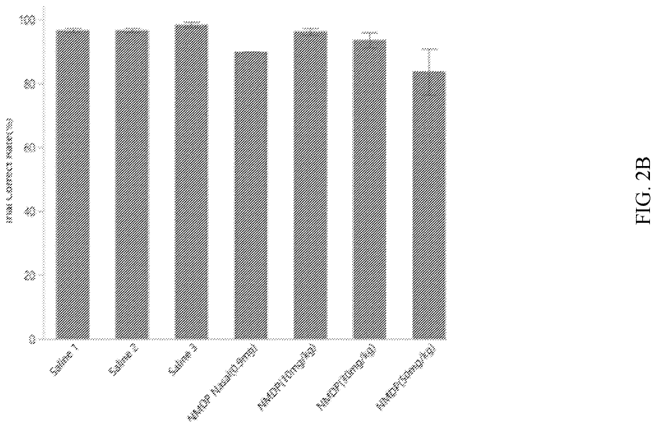

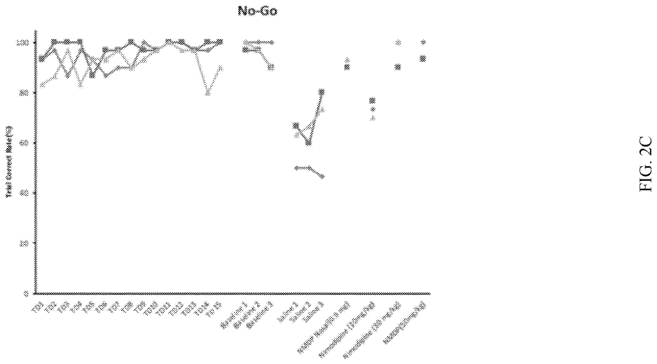

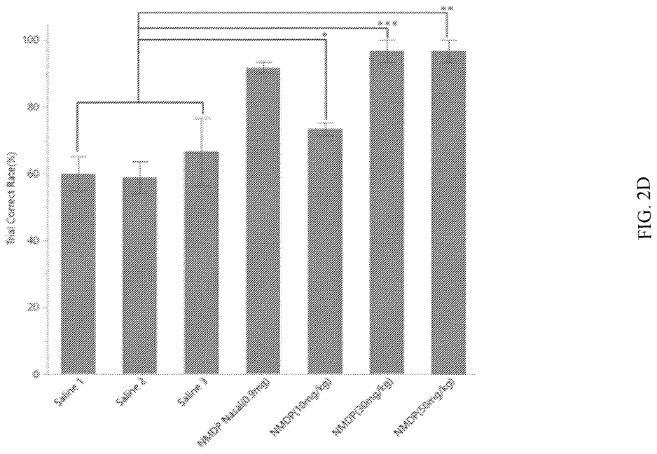

FIGS. 2A and 2C are plots illustrating the results of the performances of individual mouse on the Go and No-Go trials at each specific trial, at baseline, when treated with saline, when treated with NMDP at dosages of 10 mg/kg, 30 mg/kg, and 50 mg/kg, and when treated with NMDP with the intranasal formulation of Example 15 F1 at a dosage of 0.9 mg/mouse (or 30 mg/kg with averaged mouse weight at 30 g at this stage, about 5 to 6 months old). FIGS. 2B and 2D are bar graphs illustrating the percentage correct rates of the mice's performances on the Go and No-Go trials. As can be observed in FIG. 2B for the Go trial, the saline treated mice as well as the 10 mg/kg and 30 mg/kg NMDP treated mice had nearly the same 100% correct rate in the Go trial, indicating that the mice were in fact completing the trials as they were trained to do, and that the data produced from the experiment was reliable. The 50 mg/kg and 0.9 mg/mouse intranasally treated mice saw a lower correct rate in the Go trial, possibly indicating that they were beginning to experience some level of drug induced side effects. As can be observed in FIG. 2C for the No-Go trial, the mice showed only a modest improvement at a dosage of 10 mg/kg which was within the margin of error of at least one of the saline candidates; the mice showed a significant improvement approaching nearly 100% correct at dosages of 30 mg/kg, 50 mg/kg, and the intranasally delivered dosage of 0.9 mg/mouse, indicating that the mice's tinnitus was in fact being treated by the 30 mg/kg, 50 mg/kg, and 0.9 mg/mouse.

Further, the close correlation between the reduced correct rate in FIG. 2C between the 50 mg/kg (i.p.), and 0.9 mg/mouse intranasally treated mice (possibly indicated a drug induced side effect); and the close correlation between the significantly improved correct rate in FIG. 2D between the 30 mg/kg, 50 mg/kg, and the 0.9 mg/mouse (indicating effective treatment of the tinnitus), indicates that the 0.9 mg/mouse intranasally delivered dosage of NMDP had a similar effect upon the mice as the 30 mg/kg, 50 mg/kg dosages administered by intraperitoneal injection. A 0.9 mg intranasally delivered dosage of NMDP only corresponds to an approximate 30 mg/kg dosage of NMDP, providing strong evidence of the utility of the intranasal delivery formulation, as it was as effective as the 30 mg/kg and 50 mg/kg dosages administered by intraperitoneal injection, indicating that a significantly reduced dosage of NMDP can be utilized when administered via an intranasal formulation.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

What is claimed is:

1. A method for treating a hearing disorder or a symptom of a hearing disorder in a subject in need thereof comprising intranasally administering a therapeutically effective amount of NMDP or an L-type calcium channel blocker to the subject.

2. The method of claim 1, wherein the hearing disorder is tinnitus or Meniere's disease.

3. A method for treating a hearing disorder or a symptom of a hearing disorder in a subject in need thereof comprising:
a. administering an L-type calcium channel blocker to the subject;
b. assessing if the subject was responsive to the L-type calcium channel blocker; and
c. administering a therapeutically effective amount of NMDP or a salt thereof to the subject if the subject was responsive to the L-type calcium channel blocker.

4. The method of claim 3, wherein the hearing disorder is tinnitus or Meniere's disease.

5. A method of selecting a subject in need thereof for treatment of tinnitus or Meniere's disease or a symptom of tinnitus or Meniere's disease comprising:
a. administering an L-type calcium channel blocker to the subject;
b. selecting the patient for treatment of tinnitus or Meniere's disease or a symptom of tinnitus or Meniere's disease if the subject was responsive to the L-type calcium channel blocker; and
c. administering a therapeutically effective amount of NMDP or a salt thereof to the subject if the subject was responsive to the L-type calcium channel blocker.

6. The method of any one of claim 3 or 5, wherein the L-type calcium channel blocker comprises carbamazepine.

7. The method of any one of claim 3 or 5, further comprising intranasally administering the therapeutically effective amount of NMDP or a salt thereof to the patient for treatment of tinnitus or Meniere's disease or a symptom of tinnitus or Meniere's disease.

8. The method of claim 7, wherein the therapeutically effective amount of NMDP is administered in a composition comprising:
a. the therapeutically effective amount of NMDP;
b. a carrier;
c. a citrate buffer;
d. benzalkonium chloride; and
e. ethylenediamine tetraacetic acid

9. The method of claim 7, wherein the therapeutically effective amount of NMDP is administered in a composition comprising:
a. the therapeutically effective amount of NMDP;
b. one or more natural or synthetic carriers, or any combinations thereof, in an amount from about 10% to about 95% (w/w); and
c. a non-aqueous solvent from about 10% to about 95% (w/w); and
d. a surfactant.

10. The method of claim 7, wherein the therapeutically effective amount of NMDP administered in a composition comprising at least one of:
a. polyethyleneglycol;
b. methoxy-polyethylene glycol; and
c. water.

11. The method of any of claims 1-5, wherein the therapeutically effective amount of NMDP is from about 0.1 mg to about 5 mg per kg of the subject.

12. The method of any one of claims 1-11, further comprising administering the pharmaceutical composition in a volume from about 10 μl to about 300 μl per dose.

13. The method of any one of claims 1-12, further comprising bringing at least a portion of the therapeutically effective amount of the pharmaceutical composition into contact with the mucosal membrane of at least one nasal cavity.

14. The method of any one of claims 1-13, further comprising spraying a first quantity of the pharmaceutical composition into the first nasal cavity, spraying a second quantity of the pharmaceutical composition into a second nasal cavity, and optionally after a pre-selected time delay, spraying a third quantity of the pharmaceutical composition into the first nasal cavity.

15. The method of claim 14, further comprising, optionally after a pre-selected time delay, administering at least a fourth quantity of the pharmaceutical composition to the second nostril.

16. The method of any one of claims 1-15, wherein said method of treatment achieves bioavailability that is from about 80%-125% of that achieved with the same pharmaceutical composition administered intravenously.

17. The method of any one of claims 1-16, further comprising administering the pharmaceutical composition at any time before or after onset of symptoms of tinnitus or Meniere's disease.

18. The method of any one of claims 1-17, further comprising administering the pharmaceutical composition at least once a day, twice a day, three times a day, four times a day, five times a day, six times a day, seven times a day, eight times a day, nine times a day, or ten times a day.

19. The method of any of claims 1-5, wherein the therapeutically effective amount of NMDP is administered intranasally every 2 hours.

20. The method of any of claims 1-5, wherein the therapeutically effective amount of NMDP is administered intranasally every 4 hours.

21. The method of any of claims 1-5, further comprising administering the pharmaceutical composition weekly.

22. The method of any one of claims 1-12, further comprising administering the pharmaceutical composition via a low-dose therapy.

23. The method of any one of claims 1-22, further comprising administering the pharmaceutical composition by titration to vestibular symptoms.

24. The method of any one of claims 1-23, further comprising administering the pharmaceutical composition for at least 1, 2, 3, 4, 5, 6, 14, 21, 28, 60, 120, or 400 days.

25. The method of any one of claims 1-24, further comprising administering the pharmaceutical composition in a unit dosage form.

26. The method of any one of claims 1-25, further comprising administering the pharmaceutical composition in order to not cause a change in cochlear potential.

27. The method of any one of claims 1-26, further comprising increasing the subject's dose of the pharmaceutical composition until a symptom of inner ear disturbance is observed.

28. The method of claim 27, wherein the symptom of inner ear disturbance comprises spontaneous nystagmus observed with Frenzel's glasses, disequilibrium, motion intolerance, or reduction/loss in hearing.

29. The method of any one of claims 1-27, further comprising administering the pharmaceutical composition via drug delivery device.

30. The method of any one of claims 1-29, wherein the effect of the pharmaceutical composition is measured by an assessment selected from the group consisting of a) measurement of a change in ABR and/or DPOAE amplitude and threshold, b) a change in auditory speech recognition as measured by a words-in-noise test, c) a change in auditory speech recognition as measured by a digits-in-noise test, d) a change in low-frequency hearing threshold e) a change in incidence of adverse events after administration of the pharmaceutical composition, f) a change in tinnitus or Meniere's disease severity g) a change in tinnitus or Meniere's disease loudness h) a change in vertigo severity i) a change in aural fullness j) a change in dizziness, and k) a change in hair cell function as observed when measured by a change in ABR threshold, after administration of the pharmaceutical composition.

31. The method of claim 30, wherein the measurement of the ABR threshold is performed at the frequency range of 250 Hz-20 KHz.

32. A method of achieving a therapeutically effective area under the curve (AUC) extrapolated to infinity from dosing time (AUC0-infinity) of NMDP in a subject in need thereof, comprising intranasally administering an intranasal pharmaceutical composition to the subject, wherein the intranasal pharmaceutical composition comprises:
(i) from about 0.2 to about 250 mg/kg NMDP or a pharmaceutically acceptable salt thereof;
(ii) a buffer; and
(iii) a surfactant,
wherein the subject exhibits an AUC0-infinity of NMDP which is between 270 h*ng/ml and 340 h*ng/mL following administration of the intranasal pharmaceutical composition to the subject.

33. A pharmaceutical composition comprising a therapeutically effective amount of NMDP, wherein composition is formulated for intranasal administration, the composition comprising:
a. a therapeutically effective amount of NMDP;
b. a buffer;
c. a penetration enhancer; and
d. a surfactant.

34. The pharmaceutical composition of claim 33, wherein the composition further comprises one or more of the ingredients selected from vitamin E, vitamin E TPGS, ethanol, benzyl alcohol, and dodecyl maltoside.

35. The pharmaceutical composition of any one of claim 33 or 34, wherein the concentration of NMDP in the pharmaceutical composition administered is 0.5, 1, 1.5, 2, 2.5, 3, 3.5, 4, 4.5, 5, 5.5, 6, 6.5, 7, 7.5, 8, 8.5, 9, 9.5, 10, 12.5, 15, 17.5, 20, 22.5, 25, 27.5, 30, 32.5, 35, 37.5, 40, 42.5, 45, 47.5, 50, 55, 60, 65, 70, 75, 80, 85, 90, 95, 100 mg/ml.

36. The pharmaceutical composition of any one of claims 33-35, wherein the volume of the pharmaceutical composition administered is 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1.0 ml.

37. The pharmaceutical composition of any one of claims 33-35, wherein the pharmaceutical composition is an extended-release formulation.

38. The pharmaceutical composition of any one of claims 33-35, wherein the pharmaceutical composition is a sustained release formulation.

39. The pharmaceutical composition of any one of claims 33-35, wherein the pharmaceutical composition is a controlled release formulation.

40. The pharmaceutical composition of any one of claims 33-35, wherein the pharmaceutical composition is released either continuously, variably, or in a pulsatile manner, or combinations thereof.

41. The pharmaceutical composition of any one of claims 33-35, wherein the pharmaceutical composition is in a unit dosage form.

42. The pharmaceutical composition of claim 41, wherein the unit dosage form is in a dry powder, semi-solid, a mucoadhesive formulation, intranasal vesicular unit or solution dosage form.

43. The pharmaceutical composition of claim 42, wherein the composition is aqueous.

44. The pharmaceutical composition of claim 43, wherein the composition is in the form of gel or film.

45. The pharmaceutical composition of any one of claims 33-44, wherein the composition comprises micronized particles.

46. The pharmaceutical composition of claim 41, wherein the unit dosage form has a unit weight of from about 10 mg to about 10 g.

47. The method of claim 42, wherein the solution dosage form has a unit dosage volume of less than about 600 μL.

48. The pharmaceutical composition of any one of claims 33-47, comprising NMDP at a concentration of from about 0.1% to about 20% w/w of the formulation.

49. The pharmaceutical composition of any one of claims 33-48, wherein the pharmaceutical composition prolongs the residence time of the composition in an auris structure.

50. The pharmaceutical composition of claim 49, wherein the formulation prolongs the residence time of the composition in the auris structures for a period of at least 1 day, at least 2 days, at least 3 days, at least 4 days, at least 5 days, at least 6 days, at least 8 days, or at least 14 days, at least 21 days, or at least 1 month, or at least 6 weeks after a single administration.

51. The pharmaceutical composition of claim 33, wherein the formulation increases the bioavailability of the composition in the auris structure.

52. The pharmaceutical composition of claim 51, wherein the formulation increases the steady state levels of the composition in the auris structure.

53. The pharmaceutical composition of claim 52, wherein the formulation increases the time to reach Cmax of the therapeutic concentration that is capable of reducing the symptom of the hearing disorder in the subject in need thereof.

54. The pharmaceutical composition of claim 53, wherein the formulation prolongs the time that the concentration of the composition will stay above the minimum therapeutic concentration (i.e., Cmin) necessary for reducing the symptom of the hearing disorder in the subject in need thereof.

55. The pharmaceutical composition of claim 54, wherein the concentration of the composition in the auris structures stays at or about concentrations greater than Cmin for a period of at least 3 days, 4 days, 5 days, 6 days, 7 days, 10 days, 14 days, 3 weeks or 1 month.

56. The pharmaceutical composition of any one of claims 33-55, further comprising a second pharmaceutical active agent.

57. A pharmaceutical composition comprising a therapeutically effective amount of NMDP, composition formulated for intranasal administration, the composition comprising:
a. a therapeutically effective amount of NMDP; and
b. a surfactant;
c. a solvent; and
d. a water-soluble cellulosic polymer.

58. The pharmaceutical composition of claim 57, further comprising water.

59. The pharmaceutical composition of any one of claims 57-58, further comprising ethanol.

60. The pharmaceutical composition of any one of claims 57-59, wherein the ethanol is at most 5% (w/w).

61. The pharmaceutical composition of any one of claims 57-60, wherein the surfactant is a polysorbate or a combination of polysorbates.

62. The pharmaceutical composition of any one of claims 57-61, wherein the surfactant is polysorbate 20 (TWEEN-20),

63. The pharmaceutical composition of any one of claims 57-61, wherein the surfactant is polysorbate 80 (TWEEN-80).

64. The pharmaceutical composition of any one of claims 57-61, wherein the surfactant is at least 0.00001% and at most 2% (w/w).

65. The pharmaceutical composition of any one of claims 57-64, wherein the surfactant is at least 0.05% (w/w) and at most 2% (w/w).

66. The pharmaceutical composition of any one of claims 57-65, wherein the water-soluble cellulosic polymer is HPMC, HEC, CMC, carboxymethylcellulose sodium, or a combination thereof.

67. The pharmaceutical composition of any one of claims 57-66, wherein the water-soluble cellulosic polymer is at least 0.00001% and at most 2% (w/w).

68. The pharmaceutical composition of any one of claims 57-67, wherein the water-soluble cellulosic polymer is at least 0.05% (w/w) and at most 2% (w/w).

69. The pharmaceutical composition of any one of claims 57-68, wherein the solvent is PEG, an alkoxy derivative of polyethylene glycol, mPEG, mPEG350, PEG400, or a combination thereof.

70. The pharmaceutical composition of any one of claims 57-69, wherein the solvent is at least 0.00001% and at most 94% (w/w).

71. The pharmaceutical composition of any one of claims 57-69, wherein the solvent is at least 66% (w/w) and at most 94% (w/w).

72. The pharmaceutical composition of any one of claims 57-71, wherein the water is at least 0.00001% and at most 50% (w/w).

73. The pharmaceutical composition of any one of claims 57-72, wherein the water is at least 0.00001% and at most 30% (w/w).

74. The pharmaceutical composition of any one of claim 57-73, wherein the water is at least 5% (w/w) and at most 50% (w/w).

75. The pharmaceutical composition of any one of claim 57-74, wherein the water is at least 5% (w/w) and at most 30% (w/w).

76. A pharmaceutical composition comprising a therapeutically effective amount of NMDP, wherein the composition is formulated for intranasal administration, the composition comprising:
a. a therapeutically effective amount of NMDP;
b. a surfactant;
c. a water-soluble cellulosic polymer;
d. a solvent; and
e. water.

77. The pharmaceutical composition of claim 76, wherein the pharmaceutical composition further comprises three solvents.

78. The pharmaceutical composition of any one of claims 76-77, wherein the three solvents comprise at least three of PEG, an alkoxy derivative of polyethylene glycol, mPEG, mPEG350, PEG400, or a combination thereof.

79. The pharmaceutical composition of any one of claims 76-78, wherein the surfactant is a polysorbate or a combination of polysorbates.

80. The pharmaceutical composition of any one of claims 76-79, wherein the surfactant is polysorbate 20 (Tween-20).

81. The pharmaceutical composition of any one of claims 76-79, wherein the surfactant is polysorbate 80 (Tween-80).

82. The pharmaceutical composition of claim 81, wherein the polysorbate 80 (Tween-80) is at least 0.00001% and at most 2% (w/w).

83. The pharmaceutical composition of claim 81-82, wherein the polysorbate 80 (Tween-80) is at least 0.05% (w/w) and at most 2% (w/w).

84. The pharmaceutical composition of any one of claims 73-83, wherein the water-soluble cellulosic polymer is HPMC, HEC, CMC, carboxymethylcellulose sodium, or a combination thereof.

85. The pharmaceutical composition of any one of claims 73-84, wherein the water-soluble cellulosic polymer only includes HPMC.

86. The pharmaceutical composition of claim 76-85, wherein the HPMC is at least 0.00001% and at most 2% (w/w).

87. The pharmaceutical composition of claim 76-86, wherein the HPMC is at least 0.05% (w/w) and at most 2% (w/w).

88. The pharmaceutical composition of any one of claims 73-87, wherein the solvent is PEG, an alkoxy derivative of polyethylene glycol, mPEG, mPEG350, PEG400, or a combination thereof.

89. The pharmaceutical composition of any one of claims 73-88, wherein the PEG, an alkoxy derivative of polyethylene glycol, mPEG, mPEG350, PEG400, or a combination thereof is at least 0.00001% and at most 94% (w/w).

90. The pharmaceutical composition of any one of claims 73-89, wherein the PEG, an alkoxy derivative of polyethylene glycol, mPEG, mPEG350, PEG400, or a combination thereof is at least 66% (w/w) and at most 94% (w/w).

91. The pharmaceutical composition of any one of claims 73-90, wherein the water is at least 0.00001% and at most 50% (w/w).

92. The pharmaceutical composition of any one of claim 73-91, wherein the water is at least 5% (w/w) and at most 50% (w/w).

93. The pharmaceutical composition of any one of claims 73-92, wherein the water is at least 0.00001% and at most 30% (w/w).

94. The pharmaceutical composition of any one of claim 73-93, wherein the water is at least 5% (w/w) and at most 30% (w/w).

95. A process for preparing an NMDP liquid composition, the process comprising the steps of:
1) dissolving Tween 80 (<2% w/w) separately in mPEG350 and PEG400;
2) mixing NMDP with the solutions from Step 1) to saturation solubility, or a desired effective concentration;
3) preparing an aqueous solution of HPMC or HPMC in buffer (<2% w/w) with a pH value in the range of about 6.4 to about 7.4; and
3) adding an aqueous HPMC solution or HPMC buffer from Step 3) to the respective solutions from Step 2) with agitation and allow the mixtures to stand to observe solution or suspension stability.

96. The process of claim 95, wherein the Step 2) solution is NMDP in a PEG/Tween 80 solution or NMDP in a mPEG/Tween 80 solution.

97. The pharmaceutical composition of any one of claims 57-96, wherein the concentration of NMDP is at least 68 mg/mL.

98. The pharmaceutical composition of any one of claims 57-97, wherein the concentration of NMDP is at least 0.1 mg/mL up to 1 mg/mL.

99. The pharmaceutical composition of any one of claims 57-98, wherein the concentration of NMDP is 68 mg/mL.

100. The pharmaceutical composition of any one of claims 57-99, wherein the concentration of NMDP is 50 mg/mL.

101. The pharmaceutical composition of any one of claims 57-100, wherein the surfactant, water-soluble cellulosic polymer, solvent, water and ethanol comprise a liquid vehicle.

102. The pharmaceutical composition of any one of claims 57-101, wherein the liquid vehicle is less than 1000 μL.

103. The pharmaceutical composition of any one of claims 57-102, wherein the liquid vehicle is less than 300 μL.

104. The pharmaceutical composition of any one of claims 57-103, wherein the liquid vehicle is less than 150 μL.

105. The pharmaceutical composition of any one of claims 57-104, wherein the NMDP is dissolved, suspended, or both dissolved and suspended in the liquid vehicle.

106. A pharmaceutical composition comprising a therapeutically effective amount of NMDP, wherein the composition is formulated for intranasal administration, the composition comprising:
a. a therapeutically effective amount of NMDP; and
b. a liquid vehicle, wherein the liquid vehicle further comprises
i. a surfactant;
ii. a water-soluble cellulosic polymer;
iii. a solvent; and
iv. water.

107. The pharmaceutical composition of claim 76 or 96, wherein:
a. the liquid vehicle further comprises:
i. the surfactant;
ii. the water-soluble cellulosic polymer;
iii, the solvent; and
iv. the water.

108. A pharmaceutical composition comprising a therapeutically effective amount of NMDP, wherein composition is formulated for intranasal administration, the composition comprising:
a. a therapeutically effective amount of NMDP;
b. a solvent; and
c. water.

109. The pharmaceutical composition of claim 108, wherein the solvent is PEG, an alkoxy derivative of polyethylene glycol, mPEG, mPEG350, PEG400, or a combination thereof.

110. The pharmaceutical composition of any one of claims 108-109, wherein the solvent is at least 0.00001% and at most 94% (w/w).

111. The pharmaceutical composition of any one of claims 108-110, wherein the solvent is at least 66% (w/w) and at most 94% (w/w).

112. The pharmaceutical composition of any one of claims 108-111, wherein the water is at least 0.00001% and at most 20% (w/w).

113. The pharmaceutical composition of claim 108, wherein the composition further comprises a first solvent and a second solvent.

114. The pharmaceutical composition of claim 113, wherein the first solvent comprises mPEG and the second solvent comprises PEG.

115. The pharmaceutical composition of claim 114, wherein the mPEG comprises mPEG350.

116. The pharmaceutical composition of claim 114, wherein the PEG comprises PEG400.

117. The pharmaceutical composition of claim 108, wherein the therapeutically effective amount of NMDP comprises at least 10 mg/mL, at least 15 mg/mL, at least 20 mg/mL, at least 25 mg/mL, at least 30 mg/mL, at least 35 mg/mL, at least 40 mg/mL, at least 45 mg/mL, at least 50 mg/mL, at least 55 mg/mL, at least 60 mg/mL, at least 65 mg/mL, at least 70 mg/mL, at least 80 mg/mL, at least 90 mg/mL, at least 95 mg/mL, at least 100 mg/mL, at least 110 mg/mL, or greater than 110 mg/mL.

118. The pharmaceutical composition of claim 114, wherein the mPEG comprises about 20% to about 90%, about 30% to about 90%, about 40% to about 90%, about 50% to about 90%, about 60% to about 90%, about 70% to about 90% or about 80% to about 90% (w/w) of the composition, the PEG comprises about 10% to about 50%, about 20% to about 50%, about 30% to about 50%, or about 40% to about 50% (w/w) of the composition, and the water comprises about about 5% to about 20% about 10% to about 20%, or about 15% to about 20% (w/w) of the composition.

119. The pharmaceutical composition of claim 108, wherein the composition further comprises ethanol.

120. The pharmaceutical composition of claim 119, wherein the ethanol comprises about 1% to about 10% about 2% to about 10%, about 3% to about 10%, about 4% to about 10%, about 5% to about 10%, about 6% to about 10%, about 7% to about 10%, about 8% to about 10%, or about 9% to about 10% of the composition.
